(19) United States
(12) Patent Application Publication
(10) Pub. No.: US 2024/0265999 A1
(43) Pub. Date: Aug. 8, 2024

(54) METHODS AND SYSTEMS FOR METAGENOMICS ANALYSIS

(71) Applicants: Heng XIE, Salt Lake City, UT (US); Steven FLYGARE, Salt Lake City, UT (US); Qing LI, Salt Lake City, UT (US); Wan XIE, Salt Lake City, UT (US); Robert SCHLABERG, Salt Lake City, UT (US); IDbyDNA Inc., Salt Lake City, UT (US)

(72) Inventors: Heng Xie, Salt Lake City, UT (US); Steven Flygare, Salt Lake City, UT (US); Qing Li, Salt Lake City, UT (US); Wan Xie, Salt Lake City, UT (US); Robert Schlaberg, Salt Lake City, UT (US); Yuying Mei, Salt Lake City, UT (US); Guochun Liao, Salt Lake City, UT (US); Hajime Matsuzaki, Sal Lake City, UT (US)

(21) Appl. No.: 18/003,663

(22) PCT Filed: Jan. 24, 2022
(86) PCT No.: PCT/US2022/013562

§ 371 Date: Dec. 28, 2022

Related U.S. Application Data
(60) Provisional application No. 63/140,436, filed on Jan. 22, 2021.

(51) Int. Cl. G16B 30/10; G16B 20/20

(52) U.S. Cl. CPC G16B 30/10; G16B 20/20

(57) ABSTRACT

Systems and methods for identifying conditions in a sample obtain a set of sample sequence reads from the sample. For each respective read, or respective sample contig derived from a respective subset of the set, a corresponding sequence comparison between the respective read or contig and each reference sequence in a set of reference sequences is performed. There is calculated, from these sequence comparisons, a respective probability that the respective read or contig corresponds to a particular reference sequence in the set of reference sequences thereby computing a plurality of probabilities. The presence or an absence of each of the conditions in the sample is identified based at least in part on these probabilities. One condition is identification of a species present in the sample, and the percentage of the genome of this species identified in the reads is provided.

### CROSS REFERENCE TO RELATED APPLICATIONS

This application claims benefit of U.S. Provisional Patent Application No. 63/140,436, entitled “Methods and Systems for Metagonomic Analysis,” filed Jan. 22, 2021, which is hereby incorporated by reference.

### BACKGROUND

Metagenomics, the genomic analysis of a population of microorganisms, makes possible the profiling of microbial communities in the environment and the human body at unprecedented depth and breadth. Its rapidly expanding use is revolutionizing our understanding of microbial diversity in natural and man-made environments and is linking microbial community profiles with health and disease. To date, most studies have relied on PCR amplification of microbial marker genes (e.g. bacterial 16S rRNA), for which large, curated databases have been established. More recently, higher throughput and lower cost sequencing technologies have enabled a shift towards enrichment-independent metagenomics. These approaches reduce bias, improve detection of less abundant taxa, and enable discovery of novel pathogens.

While pathogen-specific nucleic acid amplification tests may be highly sensitive and specific, they may require a priori knowledge of likely pathogens. The result is increasingly large, yet inherently limited diagnostic panels to enable diagnosis of the most common pathogens. In contrast, enrichment-independent or highly multiplexed enrichment-based high-throughput sequencing allows for unbiased, hypothesis-free detection and molecular typing of a theoretically unlimited number of common and unusual pathogens. Wide availability of next-generation sequencing instruments, lower reagent costs, and streamlined sample preparation protocols are enabling an increasing number of investigators to perform high-throughput DNA and RNA-seq for metagenomics studies. However, analysis of sequencing data is still forbiddingly difficult and time consuming, requiring bioinformatics skills, computational resources, and microbiological expertise that is not available to many laboratories, especially diagnostic ones.

In view of the foregoing, more computationally efficient, accurate, and easy-to-use tools for comprehensive diagnostic and metagenomics analyses are needed.

### SUMMARY

The methods and systems described herein address the need for more computationally efficient, accurate, and easy-to-use tools for comprehensive diagnostic and metagenomics analyses, and provide other advantages as well.

One aspect of the present disclosure provides a computer system comprising one or more processors, memory, and one or more programs. The one or more programs are stored in the memory and are configured to be executed by the one or more processors. The one or more programs are for identifying a presence or an absence of one or more conditions in a first sample from a sample source.

The one or more programs comprise a classification module. The classification module includes instructions for A(i) obtaining, in electronic form, a set of sample sequence reads for a plurality of polynucleotides from the first sample. The set of sequence reads comprises at least 50,000 sequence reads. The classification module also includes instructions for A(ii) performing, for each respective sequence read in the set of sample sequence reads or a respective sample contig derived from a respective subset of the set of sample sequence reads, a corresponding sequence comparison between at least a portion of the respective sample sequence read or respective sample contig and each reference sequence in a first set of reference sequences, where the first set of reference sequences comprises 1000 or more reference sequences, thereby performing a first plurality of sequence comparisons. The classification module also includes instructions for A(iii) performing, dependent or independent of when the performing A(ii) is completed, for each respective sequence read in the set of sample sequence reads or a respective sample contig derived from a respective subset of the set of sample sequence reads, a corresponding sequence comparison between at least a portion of the respective sample sequence read or respective sample contig and each reference sequence in a second set of reference sequences, wherein the second set of reference sequences comprises 1000 or more reference sequences, thereby performing a second plurality of sequence comparisons. The classification module also includes instructions for A(iv) calculating, from the first and second plurality of sequence comparisons, a respective probability that the respective sample sequence read or the respective sample contig corresponds to a particular reference sequence in the first or second set of reference sequences thereby computing a first plurality of probabilities. The classification module also includes instructions for A(v) identifying a presence or an absence of each of the one or more conditions in the sample based at least in part on the first plurality of probabilities.

The one or more programs also comprise a quality control module. The quality control module instructions for B(i) obtaining, in electronic form, a control set of control sequence reads or control contigs for a plurality of control polynucleotides from a second sample. The control set of control sequence reads or control contigs comprises at least 10,000 control sequence reads or control contigs. The quality control module also includes instructions for B(ii) performing, for each respective control sequence read or control contig in the control set of control sequence reads or control contigs, a corresponding sequence comparison between at least a portion of the respective control sequence read or control contig and each reference sequence in the first or second set of reference sequences, thereby performing a second plurality of sequence comparisons. The quality control module also includes instructions for B(iii) calculating, from the second plurality of sequence comparisons, a respective probability that the respective control sequence read or control contig corresponds to a particular reference sequence in the set of reference sequences thereby computing a second plurality of probabilities. The quality control module also includes instructions for B(iv) confirming the identification of the presence or an absence of each of the one or more conditions in the sample when the second plurality of probabilities indicates that the control set of control sequences or control contigs (i) exhibit a predetermined condition that the second sample is known to have or (ii) does not exhibit a predetermined condition that the second sample is known to not have.

In some embodiments, the performing (A)(ii) comprises forming a respective plurality of k-mers that represent the respective sample sequence read or sample contig and comparing each k-mer to a corresponding plurality of weighted k-mers representing a reference sequence, in polynucleotide form, in the first set of reference sequences, where a respective weighted k-mer (Kᵢ) in the corresponding plurality of weighted k-mers for a reference sequence (refᵢ) in the first set of reference sequences has a higher weight (KWrefᵢ) when it is a less prevalent k-mer across the reference sequence, in polynucleotide form, and a respective weighted k-mer (Kᵢ) in the corresponding plurality of weighted k-mers for a reference sequence (refᵢ) in the set of reference sequences has a lower weight KWrefᵢ when it is a more prevalent k-mer across the reference sequence, in polynucleotide form. In some such embodiments, a k-mer weight of a respective weighted k-mer in the corresponding plurality of weighted k-mers for a reference sequence relates to a count of a particular k-mer within a particular reference sequence, a count of the particular k-mer among a group of sequences comprising the reference sequence, and a count of the particular k-mer among all reference sequences in the set of reference sequences.

In some embodiments a respective weighted k-mer (Kᵢ) in the corresponding plurality of weighted k-mers for a reference sequence (refᵢ) in the first set of reference sequences has a higher weight (KWrefᵢ) when it is a less prevalent k-mer across the first set of reference sequences, in polynucleotide form, and a respective weighted k-mer (Kᵢ) in the corresponding plurality of weighted k-mers for a reference sequence (refᵢ) in the first set of reference sequences has a lower weight KWrefᵢ when it is a more prevalent k-mer across the reference sequence, in polynucleotide form.

In some embodiments, the first set of reference sequences are protein sequences and the one or more programs further comprise instructions for translating the first set of reference sequence to polynucleotide form.

In some embodiments, KWrefᵢ is calculated as:

$$K W r e {f}_{i} = \frac{{C}_{r e f} ( {K}_{i} ) / {C}_{db} ( {K}_{i} )}{{C}_{db} ( {K}_{i} ) / Total kmer count}$$

where C_{ref}(Kᵢ) is a count of a number of occurrences of the respective weighted k-mer (Kᵢ) in the respective reference sequence (refᵢ), Cab (Kᵢ) is a count of a number of occurrences of the respective k-mer (Kᵢ) in the first set of reference sequences, and Total kmer count is a number of k-mers of length k-nucleotides in the first set of reference sequences.

In some embodiments, each k-mer in the respective plurality of k-mers has k contiguous nucleotides of the respective sequence read, wherein k is an integer between 2 and 50, between 2 and 45, between 2 and 40, between 2 and 35, between 5 and 30, between 10 and 25, or between 12 and 20.

In some embodiments, the calculating A(iv) calculates the respective probability that the respective sample sequence read or sample contig corresponds to a particular reference sequence using the sequence comparison of each k-mer in the respective sequence read.

In some embodiments, the sample source is a test subject and the set of sample sequence reads or sample contigs for the plurality of polynucleotides and the sample are deidentified from an identity of the subject. In some such embodiments, the test subject and the set of sample sequence reads or sample contigs are deidentified from the identity of the subject using a bar code that uniquely represents the subject. In some embodiments, the first set of reference sequences all originate from one genus and the one or more programs further comprises a lookup table that equates the deidentified sample to the identity of the test subject.

In some embodiments, each reference sequence in the first set of reference sequences is from a first genus and, each reference sequence in the second set of reference sequences is from a second genus.

In some embodiments, each reference sequence in the first set of reference sequences is bacterial, and each reference sequence in the second set of reference sequences is human.

In some embodiments, each reference sequence in the first set of reference sequences is viral, and each reference sequence in the second set of reference sequences is human.

In some embodiments, each reference sequence in the first set of reference sequences is microbial, and each reference sequence in the second set of reference sequences is mammalian.

In some embodiments, the first set of reference sequences comprises reference sequences from 10 or more species. In some embodiments, the first set of reference sequences comprises reference sequences from between 2 and 100 species, between 3 and 500 species, or between 2 and 1000 species.

In some embodiments, a condition in the one or more conditions is presence of nucleic acids or proteins in the first sample from a particular taxa. In some such embodiments, the sample source is a test subject and the particular taxa is a domain, a sub-domain, a kingdom, a sub-kingdom, a phylum, a sub-phylum, a class, a sub-class, an order, a sub-order, a family, a subfamily, a genus, a subgenus, or a species.

In some embodiments, the sample source is a test subject and a condition in the one or more conditions is presence of an expression profile, a particular gene, a particular antimicrobial resistance gene, a particular antiviral resistance gene, a particular antivirulent resistance gene, a particular antiparasitic resistant gene, or a particular antiprotozoal resistance gene in the first sample.

In some embodiments, the sample source is a test subject and a condition in the one or more conditions is a likely disease progression for the test subject, a drug resistance exhibited by the test subject, a pathogenicity exhibited by the test subject, increased predisposition to a disease exhibited by the test subject, or decreased predisposition to a disease exhibited by the test subject.

In some embodiments, a condition in the one or more conditions is a taxa and the taxa comprises a first bacterial strain identified as present in the sample source and a second bacterial strain identified as absent from the sample source.

In some embodiments, the first set of reference sequences consist of between 100 and 1×10⁶ groups of sequences, and each respective group of sequences is associated with a different bacterial or viral contaminant and each condition in the one or more conditions corresponds to a different group in the between 100 and 1×10⁶ groups of sequences. In some such embodiments, the second set of reference sequences consist of human sequences. In some such embodiments, a first group in the between 100 and 1×10⁶ groups of sequences represents a first bacterial or viral strain and is identified as present in the first sample and a second group in the between 100 and 1×10⁶ groups of sequences represents a second bacterial or viral strain and is identified as absent in the first sample.

In some embodiments, the first set of reference sequences comprises sequences from a plurality of taxa, and a reference sequence in the first set of reference sequences is associated with a reference k-mer weight indicative of a likelihood that a reference k-mer within the reference polynucleotide sequence originates from a taxon.

In some embodiments, the first set of reference sequences includes reference sequences for 10, 50, 100, 1000, 10000, 100000, 1000000, or more conditions. In some such embodiments, each condition represented in the first set of reference sequences is a corresponding set of one or more genetic variants in a particular species. In some such embodiments, each corresponding set of one or more genetic variants includes a single nucleotide polymorphism (SNP), a deletion/insertion polymorphism (DIP), a copy number variant (CNV), a short tandem repeat (STR), a restriction fragment length polymorphism (RFLP), a simple sequence repeat (SSR), a variable number of tandem repeat (VNTR), a randomly amplified polymorphic DNA (RAPD), an amplified fragment length polymorphisms (AFLP), a mter-retrotransposon amplified polymorphism (IRAP), a long and short interspersed element (LINE/SINE), a long tandem repeat (LTR), a mobile element, a retrotransposon microsatellite amplified polymorphism, a retrotransposon-based insertion polymorphism, a sequence specific amplified polymorphism, or an epigenetic modification. In some such embodiments, each corresponding set of one or more genetic variants includes an epigenetic modification (e.g., a methylation status at an allele that is associated with a biological state). In some such embodiments, the biological state is cancer.

In some embodiments, the corresponding sequence comparison of A(ii) and A(iii) is performed under exact matching stringency.

In some embodiments, the one or more programs further comprises instructions for determining an absolute or relative abundance of a composition, associated with a condition in the one or more conditions, in the first sample. In some such embodiments, the absolute or relative abundance of a composition is an amount of a particular polynucleotide in the first sample. In some such embodiments, the particular polynucleotide has a polymorphism. In some embodiments the absolute or relative abundance of the composition is an amount of a particular protein in the first sample.

In some embodiments, the one or more conditions is a single condition.

In some embodiments, the one or more conditions is between two and 150 different conditions.

In some embodiments, the one or more conditions is a single condition, the sample source is a first subject, the first set of reference sequences includes reference sequences for a plurality of subjects, and the confirming the identification of the presence or an absence of each of the one or more conditions in the sample confirms the first subject as being a particular subject represented in the plurality of subjects. In some such embodiments, the plurality of subjects comprises 10², 10³, 10⁴, 10⁵, 10⁶, 10⁷, 10⁸, or 10⁹ subjects.

In some embodiments, the A(ii) is performed in parallel for 2, 3, 4, 5, 6, 7, 8, 9, or 10 or more of the 10 or more, 100 or more, 200 or more, 1000 or more, or 10,000 or more sample sequence reads in the set of sample sequence reads or 2, 3, 4, 5, 6, 7, 8, 9, or 10 or more of the 10 or more, 100 or more, 200 or more, 1000 or more, or 10,000 or more sample contigs derived from the set of sample sequence reads.

In some embodiments, the first set of reference sequences comprises reference sequences of one or more of bacteria, archaea, chromalveolata, viruses, fungi, plants, fish, amphibians, reptiles, birds, mammals, and humans.

In some embodiments, the first set of reference sequences consists of sequences from a reference individual or a reference sample source. In some such embodiments, the one or more programs further include instructions for identifying the polynucleotides from the sample source as being derived from the reference individual or the reference sample source using the first or second plurality of probabilities.

In some embodiments, the first the set of reference sequences comprises k-mers having one or more mutations with respect to one or more known polynucleotide sequences, such that a plurality of variants of the one or more known polynucleotide sequences are represented in the first set of reference sequences.

In some embodiments, the first set of reference sequences comprises a plurality of marker gene sequences for taxonomic classification of bacterial sequences. In some such embodiments, the plurality of marker gene sequences comprises 16S rRNA sequences. In some such embodiments, the first set of reference sequences comprises sequences of human transcripts, and wherein a condition in the one or more conditions is an indication as to whether a sequence read in the set of sequence reads is derived from a human subject.

In some embodiments, the one or more conditions is a first condition, and the first set of reference sequences consists of sequences associated with the first condition. In some such embodiments the computer system further comprise instructions for identifying the sample source as having the first condition.

In some embodiments, the sample source is a first subject, the (B)(iv) confirming determines that the subject has a first condition in the one or more conditions, and the first condition is an infection, and the one or more programs further include instructions for monitoring treatment in the first subject by identifying the presence or absence of a biosignature in samples from the infected first subject at multiple times after beginning treatment. In some such embodiments, the one or more programs further include instructions for providing notice to change treatment of the infected subject based on results of the monitoring.

In some embodiments, the first set of reference sequences comprises polynucleotide sequences reverse-translated from amino acid sequences. In some such embodiments, the reverse-translating uses a non-degenerate code comprising a single codon for each amino acid. In some such embodiments, a sequence read is translated to an amino acid sequence and then reverse-translated using the non-degenerate code prior to comparison with the reverse-translated reference sequences.

In some embodiments, a user uploads the set of sequence reads to the computer system, and the A(ii) performing is executed concurrently with the upload.

In some embodiments, the (A)(ii) performing performs the sequence comparison at a rate of at least 1×10⁶, 2×10⁶, 3×10⁶, 4×10⁶, 5×10⁶, 10×10⁶, 20×10⁶, 30×10⁶, 40×10⁶, or 50×10⁶ sample sequence reads per minute for the sample sequence reads in the set of sample sequence reads.

In some embodiments, the one or more programs further comprise instructions for removing from the set of sample sequence, prior to the A(ii) performing and A(iii) performing, each respective sample sequence read that fails to satisfy a quality metric threshold. In some such embodiments, the quality metric threshold is a read quality for the respective sample sequence read or a length of the sample sequence read. In some embodiments, the quality metric threshold is a sample sequence read length and the respective sample sequence read is removed from the set of sample sequence reads when it is short than a cut off distance. In some such embodiments, the cut off distance is set by a user and is between 50-1000 nucleotides, between 60-500 nucleotides, between 70-400 nucleotides, between 80-300 nucleotides, between 90-200 nucleotides, or between 100-150 nucleotides.

In some embodiments, the first set of reference sequences comprises reference sequences for at least 50, 100, 250, 500, 1000, 5000, 10000, 50000, 100000, 250000, 500000, or 1000000 different genes.

In some embodiments, the sample sequence reads giving rise to the confirmation of the identification of the presence or an absence of a condition in the one or more conditions represent less than 0.01 percent, less than 0.001 percent, less than 0.0001 percent, less than 0.00001 percent, less than 0.000001 percent or less than 0000001 percent of the sample sequence reads in the set of sample sequence reads.

In some embodiments, the classification module performs the sequence comparisons against the first and second set of reference sequences concurrently.

In some embodiments, the classification module performs the sequence comparisons against the first and second set of reference sequences sequentially.

In some embodiments, the performing A(iii) is performed independent of when the performing A(ii) is completed.

In some embodiments, the performing A(iii) is performed concurrent to the performing A(ii).

In some embodiments, the performing A(iii) is performed dependent of when the performing A(ii) is completed.

In some embodiments, the performing A(iii) is performed after the performing A(ii) is completed.

In some embodiments, the classification module further comprises instructions for comparing each sequence read in the set of sample sequence reads to each reference sequence of between 3 and 1000 additional sets of reference sequences, between 10 and 500 additional sets of reference sequences, or between 20 and 400 additional sets of reference sequences.

In some embodiments, the first set of reference sequences are nucleotide sequences, the second set of reference sequences are protein sequence, each sequence comparison performed by the A(ii) sequence comparison is a nucleotide sequence to nucleotide sequence comparison, and each sequence comparison performed by the A(iii) sequence comparison is an amino acid sequence to amino acid sequence comparison in which the respective sample sequence read or sample contig has been translated to an amino acid sequence. In some such embodiments, the A(iii) sequence comparison is performed for each of six different reference frames of the respective sample sequence read or respective sample contig.

In some embodiments, the set of sample sequence reads comprise RNA and DNA sequences.

In some embodiments, the set of sample sequence reads consists of RNA sequences.

In some embodiments, the set of sample sequence reads consists of DNA sequences.

In some embodiments, a condition in the one or more conditions is an identification of a first species present in the first sample, and the one or more programs further comprises instructions for showing a percentage of a genome of the first species identified by the (A)(ii) in the set of sample sequence reads.

In some embodiments, each respective condition in the one or more conditions is an identification of a corresponding species in a plurality of species identified as present in the first sample, and the one or more programs further comprises instructions for showing a respective percentage of a corresponding genome identified by the (A)(ii) in the set of sample sequence reads for each species in the plurality of species. In some such embodiments, the plurality of species is between two and one hundred species. In some such embodiments, the plurality of species include viral and bacterial species.

In some embodiments, the first sample and the second sample are the same sample.

In some embodiments, the first sample and the second sample are different samples.

In some embodiments the one or more conditions are specified by a first diagnostic test profile. In some such embodiments, the one or more programs further comprise instructions for selecting the first diagnostic test profile from a plurality of diagnostic test profiles. In some such embodiments, the plurality of diagnostic test profiles comprises 10 or more, 50 or more, or 100 or more diagnostic test profiles.

In some embodiments, the one or more conditions are specified by a user selected disease or disease category from among a plurality of diseases or disease categories.

Another aspect of the present disclosure provides a method for identifying a presence or an absence of one or more conditions in a first sample from a sample source. The method can use a computer system comprising one or more processing cores and a memory that execute a classification module and a quality control module. In some embodiments the classification module and the quality control module are in the same program. In some embodiments the classification module and the quality control module are independent programs. In some embodiments, the classification module A(i) obtains, in electronic form, a set of sample sequence reads for a plurality of polynucleotides from the first sample, wherein the set of sequence reads comprises at least 50,000 sequence reads. In some embodiments, the classification module also A(ii) performs, for each respective sequence read in the set of sample sequence reads or a respective sample contig derived from a respective subset of the set of sample sequence reads, a corresponding sequence comparison between at least a portion of the respective sample sequence read or respective sample contig and each reference sequence in a first set of reference sequences, wherein the first set of reference sequences comprises 1000 or more reference sequences, thereby performing a first plurality of sequence comparisons. In some embodiments, the classification module also A(iii) performs, dependent or independent of when the performing A(ii) is completed, for each respective sequence read in the set of sample sequence reads or a respective sample contig derived from a respective subset of the set of sample sequence reads, a corresponding sequence comparison between at least a portion of the respective sample sequence read or respective sample contig and each reference sequence in a second set of reference sequences, where the second set of reference sequences comprises 1000 or more reference sequences, thereby performing a second plurality of sequence comparisons. In some embodiments, the classification module also A(iv) calculates, from the first and second plurality of sequence comparisons, a respective probability that the respective sample sequence read or the respective sample contig corresponds to a particular reference sequence in the first or second set of reference sequences thereby computing a first plurality of probabilities. In some embodiments, the classification module also A(v) identifies a presence or an absence of each of the one or more conditions in the sample based at least in part on the first plurality of probabilities.

In some embodiments, the quality control module B(i) obtains, in electronic form, a control set of control sequence reads or control contigs for a plurality of control polynucleotides from a second sample, where the control set of control sequence reads or control contigs comprises at least 10,000 control sequence reads or control contigs. In some embodiments, the quality control module also B(ii) performs, for each respective control sequence read or control contig in the control set of control sequence reads or control contigs, a corresponding sequence comparison between at least a portion of the respective control sequence read or control contig and each reference sequence in the first or second set of reference sequences, thereby performing a second plurality of sequence comparisons. In some embodiments, the quality control module also B(iii) calculates, from the second plurality of sequence comparisons, a respective probability that the respective control sequence read or control contig corresponds to a particular reference sequence in the set of reference sequences thereby computing a second plurality of probabilities. In some embodiments, the quality control module also B(iv) confirms the identification of the presence or an absence of each of the one or more conditions in the sample when the second plurality of probabilities indicates that the control set of control sequences or control contigs (i) exhibit a predetermined condition that the second sample is known to have or (ii) does not exhibit a predetermined condition that the second sample is known to not have.

Another aspect of the present disclosure provides a computer readable storage medium storing one or more programs. The one or more programs comprise instructions, which when executed by an electronic device with one or more processors and a memory cause the electronic device to perform a method for identifying a presence or an absence of one or more conditions in a first sample from a sample source. In some embodiments this method comprises executing a classification module. In some embodiments the classification module A(i) obtains, in electronic form, a set of sample sequence reads for a plurality of polynucleotides from the first sample, wherein the set of sequence reads comprises at least 50,000 sequence reads. In some embodiments the classification module also A(ii) performs, for each respective sequence read in the set of sample sequence reads or a respective sample contig derived from a respective subset of the set of sample sequence reads, a corresponding sequence comparison between at least a portion of the respective sample sequence read or respective sample contig and each reference sequence in a first set of reference sequences, where the first set of reference sequences comprises 1000 or more reference sequences, thereby performing a first plurality of sequence comparisons. In some embodiments the classification module also A(iii) performs, dependent or independent of when the performing A(ii) is completed, for each respective sequence read in the set of sample sequence reads or a respective sample contig derived from a respective subset of the set of sample sequence reads, a corresponding sequence comparison between at least a portion of the respective sample sequence read or respective sample contig and each reference sequence in a second set of reference sequences, wherein the second set of reference sequences comprises 1000 or more reference sequences, thereby performing a second plurality of sequence comparisons. In some embodiments the classification module also A(iv) calculates, from the first and second plurality of sequence comparisons, a respective probability that the respective sample sequence read or the respective sample contig corresponds to a particular reference sequence in the first or second set of reference sequences thereby computing a first plurality of probabilities. In some embodiments the classification module also A(v) identifies a presence or an absence of each of the one or more conditions in the sample based at least in part on the first plurality of probabilities.

In some embodiments this method also comprises executing a quality control module. In some embodiments, the quality control module B(i) obtains, in electronic form, a control set of control sequence reads or control contigs for a plurality of control polynucleotides from a second sample, where the control set of control sequence reads or control contigs comprises at least 10,000 control sequence reads or control contigs. In some embodiments, the quality control module also B(ii) performs, for each respective control sequence read or control contig in the control set of control sequence reads or control contigs, a corresponding sequence comparison between at least a portion of the respective control sequence read or control contig and each reference sequence in the first or second set of reference sequences, thereby performing a second plurality of sequence comparisons. In some embodiments, the quality control module also B(iii) calculates, from the second plurality of sequence comparisons, a respective probability that the respective control sequence read or control contig corresponds to a particular reference sequence in the set of reference sequences thereby computing a second plurality of probabilities. In some embodiments, the quality control module also B(iv) confirms the identification of the presence or an absence of each of the one or more conditions in the sample when the second plurality of probabilities indicates that the control set of control sequences or control contigs (i) exhibit a predetermined condition that the second sample is known to have or (ii) does not exhibit a predetermined condition that the second sample is known to not have.

Another aspect of the present disclosure provides a computer system comprising, one or more processors, memory; and one or more programs. The one or more programs are stored in the memory and configured to be executed by the one or more processors. The one or more programs are for identifying a presence or an absence of one or more conditions in a first sample from a sample source. The one or more programs comprise a classification module. The classification module includes instructions for A(i) obtaining, in electronic form, a set of sample sequence reads for a plurality of polynucleotides from the first sample, wherein the set of sequence reads comprises at least 50,000 sequence reads. The classification module also includes instructions for A(ii) performing, for each respective sequence read in the set of sample sequence reads or a respective sample contig derived from a respective subset of the set of sample sequence reads, a corresponding sequence comparison between at least a portion of the respective sample sequence read or respective sample contig and each reference sequence in a first set of reference sequences, where the first set of reference sequences comprises 1000 or more reference sequences, thereby performing a first plurality of sequence comparisons. The classification module also includes instructions for A(iii) performing, dependent or independent of when the performing A(ii) is completed, for each respective sequence read in the set of sample sequence reads or a respective sample contig derived from a respective subset of the set of sample sequence reads, a corresponding sequence comparison between at least a portion of the respective sample sequence read or respective sample contig and each reference sequence in a second set of reference sequences, where the second set of reference sequences comprises 1000 or more reference sequences, thereby performing a second plurality of sequence comparisons. The classification module also includes instructions for A(iv) calculating, from the first and second plurality of sequence comparisons, a respective probability that the respective sample sequence read or the respective sample contig corresponds to a particular reference sequence in the first or second set of reference sequences thereby computing a first plurality of probabilities. The classification module also includes instructions for A(v) identifying a presence or an absence of each of the one or more conditions in the sample based at least in part on the first plurality of probabilities. In some embodiments, a condition in the one or more conditions is an identification of a first species present in the first sample, and the one or more programs further comprises instructions for showing a percentage of a genome of the first species identified by the (A)(ii) in the set of sample sequence reads.

In some embodiments, each respective condition in the one or more conditions is an identification of a corresponding species in a plurality of species identified as present in the first sample, and the one or more programs further comprises instructions for showing a respective percentage of a corresponding genome identified by the (A)(ii) in the set of sample sequence reads for each species in the plurality of species. In some such embodiments, the plurality of species is between two and one hundred species. In some such embodiments, the plurality of species include viral and bacterial species.

In some embodiments, the one or more programs further comprise a quality control module. The quality control module includes instructions for B(i) obtaining, in electronic form, a control set of control sequence reads or control contigs for a plurality of control polynucleotides from a second sample, where the control set of control sequence reads or control contigs comprises at least 10,000 control sequence reads or control contigs. The quality control module further includes instructions for B(ii) performing, for each respective control sequence read or control contig in the control set of control sequence reads or control contigs, a corresponding sequence comparison between at least a portion of the respective control sequence read or control contig and each reference sequence in the first or second set of reference sequences, thereby performing a second plurality of sequence comparisons. The quality control module further includes instructions for B(iii) calculating, from the second plurality of sequence comparisons, a respective probability that the respective control sequence read or control contig corresponds to a particular reference sequence in the set of reference sequences thereby computing a second plurality of probabilities. The quality control module further includes instructions for B(iv) confirming the identification of the presence or an absence of each of the one or more conditions in the sample when the second plurality of probabilities indicates that the control set of control sequences or control contigs (i) exhibit a predetermined condition that the second sample is known to have or (ii) does not exhibit a predetermined condition that the second sample is known to not have.

In some embodiments, the one or more conditions are specified by a first diagnostic test profile. In some such embodiments, the one or more programs further comprise instructions for selecting the first diagnostic test profile from a plurality of diagnostic test profiles. In some such embodiments, the plurality of diagnostic test profiles comprises 10 or more, 50 or more, or 100 or more diagnostic test profiles.

In some embodiments, the one or more conditions are specified by a user selected disease or disease category from among a plurality of diseases or disease categories.

Another aspect of the present disclosure is a method for identifying a presence or an absence of one or more conditions in a first sample from a sample source. The method comprises using a computer system comprising one or more processing cores and a memory to (i) obtain, in electronic form, a set of sample sequence reads for a plurality of polynucleotides from the first sample, wherein the set of sequence reads comprises at least 50,000 sequence reads. The method also comprises using a computer system to (ii) perform, for each respective sequence read in the set of sample sequence reads or a respective sample contig derived from a respective subset of the set of sample sequence reads, a corresponding sequence comparison between at least a portion of the respective sample sequence read or respective sample contig and each reference sequence in a first set of reference sequences, where the first set of reference sequences comprises 1000 or more reference sequences, thereby performing a first plurality of sequence comparisons. The method further comprises using a computer system to (iii) perform, dependent or independent of when the performing A(ii) is completed, for each respective sequence read in the set of sample sequence reads or a respective sample contig derived from a respective subset of the set of sample sequence reads, a corresponding sequence comparison between at least a portion of the respective sample sequence read or respective sample contig and each reference sequence in a second set of reference sequences, where the second set of reference sequences comprises 1000 or more reference sequences, thereby performing a second plurality of sequence comparisons. The method further comprises using a computer system to (iv) calculate, from the first and second plurality of sequence comparisons, a respective probability that the respective sample sequence read or the respective sample contig corresponds to a particular reference sequence in the first or second set of reference sequences thereby computing a first plurality of probabilities. The method further comprises using a computer system to (v) identify a presence or an absence of each of the one or more conditions in the sample based at least in part on the first plurality of probabilities. In some embodiments a condition in the one or more conditions is an identification of a first species present in the first sample, and the one or more programs further comprises instructions for showing a percentage of a genome of the first species identified by the (A)(ii) in the set of sample sequence reads.

Another aspect of the present disclosure provides a computer readable storage medium storing one or more programs. The one or more programs comprising instructions, which when executed by an electronic device with one or more processors and a memory, cause the electronic device to perform a method for identifying a presence or an absence of one or more conditions in a first sample from a sample source. In some embodiments the method (i) obtains, in electronic form, a set of sample sequence reads for a plurality of polynucleotides from the first sample, wherein the set of sequence reads comprises at least 50,000 sequence reads. The method also comprises using a computer system to (ii) perform, for each respective sequence read in the set of sample sequence reads or a respective sample contig derived from a respective subset of the set of sample sequence reads, a corresponding sequence comparison between at least a portion of the respective sample sequence read or respective sample contig and each reference sequence in a first set of reference sequences, where the first set of reference sequences comprises 1000 or more reference sequences, thereby performing a first plurality of sequence comparisons. The method further comprises (iii) performing, dependent or independent of when the A(ii) is completed, for each respective sequence read in the set of sample sequence reads or a respective sample contig derived from a respective subset of the set of sample sequence reads, a corresponding sequence comparison between at least a portion of the respective sample sequence read or respective sample contig and each reference sequence in a second set of reference sequences, where the second set of reference sequences comprises 1000 or more reference sequences, thereby performing a second plurality of sequence comparisons. The method further comprises (iv) calculating, from the first and second plurality of sequence comparisons, a respective probability that the respective sample sequence read or the respective sample contig corresponds to a particular reference sequence in the first or second set of reference sequences thereby computing a first plurality of probabilities. The method further comprises (v) identifying a presence or an absence of each of the one or more conditions in the sample based at least in part on the first plurality of probabilities. In some embodiments a condition in the one or more conditions is an identification of a first species present in the first sample, and the one or more programs further comprises instructions for showing a percentage of a genome of the first species identified by (A)(ii) in the set of sample sequence reads.

Another aspect of the present disclosure provides a computer readable storage medium storing one or more programs. The one or more programs comprise instructions, which when executed by an electronic device with one or more processors and a memory, cause the electronic device to perform any of the method for identifying a presence or an absence of one or more conditions in a first sample from a sample source disclosed herein.

Additional aspects and advantages of the present disclosure will become readily apparent to those skilled in this art from the following detailed description, where only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows an example interface for an application.

FIGS. 2A-2B show example visualizations for sequencing quality control (QC) and processing control metrics, respectively.

FIG. 3 shows an example visualization for sample quality control.

FIG. 4 shows an example visualization for a quality control metric based on read length.

FIG. 5 shows an example visualization for organism identification.

FIGS. 6A-6C show example visualizations for coverage at various nucleotide positions at the gene level and at the genome level.

FIGS. 7A-7C show example visualizations for quality control failure (FIG. 7A), organisms below cutoff in the positive processing control (FIG. 7B), and additional metrics for review (FIG. 7C).

FIGS. 8A-8B show electrophoresis traces for quality control relating to adapter dimers.

FIGS. 9A-9B show example visualizations corresponding to repeat runs.

FIG. 10 shows an example visualization for quality control metrics over many sequencing runs.

FIGS. 11A-11D show example visualizations including filters for selecting species of interest (FIG. 11A), a frequency chart for organisms (FIG. 11B), a bar chart for organism types (FIG. 11C), and a bar chart showing changes in organisms over time (FIG. 11D).

FIGS. 12A-12D show an example visualization for a diagnostic test profile.

FIG. 13 shows an example visualization for switching diagnostic test profile.

FIG. 14 shows an example visualization that may allow a user to select a disease category using a graphical user interface.

FIG. 15 shows the number of publications on the web-based application user interface.

FIG. 16 shows an example of a list of publications from an external database.

FIG. 17 shows an example visualization of a filter interface.

FIG. 18 shows an example visualization of classifying organisms as members of a phylogenetically or semantically related group with the most likely organism shown at the top of the group tree view.

FIGS. 19A and 19B show an example visualization of quality control metrics.

FIG. 20 shows an example visualization of the AMR gene visualization results.

FIG. 21 shows an example visualization of information that the AMR gene visualization provides.

FIG. 22 shows an example visualization of coverage plots of an AMR gene at both protein amino acid and nucleotide levels.

FIG. 23 shows an example of NCBI record of an AMR gene reference.

FIG. 24 shows an example visualization of a detailed view of detected organisms.

FIG. 25 shows an example visualization of a BLAST query.

FIG. 26 shows an example visualization of example BLAST results.

FIG. 27 shows a computer system that is programmed or otherwise configured to implement methods of the present disclosure herein.

FIG. 28 shows an example workflow according to the methods provided herein.

FIG. 29 schematically illustrates an exemplary module-based workflow of a system.

FIG. 30 schematically illustrates an exemplary laboratory support module.

FIG. 31 schematically illustrates an exemplary quality control module.

FIG. 32 schematically illustrates an exemplary classification module.

FIG. 33 schematically illustrates an exemplary detection module.

FIG. 34 schematically illustrates an exemplary interpretation module.

FIG. 35 schematically illustrates an exemplary analytics module.

FIG. 36 schematically illustrates an exemplary commercial support module.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The systems and methods of this disclosure as described herein may employ, unless otherwise indicated, suitable techniques and descriptions of molecular biology (including recombinant techniques), cell biology, biochemistry, microarray and sequencing technology. Such techniques include polymer array synthesis, hybridization and ligation of oligonucleotides, sequencing of oligonucleotides, and detection of hybridization using a label. Specific illustrations of suitable techniques can be had by reference to the examples herein. However, equivalent procedures can, of course, also be used. Such techniques and descriptions can be found in standard laboratory manuals such as Green, et al., Eds., Genome Analysis: A Laboratory Manual Series (Vols. I-IV) (1999); Weiner, et al., Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Stryer, L., Biochemistry (4th Ed.) W.H. Freeman, N.Y. (1995); Gait, “Oligonucleotide Synthesis: A Practical Approach” IRL Press, London (1984); Nelson and Cox, Lehninger, Principles of Biochemistry, 3^{rd} Ed., W.H. Freeman Pub., New York (2000); and Berg et al., Biochemistry, 5^{th} Ed., W.H. Freeman Pub., New York (2002), all of which are herein incorporated by reference in their entirety for all purposes. Before the present compositions, research tools and systems and methods are described, it is to be understood that this disclosure is not limited to the specific systems and methods, compositions, targets and uses described, as such may, of course, vary. It is also to be understood that the terminology used herein is for the purpose of describing particular aspects only and is not intended to limit the scope of the present disclosure.

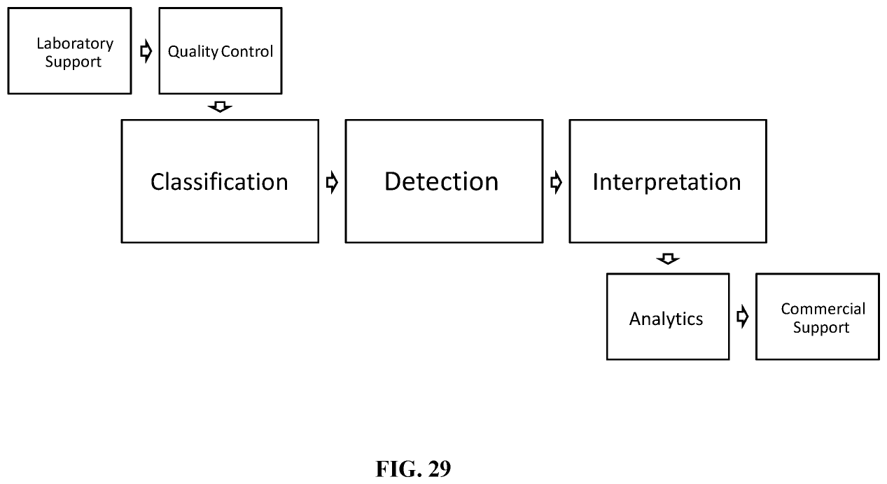

The present disclosure provides methods and systems for analyzing samples including, e.g., samples including nucleic acid molecules and/or proteins. The methods and systems of the present disclosure may facilitate identification of sequences and subsequent identification and classification of entities included within one or more samples. For example, the methods and systems provided herein may facilitate identification of microorganisms and/or pathogens within a sample, such as a cellular sample obtained from a patient. The methods of the present disclosure may comprise one or more steps including collecting a sample, processing a sample to prepare contents of the sample for sequencing analysis, performing a sequencing analysis to generate sequencing reads, processing sequencing reads to identify short sequences associated with a sample and their relationships to one another (e.g., via a k-mer based analysis, as described herein), detecting entities such as pathogens and microorganisms and/or antimicrobial resistance markers within a sample based at least in part on sequencing data, interpreting sequencing data and entity identification data, developing therapeutic or other strategies based at least in part on sequencing data and entity identification data, evaluating sequencer and classification algorithm performance, and providing a recommendation to a medical professional and/or patient or other subject. The methods and systems provided herein may comprise interaction of one or more users with an interface at one or more different times and at one or more different locations. The methods and systems provided herein may comprise one or modules, which modules may include, for example, a laboratory support module, a quality control module, a classification module, a detection module, an interpretation module, an analytics module, and a commercial support module. Such modules are schematically illustrated in FIGS. 29-36. One or more interfaces may be associated with one or more different modules. Information including sample and control information, patient information, sequencer information and protocols, suspected sample contents, sequencing data, entity classification, data reports and metrics, and any other useful information inputted into a system may be stored in any useful way, including locally, via a dedicated drive or server, or via a web- or cloud-based storage system. Information may be inputted into a system via manual or automated entry, including via scanning of text or barcodes, via accessing local or other databases, by physical transfer, by wireless or cloud-based transfer, etc. Details of methods and systems of the present disclosure are included below.

### Laboratory Support.

The present disclosure provides methods and systems for analyzing various samples. The methods and systems provided herein may include a lab module for collecting, processing, tracking, and/or displaying information regarding samples, controls, reagents, and procedures relating to the methods and systems provided herein. For example, a system may include a module configured to collect, process, retain, and display information regarding one or more samples. This module or another module may also be configured to collect, process, retain, and display information regarding one or more controls, such as a control sample including one or more known nucleic acid or amino acid sequences or one or more known microorganisms or pathogens. In some cases, the module may include information including sequence information corresponding to or derived from a database (e.g., as described herein), such as a reference database. A laboratory support module may also be configured to collect, process, retain, and display information regarding one or more laboratory procedures, such as one or more procedures useful for processing a sample (e.g., as described herein). Similarly, a laboratory support module may be configured to collect, process, retain, and display information relating to various reagents, such as reagents useful in sample processing (e.g., as described herein).

A laboratory support module may comprise or otherwise be connected to an interface through which a user may provide, view, download, or otherwise process information regarding, for example, one or more samples, controls, laboratory procedures, and/or reagents. An interface may be a web- or cloud-based interface or an application based interface on a standalone computer. An interface may be locally available via a computer or other electronic device, such as a tablet or phone. An interface may comprise via which a user may interact with other components of a system provided herein (e.g., as described herein). In an example, a user may access the interface at a first physical location and a first time at which they may provide information about one or more samples, controls, laboratory procedures, and/or reagents. The same user or another user may access the interface at a second physical location and a second time at which they may view or download such information, and/or input additional information. In an example, the user interface may be accessible via a web-based program. An interface of a laboratory support module may be shared with one or more other modules of a classification and processing system (e.g., as described herein).

Samples may originate from any useful source and may be processed in any useful way (e.g., as described herein). For example, a sample comprising nucleic acid molecules may be processed to prepare nucleic acid molecules therein for a nucleic acid sequencing assay. Alternatively or additionally, a sample comprising proteins may be processed to prepare proteins therein for a protein or amino acid sequencing assay. Controls may comprise known sequences, microorganisms, and/or pathogens, and/or may correspond to one or more databases (e.g., as described herein). Any useful processing may be used to process a sample and extract information about the sample for inputting to a user interface and use in subsequent analysis (e.g., as described herein). Similarly, any useful reagents may be used in processing of a sample. Additional details regarding samples, controls, laboratory procedures, and reagents are included below.

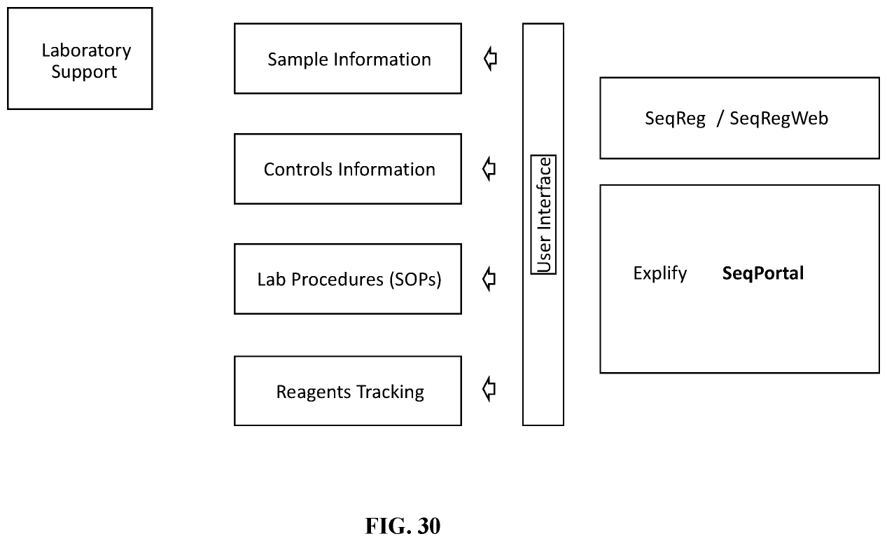

An example laboratory support module is schematically illustrated in FIG. 30.

### Samples.

The present disclosure provides methods and systems for analyzing various samples. Information regarding a sample, including its time, method, conditions, and location of collection; patient or other peripheral information, if applicable; volume; density; mass; storage container type; storage conditions; suspected contents (e.g., suspected microorganisms and/or pathogens); relevant personnel associated with the sample, including its handlers, laboratory technicians, and/or medical or other professionals authorized to access information about the sample; relevant controls; procedures used or to be used in processing the sample; reagents used or to be used in processing the sample; related samples, including other samples derived from the same source; barcode identifiers; and any other potentially useful information may be inputted into, stored by, accessed within, downloaded from, uploaded from, viewed within, processed by, and/or otherwise managed by an interface of a laboratory support module. In some cases, a sample may be deidentified such that one or more persons interacting with the sample or its associated information may be unaware of features of the sample including a patient or other source from which it is derived and/or its suspected contents.

The methods and systems provided herein may be useful for identifying microorganisms and viruses within a sample. Accordingly, the methods and systems provided herein may be useful for evaluating a sample for contamination (e.g., environmental contamination, surface contamination, food contamination, air contamination, water contamination, or cell culture contamination), stimulus response (e.g., drug responder or non-responder, allergic response, or treatment response), infection (e.g., bacterial infection, fungal infection, or viral infection), and disease state (e.g., presence or absence of disease, worsening of disease, or recovery for disease). Samples may be derived from environmental or biological sources (e.g., as described herein). The presence of microorganisms or viruses within a sample may be analyzed by, for example, analyzing nucleic acid molecules and proteins or polypeptides within the sample, such as nucleic acid molecules and proteins or polypeptides that may be derived from microorganisms or viruses. Analyzing a sample may comprise detecting sequences of nucleic acid molecules and proteins or polypeptides and comparing the sequences against sequences included in a reference database.

### Collection of Samples.

A sample may be collected from any source of interest. For example, a sample may be collected from a biological source or an environmental source. A biological source of a sample may derive from a subject, such as a mammal or other animal. The terms “subject,” “individual,” and “patient” are used interchangeably herein to refer to a vertebrate, preferably a mammal, more preferably a human. A sample may be collected from a multicellular organism such as a fish, amphibian, reptile, bird, or mammal. Mammals include, but are not limited to, murines, simians, apes, monkeys, gorillas, humans, farm animals (e.g., cows, pigs, sheep, horses), rodents (e.g., rats, mice), sport animals, and pets (e.g., cats, dogs, rabbits). For example, a subject may be a human. A sample may be collected from a population of microbes, and/or from a cell line. For example, a sample may be collected from chromalveolata such as malaria, and dinoflagellates. Tissues, cells, and their progeny of a biological entity obtained in vivo or cultured in vitro are also encompassed.

A subject may have or be suspected of having a disease or disorder. A subject may be known to have previously had a disease or disorder. A subject may have been or be suspected of having been exposed to a pathogen such as a virus or bacteria. A subject may have a risk factor for a given disease. A subject may be healthy or believed to be healthy. A subject may have a given characteristic, such as a given weight, height, body mass index, or other characteristic. A subject may have a given ethnic or racial heritage, place of birth or residence, nationality, disease or remission state, family medical history, or other characteristic. A subject may be or have spent time in a given location, such as a medical facility or office, hospital, laboratory, or clinic. For example, a subject may be or have spent time in a hospital where they may be suspected of having been exposed to a pathogen. A subject may use or have used (e.g., have implanted or inserted) a medical device such as a catheter, bandage, stent, needle, cannula, breast pump, tube (e.g., tympanostomy tube), hearing aid, prosthetic, defibrillator, artificial hip, artificial knee, pacemaker, implant (e.g., breast implant), screws, rods, stitches, discs (e.g., spinal discs), intrauterine device, pins, plates, or eye lens. For example, a subject may have or have previously had an inserted catheter. A medical device may provide a mechanism for exposure of a subject to a pathogen (e.g., via formation of a biofilm).

As used herein, the term “biological sample” is used interchangeably with the term “sample” and generally refers to a sample obtained from a subject. The biological sample may be obtained directly or indirectly from the subject. A sample may be obtained from a subject via any suitable method, including, but not limited to, spitting, swabbing, blood draw, biopsy, obtaining excretions (e.g., urine, stool, sputum, vomit, or saliva), excision, scraping, and puncture. A sample may be obtained from a subject by, for example, intravenously or intraarterially accessing the circulatory system, collecting a secreted biological sample (e.g., stool, urine, saliva, sputum, etc.), breathing, or surgically extracting a tissue (e.g., biopsy). The sample may be obtained by non-invasive methods including but not limited to: scraping of the skin or cervix, swabbing of the cheek, or collection of saliva, urine, feces, menses, tears, or semen. Alternatively, the sample may be obtained by an invasive procedure such as biopsy, needle aspiration, or phlebotomy. A sample may comprise a bodily fluid such as, but not limited to, blood (e.g., whole blood, red blood cells, leukocytes or white blood cells, platelets), plasma, serum, sweat, tears, saliva, sputum, urine, semen, mucus, synovial fluid, breast milk, colostrum, amniotic fluid, bile, bone marrow, interstitial or extracellular fluid, lymphatic fluid, peritoneal effusion, pleural effusion, aqueous humor, bursa fluid, eye wash, eye aspirate, pulmonary lavage, lung aspirate, buffy coat, or cerebrospinal fluid. For example, a sample may be obtained by a puncture method to obtain a bodily fluid comprising blood and/or plasma. Such a sample may comprise both cells and cell-free nucleic acid material. Alternatively, the sample may be obtained from any other source including but not limited to blood, sweat, hair follicle, buccal tissue, tears, menses, feces, or saliva. The biological sample may be a tissue sample or chemical treated tissue sample, such as a tumor biopsy. The sample may be obtained from any of the tissues provided herein including, but not limited to, skin, heart, lung, kidney, breast, pancreas, liver, intestine, brain, prostate, esophagus, muscle, smooth muscle, bladder, gall bladder, colon, or thyroid. The methods of obtaining provided herein include methods of biopsy including fine needle aspiration, core needle biopsy, vacuum assisted biopsy, large core biopsy, incisional biopsy, excisional biopsy, punch biopsy, shave biopsy or skin biopsy. The biological sample may comprise one or more cells.

A sample may comprise cells of a primary culture or a cell line. Examples of cell lines include, but are not limited to 293-T human kidney cells, A2870 human ovary cells, A431 human epithelium, B35 rat neuroblastoma cells, BHK-21 hamster kidney cells, BR293 human breast cells, CHO chinese hamster ovary cells, CORL23 human lung cells, HeLa cells, or Jurkat cells. The sample may comprise a homogeneous or mixed population of microbes, including one or more of viruses, bacteria, protists, monerans, chromalveolata, archaea, or fungi. Examples of viruses include, but are not limited to human immunodeficiency virus, ebola virus, rhinovirus, influenza, rotavirus, hepatitis virus, West Nile virus, ringspot virus, mosaic viruses, herpesviruses, lettuce big-vein associated virus. Non-limiting examples of bacteria include Staphylococcus aureus, Staphylococcus aureus Mu3; Staphylococcus epidermidis, Streptococcus agalactiae, Streptococcus pyogenes, Streptococcus pneumonia, Escherichia coli, Citrobacter koseri, Clostridium perfringens, Enterococcus faecalis, Klebsiella pneumonia, Lactobacillus acidophilus, Listeria monocytogenes, Propionibacterium granulosum, Pseudomonas aeruginosa, Serratia marcescens, Bacillus cereus. Yersinia enterocolitica. Staphylococcus simulans, Micrococcus luteus, and Enterobacter aerogenes. Examples of fungi include, but are not limited to, Absidia corymbifera, Aspergillus niger, Candida albicans, Geotrichum candidum, Hansenula anomala, Microsporum gypseum, Monilia, Mucor, Penicilliusidia corymbifera, Aspergillus niger, Candida albicans, Geotrichum candidum, Hansenula anomala, Microsporum gypseum, Monilia, Mucor, Penicillium expansum, Rhizopus, Rhodotorula, Saccharomyces bayabus, Saccharomyces carlsbergensis, Saccharomyces uvarum, and Saccharomyces cerivisiae. A sample can also be a processed sample such as a preserved, fixed and/or stabilized sample.

A sample may be collected from an environmental source. For example, a sample may be collected from a field (e.g., an agricultural field), lake, river, creek, ocean, watershed, water tank, water reservoir, pool (e.g., swimming pool), pond, air vent, wall, roof, soil, plant, or other environmental source. Collection of a sample from an environmental source may comprise collecting water, soil, or air in, e.g., one or more containers, such as a vial or pipette. Collection of a sample from an environmental source may comprise contacting water or soil with a wicking or adhesive material. Collection of a sample from an environmental source may comprise swabbing a surface.

A sample may be collected from an industrial source. Industrial sources include, for example, clean rooms (e.g., in manufacturing or research facilities), hospitals, medical laboratories, pharmacies, pharmaceutical compounding centers, pharmaceutical production materials and facilities, food processing areas, food production areas, water or waste treatment facility, and food stuffs. For example, one or more pieces of equipment in a medical facility may be a source for collection of a sample. A waiting or consultation area in a medical facility may also be a source for collection of a sample. Collection of a sample from an industrial source may comprise swabbing a surface or contacting a surface with a wicking or adhesive material.

Collection of a sample may comprise air or water sampling. For example, a sample may be collected from ambient air in a facility (e.g., a medical facility or other facility). A sample may be collected from a subject, such as by collecting exhaled or expectorated air from the subject. An air sample may comprise biological contaminants in the air as aerosols. Such contaminants may include bacteria, fungi, viruses, and pollens. Aerosols may be solid or liquid particles suspended in air and may vary in size from, e.g., less than about 100 microns (μm), such as less than about 50 μm, 25 μm, 12 μm, 10 μm, 5 μm, 1 μm, 500 nanometers (nm), 200 nm, 100 nm, or smaller. Particles may consist of a single, unattached organism or may occur clustered with other material, such as with other organisms, dust, organic material, or inorganic material. Particles suspended in air may become oxidized the longer they remain suspended in air and, as a result, may grow in size. Vegetative forms of bacterial cells and viruses may be present in the air in a lesser number than bacterial or fungal spores. Microorganisms within a bioaerosol may be alive or may not be alive. For example, suspending media, relative humidity, temperature, oxygen sensitivity, and exposure to electromagnetic radiation may influence survival of microorganisms in air. Particles from air may settle onto surfaces.

Air sampling may be affected by factors including temperature, time of day, time of year, relative humidity, number and characteristics of visitors to a facility, indoor traffic, relative concentration of particles or organisms, and performance of air-handling system components. When analyzing air samples, multiple samples may be collected from a same or similar sites, such as at the same or different times. Collection of multiple samples may facilitate obtaining accurate and precise analysis of microorganisms and viruses within the samples. Air sampling may comprise use of a vacuum pump and an airflow measuring device such as an anemometer or flowmeter. Air sampling may comprise impingement in liquids (e.g., drawing air through a small jet and directing it against a liquid surface), impaction on solid surfaces (e.g., drawing air into sampler and depositing particles on a dry surface), sedimentation (e.g., particles settle onto surfaces via gravity), filtration (e.g., air drawn through a filtration mechanism and particles of a desired size trapped), centrifugation (e.g., aerosols subjected to centrifugal force and impacted onto a solid surface), electrostatic precipitation (e.g., air drawn over an electrostatically charged surface and particles become charged), thermal precipitation (e.g., air drawn over a thermal gradient and particles repelled from hot surfaces to settle on colder surfaces), or a combination thereof.

Collection of a sample may comprise sampling of a liquid such as water. Water sampling may be performed to detect waterborne pathogens of clinical significance or to determine the quality of water in a facility. For example, water sampling may be used to assess contamination in dialysis systems in medical facilities. Microorganisms in a liquid sample may be alive or may not be alive. Microorganisms in treated water may be stressed. Water sampling may comprise adding one or more chemicals to a water source, e.g., to alter the pH of the water. For example, a reducing agent such as sodium thiosulfate may be added to water to neutralize residual chlorine or other materials in a sample. A chelating agent may be added to chelate metals in a water sample. A liquid (e.g., water) sample may be combined with a media configured to affect the growth or health of microorganisms within the sample, such as a recovery media that may be a nutrient rich media. Water collected from a tap may be collected after flushing of a water line. In an example, water may be collected from a tap, and attachments to a faucet from which the water is collected may be removed and analyzed in parallel. Collecting a water sample may comprise collecting at least 100 milliliters of water, in one or more containers. Collection of a water sample may comprise the use of plates such as aerobic, heterotrophic plates. Water may be filtered or otherwise processed prior to collection of the sample (e.g., to remove bulky contaminants including dirt and plant particles).

Collection of a sample may comprise environmental surface sampling. A sample may be collected from a surface before or after a sterilization or disinfecting process. For example, a sample may be collection from a surface after a sterilization or disinfecting process to confirm the effectiveness of the sterilization or disinfecting procedure. Sample collection may proceed by contacting a surface with a swab, sponge, wipe, agar surface, or membrane filter, any of which may be moistened prior to contacting the surface. A neutralizing chemical may be used to target disinfectant ingredients where applicable. Methods of environmental-surface sampling include contacting a surface with a moistened swab, sponge, or wipe and rinsing the collecting tool; direct immersion; containment; and replicate organism direct agar contact.

A sample may be collected by a technician (e.g., a laboratory or medical technician), nurse, doctor, healthcare worker, industry worker, health and safety specialist, or any other practitioner. A sample may be collected by an individual from the individual, such as by swabbing a component of the individual's oral cavity or providing sputum or saliva in a container. A sample collected by an individual may be provided to a medical or laboratory facility for analysis. A sample may be collected from a subject in a medical facility such as a doctor office, dialysis center, or hospital.

A sample may be contacted with a media to preserve or enhance microorganisms and viruses included therein. A sample may be contacting with a material e.g., to facilitate its collection. For example, a sample may be contacted with peptone or buffered peptone water, phosphate buffered saline, sodium chloride, ringer solution (e.g., Calgon ringer or thiosulfate ringer solutions), tryptic soy broth, brain-heart infusion broth, or another material. A sample collected onto a material, such as a sample collected from a surface, may be subjected to elution, agitation, ultrasonic bath, centrifugation, or other processing to remove material from a sampling device and break up any clumps (e.g., clumps of organisms) that may be included therein.

A sample may be collected into or transferred into a container such as a vial. A sample may be reconstituted with water or a media such as a nutrient-rich media. A sample may be divided amongst a plurality of containers. For example, a sample may be divided into a plurality of containers such that sample included within different containers may be subjected to different analyses, used as controls, stored for later use, or otherwise processed. A sample may be divided immediately upon collection or after storage and/or transfer of the sample (e.g., from a collection site). A sample may be transferred under frozen or refrigerated or cold or room temperature conditions.

### Contents of Samples.

A sample may comprise a plurality of materials. As described above, a sample may be processed to remove various contaminants or deactivate contaminants including metals, large agglomerates or other materials, and chemical contaminants.

A sample may comprise one or more microorganisms or viruses or parasites. One or more microorganisms or viruses of a sample may be commonly associated with the sample source and may not be considered to be harmful. For example, hundreds of microorganisms are known to co-exist in the oral microbiome, and their existence in a sample collected from the oral cavity of a subject may not be indicative of a disease state. Such microorganisms may exist in a symbiotic (e.g., endosymbiotic) relationship with a host organism. One or more microorganisms within a sample may be considered “healthy” or “normal” microorganisms, or may even be considered beneficial to health, such as probiotics. Various microorganisms may contribute to immune health, synthesize useful vitamins, or ferment indigestible carbohydrates. Alternatively or in addition, one or more microorganisms or viruses of a sample may be associated with a disease or may be otherwise harmful to a population, such as a human population. For example, a microorganism or virus may be a pathogen that may be a causative agent in an infectious disease. Such microorganisms and viruses may be included in a sample at an acceptable level (e.g., at a level unlikely to induce disease or infection in a subject or group of subjects). Taxonomy may be used to classify microorganisms and viruses identified using the methods and systems provided herein (e.g., as described herein).

A sample may comprise one or more cells or tissues. Alternatively, a sample may be substantially cell-free. A sample that is not a cell-free sample may be processed to provide a cell-free sample. A cell-free sample may be derived from any source (e.g., as described herein), such as tissue, blood, sweat, urine, or saliva. A “cell-free sample,” as used herein, generally refers to a sample that is substantially free of cells (e.g., less than 10% cells on a volume basis).

A sample may comprise one or more proteins or polypeptides. A protein included in a sample may be initially provided in a tertiary or quaternary structure. Alternatively, a protein included in a sample may be provided in a primary or secondary structure, e.g., as a result of partial or complete denaturation of the protein (e.g., upon contacting the sample with a denaturing agent). A protein may be included within a cell or tissue. Alternatively, a protein may not be included within a cell or tissue.

The terms “polypeptide”, “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. An amino acid may be proteinogenic or non-proteinogenic. Examples of proteinogenic amino acids include arginine, histidine, lysine, aspartic acid, glutamic acid, serine, threonine, asparagine, glutamine, cysteine, selenocysteine, glycine, proline, alanine, isoleucine, leucine, methionine, phenylalanine, tryptophan, tyrosine, valine, selenocysteine, or pyrrolysine. A proteinogenic amino acid may be a genetically encoded amino acid that may be incorporated into a protein during translation. A non-proteinogenic amino acid may be a naturally occurring amino acid or a non-naturally occurring amino acid. Non-proteinogenic amino acids include amino acids that are not found in proteins and/or are not naturally encoded or found in the genetic code of an organism. Examples of non-proteinogenic amino acids include, but are not limited to, hydroxyproline, selenomethionine, hypusine, 2-aminoisobutyric acid, αγ-aminobutyric acid, ornithine, citrulline, β-alanine (3-aminopropanoic acid), δ-aminolevulinic acid, 4-aminobenzoic acid, dehydroalanine, carboxyglutamic acid, pyroglutamic acid, norvaline, norleucine, alloisoleucine, t-leucine, pipecolic acid, allothreonine, homocysteine, homoserine, α-amino-n-heptanoic acid, α,β-diaminopropionic acid, α,γ-diaminobutyric acid, β-amino-n-butyric acid, β-aminoisobutyric acid, isovaline, sarcosine, N-ethyl glycine, N-propyl glycine, N-isopropyl glycine, N-methyl alanine, N-ethyl alanine, N-methyl β-alanine, N-ethyl β-alanine, isoserine, and α-hydroxy-γ-aminobutyric acid.

A sample may comprise one or more nucleic acid molecules such as one or more deoxyribonucleic acid (DNA) and/or ribonucleic acid (RNA) molecules (e.g., included within cells or not included within cells). A sample can comprise or consist essentially of RNA. A sample can comprise or consist essentially of DNA. Nucleic acid molecules may be included within cells. Alternatively or in addition to, nucleic acid molecules may not be included within cells (e.g., cell-free nucleic acid molecules). Cell-free polynucleotides may be extracellular polynucleotides present in a sample (e.g. a sample from which cells have been removed, a sample that is not subjected to a lysis step, or a sample that is treated to separate cellular polynucleotides from extracellular polynucleotides). For example, cell-free polynucleotides include polynucleotides released into circulation upon death of a cell, and may be isolated as cell-free polynucleotides from a plasma fraction of a blood sample.

The term “nucleic acid molecule” may be used interchangeably with the terms “polynucleotide”, “nucleotide sequence”, “nucleic acid,” “nucleic acid fragment,” and “oligonucleotide” herein. They generally refer to a polymeric form of nucleotides of any length, such as deoxyribonucleotides (dNTPs) or ribonucleotides (rNTPs), or analogs thereof. Polynucleotides may have any three-dimensional structure, and may perform any function, known or unknown. A nucleic acid molecule may have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. An oligonucleotide is typically composed of a specific sequence of four nucleotide bases: adenine (A); cytosine (C); guanine (G); and thymine (T) (uracil (U) for thymine (T) when the polynucleotide is RNA). Oligonucleotides may include one or more nonstandard nucleotide(s), nucleotide analog(s) and/or modified nucleotides. Non-limiting examples of polynucleotides include deoxyribonucleic acid (DNA), genomic DNA, ribonucleic acid (RNA), cell-free DNA (e.g., cfDNA), synthetic DNA/RNA, coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

A nucleic acid may be a target nucleic acid or sample nucleic acid. A target nucleic acid may be amplified to generate an amplified product. A target nucleic acid may be, for example, a target DNA or a target RNA. A target nucleic acid may be provided in a biological sample.

A nucleic acid molecule is comprised of a plurality of nucleotides. During a sequencing procedure, nucleotides may be provided to a nucleic acid template for incorporation, and detection of incorporation events used to determine a sequence of the nucleic acid template (e.g., as described herein). The term “nucleotide,” as used herein, generally refers to a substance including a base (e.g., a nucleobase), sugar moiety, and phosphate moiety. A nucleotide may comprise a free base with attached phosphate groups. A substance including a base with three attached phosphate groups may be referred to as a nucleoside triphosphate. When a nucleotide is being added to a growing nucleic acid molecule strand, the formation of a phosphodiester bond between the proximal phosphate of the nucleotide to the growing chain may be accompanied by hydrolysis of a high-energy phosphate bond with release of the two distal phosphates as a pyrophosphate. The nucleotide may be naturally occurring or non-naturally occurring (e.g., a modified or engineered nucleotide).

The term “nucleotide analog,” as used herein, may include, but is not limited to, a nucleotide that may or may not be a naturally occurring nucleotide. For example, a nucleotide analog may be derived from and/or include structural similarities to a canonical nucleotide such as adenine-(A), thymine-(T), cytosine-(C), uracil-(U), or guanine-(G) including nucleotide. A nucleotide analog may comprise one or more differences or modifications relative to a natural nucleotide. Examples of nucleotide analogs include inosine, diaminopurine, 5-fluorouracil, 5-bromouracil, 5-chlorouracil, 5-iodouracil, hypoxanthine, xanthine, deazaxanthine, deazaguanine, isocytosine, isoguanine, 4-acetylcytosine, 5-(carboxyhydroxylmethyl)uracil, 5-carboxymethylaminomethyl-2-thiouridine, 5-carboxy methylaminomethyluracil, dihydrouracil, beta-D-galactosylqueosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5″-methoxy carboxy methyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids, and modified versions thereof (e.g., by oxidation, reduction, and/or addition of a substituent such as an alkyl, hydroxyalkyl, hydroxyl, or halogen moiety). Nucleic acid molecules (e.g., polynucleotides, double-stranded nucleic acid molecules, single-stranded nucleic acid molecules, primers, adapters, etc.) may be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety, or phosphate backbone. In some cases, a nucleotide may include a modification in its phosphate moiety, including a modification to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates), and modifications with selenium moieties (e.g., phosphoroselenoate nucleic acids). A nucleotide or nucleotide analog may comprise a sugar selected from the group consisting of ribose, deoxyribose, and modified versions thereof (e.g., by oxidation, reduction, and/or addition of a substituent such as an alkyl, hydroxyalkyl, hydroxyl, or halogen moiety). A nucleotide analog may also comprise a modified linker moiety (e.g., in lieu of a phosphate moiety). Nucleotide analogs may also contain amine-modified groups, such as aminoallyl-dUTP (aa-dUTP) and aminohexylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure may provide, for example, higher density in bits per cubic mm, higher safety (resistant to accidental or purposeful synthesis of natural toxins), easier discrimination in photo-programmed polymerases, and/or lower secondary structure. Nucleotide analogs may be capable of reacting or bonding with detectable moieties for nucleotide detection (e.g., during a sequencing process, as described herein).

### Controls.

The methods and systems provided herein may comprise the preparation, use, or processing of one or more controls. A control may be collected in the same or different manner as a sample (e.g., as described herein) and may include similar or different contents. For example, a sample and a control may be collected in the same manner and from a same source at the same or different times. The sample may be subjected to a first processing protocol while the control may be subjected to a second processing protocol that is different from the first, or may not undergo any substantial processing. Alternatively or additionally, a control may be prepared from and/or include one or more known entities. For example, a control may comprise one or more known microorganisms and/or pathogens; in some embodiments, this type of control may serve as an external control. In some embodiments, an internal control may be included to ensure the assay works and all the reagents demonstrate proper function. A control can be processed separately from a sample, or a control can be added into a sample and processed together with the sample. A sample and the control may be subjected to parallel processing and comparison between information obtained regarding the sample and control may be used to determine whether the sample includes the same one or more known microorganisms and/or pathogens, and/or to assess a laboratory or computational process. For example, a control may include a first microorganism and a second microorganism, and a sample may be suspected of including one or both of the first and second microorganism. The control and sample may be subjected to parallel processing using the same methods, reagents, and computational protocols to identify microorganisms included therein. Successful identification and optional quantification of the first and second microorganisms within the control may indicate that the methods and systems used to process the sample and control are capable of effectively processing a sample to identify a microorganism included therein. Similarly, unsuccessful identification and/or optional quantification of the first and/or second microorganisms, such as identification of only a single microorganism of the first and second microorganisms or incorrect quantification of a microorganism, within the control may indicate that the methods and/or systems used to process the sample and control require calibration, threshold adjustment, improved database curation, or another improvement. Successful identification and optional quantification of the first and second microorganisms within the control may also be useful in identifying and/or quantifying a given microorganism within the sample.

One or more controls may be used for comparison with a given sample. For example, a single control may be interrogated in parallel with a given sample or set of samples. Alternatively, multiple controls may be interrogated in parallel with a given sample or set of samples. For example, multiple controls including multiple different known sequences or entities or combinations thereof may be used.

In some embodiments, 10 or more controls, 100 or more controls, 1000 or more controls, 10,000 or more controls, 100,000 or more controls, or 1×10⁶ or more controls, each control representing a different known sequence, are used.

In an example, a sample suspected of including a first entity and a second entity (e.g., a first microorganism and a second microorganism) may be interrogated in parallel with a control known to include the first entity and the second entity, or nucleic acid or amino acid sequences thereof. Alternatively or additionally, a sample suspected of including a first entity and a second entity (e.g., a first microorganism and a second microorganism) may be interrogated in parallel with a first control known to include the first entity or a nucleic acid or amino acid sequence thereof and a second control known to include the second entity or a nucleic acid or amino acid sequence thereof. Alternatively or additionally, a sample suspected of including a first entity and a second entity (e.g., a first microorganism and a second microorganism) may be interrogated in parallel with a first control known to include the first entity or a nucleic acid or amino acid sequence thereof, a second control known to include the second entity or a nucleic acid or amino acid sequence thereof, and a third control known to include the first entity and the second entity, or nucleic acid or amino acid sequences thereof. Alternatively or additionally, a sample suspected of including a first entity and a second entity (e.g., a first microorganism and a second microorganism) may be interrogated in parallel with a first control known to include the first entity and the second entity, or nucleic acid or amino acid sequences thereof, and a second control known to not include the first entity or the second entity, or nucleic acid or amino acid sequences thereof.

A control may comprise a physical sample that is processed and analyzed (e.g., as described herein). Alternatively or additionally, a control may comprise a control data set comprising a control set of nucleic acid and/or amino acid sequences. For example, a control may comprise a control set of nucleic acid sequences, amino acid sequences, and/or weighted k-mers associated with a control set of nucleic acid or amino acid sequences (e.g., as described herein), which sequences and/or weighted k-mers may correspond to one or more known entities, such as one or more microorganisms. In some embodiments the control set is a control set of nucleic acid sequences and comprises 10 or more nucleic acid sequences, 100 or more nucleic acid sequences, 1000 or more nucleic acid sequences, 10,000 or more nucleic acid sequences, 100,000 or more nucleic acid sequences, or 1×10⁶ or more nucleic acid sequences. In some embodiments the control set is a control set of amino acid sequences and comprises 10 or more amino acid sequences, 100 or more amino acid sequences, 1000 or more amino acid sequences, 10,000 or more amino acid sequences, 100,000 or amino nucleic acid sequences, or 1×10⁶ or more amino acid sequences. In some embodiments the control set is a control set of weighted k-mers and comprises 1000 or more weighted k-mers, 10,000 or more weighted k-mers, 100,000 or more weighted k-mers, 1×10⁶ or more weighted k-mers, 1×10⁷ or more weighted k-mers, or 1×10⁸ or more weighted k-mers. Such a data set may have been experimentally derived, e.g., by a user. For example, a user may have prepared and processed a control sample to provide a control comprising a control data set comprising a known set of nucleic acid and/or amino acid sequences, and/or weighted k-mers associated with a known set of nucleic acid and/or amino acid sequences. Alternatively or additionally, a control comprising such a data set may be derived from one or more reference databases (e.g., as described herein).

Information regarding a control may be inputted to a system provided herein via an interface (e.g., as described herein). Alternatively, information regarding a control may be downloaded, uploaded, or otherwise accessed from another source. For example, information regarding a control may be obtained from a database (e.g., as described herein) and/or otherwise provided to a system such as a laboratory support module. Information regarding a control may be inputted into, stored by, accessed within, downloaded from, uploaded from, viewed within, processed by, and/or otherwise managed by an interface, such as an interface of a laboratory support module. Information regarding a control may include, e.g., its time, method, conditions, and location of collection and/or preparation; patient or other peripheral information, if applicable; volume; density; mass; storage container type; storage conditions; suspected or known contents (e.g., suspected or known microorganisms and/or pathogens); relevant personnel associated with the control, including its handlers, laboratory technicians, and/or medical or other professionals authorized to access information about the sample; relevant samples; procedures used or to be used in processing the control; reagents used or to be used in processing the control; related samples, including other samples and/or controls derived from the same source; relevant databases; barcode identifiers; and any other potentially useful information. In some cases, a control may be deidentified such that one or more persons interacting with the sample or its associated information may be unaware of features of the sample including a patient or other source from which it is derived and/or its suspected contents.

### Sample Processing.

A sample may be subjected to one or more processes prior to analysis as provided herein. Procedures for processing one or more samples may be inputted into, stored by, accessed within, downloaded from, uploaded from, viewed within, processed by, and/or otherwise managed by an interface, such as an interface of a laboratory support module. Such procedures may include standard operating procedures applicable to processing of various samples from one or more different sources. For example, such procedures may comprise sample collection procedures for collection of samples from patients. Procedures may further relate to sample storage and regulation; transfer of samples between one or more different locations and/or between one or more different containers; isolation of nucleic acid molecules, proteins, and/or cells or enrichment of the same within a sample or derivative thereof; sample purification; amplification of sequences; nucleic acid sequencing and/or protein sequencing; information storage; or any other aspect relating to collection and subsequent processing of a sample. Such procedures may be accessible by personnel involved with the collection and/or processing of samples. For example, a procedure for collecting a sample may be accessible by personnel tasked with collecting a sample from a source such as a patient. Alternatively or additionally, one or more procedures may be accessible to one or more different personnel. For example, procedures relating to nucleic acid sequencing and preparation therefore may be accessible to laboratory technicians who are separate from personnel tasked with collection and initial preparation and storage of a sample. One or more procedures may be accessible by any user of a laboratory support module of a system provided herein.

In some cases, one or more procedures may be selectable by a user and set as a default procedure for one or more aspects of processing of a sample. For example, a user such as a doctor or laboratory technician at a medical facility may select one or more procedures relating to, e.g., sample collection, storage, and processing, which one or more procedures may be providable to technicians and/or other personnel tasked with carrying out such processes. Such procedures may be set such that only a designated user or type of user may alter them. This may help ensure uniform collection and handling of samples by one or more different personnel and/or from one or more different sources. The same user or another use may select one or more procedures relating to further processing of samples. In an example, a first user may select a procedure or set of procedures relating to sample collection, storage, and, optionally, initial processing. Such a procedure or set of procedures may also include protocols for inputting, storing, and/or deidentifying samples. The procedure or set of procedures may relate to particular sample type, patient type, and/or suspected entities within a sample. For example, the procedure or set of procedures may be specific to samples suspected of containing a particular entity, such as a staphylococcus bacterium or other pathogen. Alternatively or additionally, the procedure or set of procedures may be specific to samples deriving from a particular source, such as samples comprising blood. Additional procedures may be selected and/or established for different sample types, patient types, and/or suspected entities within a sample. A second user, who may be the same or different than the first user, may select a procedure or set of procedures relating to processing of samples. Such a procedure or set of procedures may relate to, for example, preparing for and performing one or more sequencing assays to provide sequencing reads relating to entities included within a given sample. Such a procedure or set of procedures may be carried out by a different set or class of personnel, and optionally at a different location and/or different time. For example, a first set of personnel may carry out sample collection and initial processing according to a first set of procedures established by a first user, and a second set of personnel may carry out further sample processing according to a second set of procedures established by the same or a different user. The different sets of procedures may be carried out at one or more different locations, including at one or more different locations within a medical facility such as a hospital. Additional procedures, including procedures relating to analysis and interpretation of data output, may be set and/or carried out by different combinations of users and personnel.

A procedure for processing a sample may relate to storage and/or transfer of a sample. For example, a sample may be stored for a period of time subsequent to its collection. A sample may be stored in any useful vessel, for any useful time, and under any useful conditions. A sample may be stored for, e.g., at least 1 hour, such as at least about 2 hours, 4 hours, 6 hours, 10 hours, 12 hours, 24 hours, 48 hours, 72 hours, 1 week, or longer. A sample may be stored in the container into which it is collected or initially provided. Alternatively, a sample may be transferred to one or more different containers for storage. A sample may be stored at room temperature. Alternatively or in addition, a sample may be stored in an incubator or in a refrigerator or freezer system. For example, a biological sample (e.g., a blood sample) may be stored in a refrigerator or freezer until it may be analyzed. For example, a sample may be stored at a temperature of at most about 15° C., 10° C., 5° C., 0° C., −5° C., or lower.

A sample may be prepared by combining a first material (e.g., as described herein) and a second material. The first and second materials may be collected from a subject or source (e.g., a same subject or source) at the same or different times. Alternatively or in addition, a sample collected from a subject or source may be subdivided into two or more portions (e.g., for analysis at different times or via different processes).

A sample may undergo one or more processes including, for example, purification, extraction, filtration, selective precipitation, permeabilization, isolation, heating, agitation, or centrifugation. One or more such processes may be performed prior to subjecting the sample to storage and/or analysis as provided herein. Alternatively or in addition, one or more such processes may be performed after the samples has been stored for a period of time, and optionally before storage of the sample for an additional period of time. A sample may be processed to remove agglomerates and/or to de-agglomerate clumps of microorganisms and viruses. For example, a sample may be undergo one or more filtration, agitation, or centrifugation processes to process clumps or aggregates included therein. A sample may be reconstituted with a material or media configured to affect the survival of microorganisms therein, such as a growth media. Alternatively, a sample may be combined with a material configured to kill microorganisms therein. A sample may be combined with one or more materials to preserve or alter an aspect of the sample, such as a preservative, buffer, or detergent.

A sample may be transferred between containers prior to, during, or subsequent to storage or any processing described herein. For example, a sample may be aliquoted to provide a plurality of samples for one or more different analyses. A sample may be transported from a collection site to a storage site, a processing site, and/or an analysis site, any of which may be the same or different. For example, a sample may be collected at a first site and transferred to a second site different from the first site for analysis. In another example, a sample may be collected in a facility such as a medical facility, optionally stored, and eventually analyzed in the same facility. Collection and analysis in a same facility may facilitate precise, accurate, and rapid detection of materials included within a sample.

A sample may be deidentified prior to, during, or subsequent to any processing, and optionally before undergoing analysis as provided herein. Deidentification of a sample may comprise obfuscation of identifying information of a sample, such as a subject or source from which it is collected, or details thereof; time of collection; site of collection; or other details. This may be performed by assigning a sample an identifying code such as a barcode or QR code. Information linking the identifying information of the sample and the identifying code may be retained in a database. The database may be configured to be inaccessible to all or some users to ensure that identifying information of samples is not readily available to users. Deidentification of samples may help ensure that samples are analyzed without preconceived ideas of what they may or may not contain, and may also help protect confidentiality for subjects (e.g., patients) in a medical setting.

Preparing a sample for analysis according to the methods provided herein may comprise lysing or permeabilizing cells (e.g., by contacting a sample with a lysing or permeabilizing agent), degrading tissues, and denaturing proteins and nucleic acid molecules (e.g., by contacting a sample with a denaturing agent such as a detergent). Sample preparation may also comprise extracting nucleic acid molecules and/or polypeptides within samples. For example, sample preparation may comprise contacting the sample with an agent configured to degrade a lipid envelope and/or protein coat (e.g., capsid) of a virus to provide access to genetic material therein. A sample may be divided prior to such preparation to provide a first aliquot and a second aliquot, which first and second aliquots may undergo parallel but different processing. For example, the first aliquot may undergo processing to extract and preserve nucleic acid molecules, while the second aliquot may undergo processing to extract and preserve polypeptides.

### Preparation for Nucleic Acid Sequencing.

A procedure for processing a sample or portion thereof may relate to nucleic acid sequencing. For example, the sample may be processed to extract nucleic acid molecules from cells and viruses and identify nucleic acid sequences associated with the same. Nucleic acid sequencing may be carried out at any useful facility using any useful method and by any useful personnel.

A variety of methods may be used to extract and/or purify nucleic acid molecules of a sample. For example, nucleic acids can be purified using an organic extraction method. Other non-limiting examples of extraction techniques include: (1) organic extraction followed by ethanol precipitation, e.g., using a phenol/chloroform organic reagent with or without the use of an automated nucleic acid extractor, e.g., the Model 341 DNA Extractor available from Applied Biosystems (Foster City, Calif.); (2) stationary phase adsorption methods; and (3) salt-induced nucleic acid precipitation methods, such precipitation methods being typically referred to as “salting-out” methods. Another example of nucleic acid isolation and/or purification includes the use of magnetic particles to which nucleic acids can specifically or non-specifically bind, followed by isolation of the beads using a magnet, and washing and eluting the nucleic acids from the beads. An isolation method may be preceded by an enzyme digestion step to help eliminate unwanted protein from the sample, e.g., digestion with proteinase K, or other like proteases. If desired, RNase inhibitors may be added to a lysis buffer. For certain cell or sample types, it may be desirable to add a protein denaturation/digestion step to the protocol. Purification methods may be directed to isolate DNA, RNA, or both. When both DNA and RNA are isolated together during or subsequent to an extraction procedure, further steps may be employed to purify one or both separately from the other. Sub-fractions of extracted nucleic acids can also be generated, for example, purification by size, sequence, or other physical or chemical methods.

Nucleic acid molecules may be contacted with one or more adapters or primers to prepare nucleic acid molecules for an amplification and/or sequencing process. As used herein, the terms “adaptor” and “adapter” are used interchangeably and generally refer to an oligonucleotide that may be attached to an end of a nucleic acid. Adaptor sequences may comprise, for example, priming sites, the complement of a priming site, recognition sites for endonucleases, common sequences, promoters, barcode sequences, sequencing primers, and flow cell attachment sequences. Adaptors may also incorporate modified nucleotides that modify the properties of the adaptor sequence. For example, phosphorothioate groups may be incorporated in one of the adaptor strands. An adaptor may be double-stranded or single-stranded. For example, an adapter coupled to a single nucleic acid strand may be a single-stranded adaptor, while an adapter coupled to a double-stranded nucleic acid molecule may be a double-stranded adapter. An adaptor may have any useful length. For example, an adaptor may have at least 2, 4, 6, 8, 10, 12, 14, 16, 18, 20, or more nucleotides (e.g., in a given strand). A nucleic acid molecule may include a first adaptor at a first end and a second adapter at a second end. For example, a double-stranded nucleic acid molecule may include a first adaptor at a first end and a second adaptor at a second end, where the first adaptor and second adaptor include identical nucleic acid sequences (e.g., on opposite strands). An adapter may be coupled to a nucleic acid molecule in various ways, such as by ligation (e.g., blunt end ligation) or hybridization. An adapter may be configured to facilitate amplification of a nucleic acid molecule in a nucleic acid amplification reaction. Alternatively or in addition, an adapter may be configured to facilitate sequencing in a sequencing reaction (e.g., an adapter may comprise a flow cell or sequencing adapter).

Nucleic acid molecules of a sample may undergo amplification or target enrichment procedures prior to a sequencing reaction to increase the detectable population of nucleic acid molecules within the sample. Alternatively, nucleic acid molecules of a sample may not be amplified prior to undergoing sequencing. The terms “amplifying.” “amplification.” and “nucleic acid amplification” are used interchangeably herein and generally refer to generating one or more copies of a nucleic acid or a template. For example, “amplification” of DNA generally refers to generating one or more copies of a DNA molecule. An amplicon may be a single-stranded or double-stranded nucleic acid molecule that is generated by an amplification procedure from a starting template nucleic acid molecule (e.g., target nucleic acid molecule). Such an amplification procedure may include one or more cycles of an extension or ligation procedure. The amplicon may comprise a nucleic acid strand, of which at least a portion may be substantially identical or substantially complementary to at least a portion of the starting template. Where the starting template is a double-stranded nucleic acid molecule, an amplicon may comprise a nucleic acid strand that is substantially identical to at least a portion of one strand and is substantially complementary to at least a portion of either strand. The amplicon can be single-stranded or double-stranded irrespective of whether the initial template is single-stranded or double-stranded. Amplification of a nucleic acid may linear, exponential, or a combination thereof. Amplification may be emulsion based or may be non-emulsion based. Non-limiting examples of nucleic acid amplification methods include reverse transcription, primer extension, polymerase chain reaction (PCR), ligase chain reaction (LCR), helicase-dependent amplification, bridge amplification, template walking/wildfire amplification, nanoball-based amplification, asymmetric amplification, rolling circle amplification, and multiple displacement amplification (MDA), nucleic acid hybridization capture-based enrichment. Where PCR is used, any form of PCR may be used, with non-limiting examples that include real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR and touchdown PCR. Moreover, amplification can be conducted in a reaction mixture comprising various components (e.g., a primer(s), template, nucleotides, a polymerase, buffer components, co-factors, etc.) that participate or facilitate amplification. In some embodiments, the reaction mixture comprises a buffer that permits context independent incorporation of nucleotides, such as, for example, magnesium-ion, manganese-ion and isocitrate buffers. Amplification may be clonal amplification. Clonal amplification may provide concentrated populations of nucleic acid molecules comprising identical sequences.

In an example, a multiplexed PCR process may be used to amplify a nucleic acid molecule. An amplification process may comprise Multiplex Biotinylated Asymmetric PCR. The methods may enable simultaneous sequencing of thousands of regions of interest corresponding to nucleic acid molecules from a nucleic acid sample. Sensitivity to detect low amounts of targets in a sample is driven by Multiplex PCR, while subsequent Asymmetric PCR provides increased specificity. Logical partitioning and directionality considerations may be used to facilitate these processes. Such methods may allow for high through put sequencing of various target sequences without requiring the use of ligation or enzymatic digestion methods. Examples of such amplification methods are described in at least PCT/US2018/060915, which is herein incorporated by reference in its entirety.

Amplification may involve the use of a polymerase. The term “polymerase” or “polymerizing enzyme,” as used herein, generally refers to any enzyme capable of catalyzing a polymerization reaction. A polymerase may be used to extend a nucleic acid primer coupled to a template nucleic acid strand by incorporation of nucleotides or nucleotide analogs. A polymerase may extend a nucleic acid strand by extending, e.g., the 3′ end of an existing nucleotide chain, adding new nucleotides matched to the template strand one at a time via the creation of phosphodiester bonds. A polymerase may have strand displacement activity or non-strand displacement activity. A polymerase may be a nucleic acid polymerase. A polymerase may have high processivity (e.g., ability to consecutively incorporate nucleotides into a nucleic acid template without releasing the nucleic acid template). A polymerase may be capable of incorporating modified nucleotides and dideoxynucleotide triphosphates. A polymerase may have a modified nucleotide binding, which may be useful for nucleic acid sequencing. Examples of polymerases include, but are not limited to, a DNA polymerase, an RNA polymerase, a thermostable polymerase, a wild-type polymerase, a modified polymerase, E. coli DNA polymerase I, T7 DNA polymerase, bacteriophage T4 DNA polymerase Φ29 (phi29) DNA polymerase, Taq polymerase, Tth polymerase, Tli polymerase, Pfu polymerase, Pwo polymerase, VENT polymerase, DEEPVENT polymerase, EX-Taq polymerase, LA-Taq polymerase, Sso polymerase, Poc polymerase, Pab polymerase, Mth polymerase, ES4 polymerase, Tru polymerase, Tac polymerase, Tne polymerase, Tma polymerase, Tea polymerase, Tih polymerase, Tfi polymerase, Platinum Taq polymerases, Tbr polymerase, Tfl polymerase, Pfu-turbo polymerase, Pyrobest polymerase, Pwo polymerase, KOD polymerase, Bst polymerase, Sac polymerase, Klenow fragment, polymerase with 3′ to 5′ exonuclease activity, and variants, modified products and derivatives thereof. A polymerase may be, e.g., a Family A or Family B polymerase. Examples of Family A polymerases include, but are not limited to, Taq, Klenow, and Bst polymerases. Examples of Family B polymerases include, but are not limited to, Vent(exo-) and Therminator polymerases.

Nucleotides and nucleotide analogs (e.g., as described herein) may be used in nucleic acid amplification reaction. For example, nucleic acid molecules may be amplified using canonical nucleotides, modified nucleotides (e.g., nucleotide analogs), or a combination thereof.

Coupling of adapters to nucleic acid molecules and/or nucleic acid amplification may rely on sequence complementarity and/or may generate nucleic acid strand comprising complementary sequences. The term “complementarity,” as used herein, generally refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively).

“Perfectly complementary” means that all the contiguous residues of a nucleic acid sequence will hydrogen bond with the same number of contiguous residues in a second nucleic acid sequence. “Substantially complementary.” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. The term “complementary sequence,” as used herein, generally refers to a sequence that hybridizes to another sequence. Hybridization between two single-stranded nucleic acid molecules may involve the formation of a double-stranded structure that is stable under certain conditions. Two single-stranded polynucleotides may be considered to be hybridized if they are bonded to each other by two or more sequentially adjacent base pairings. A substantial proportion of nucleotides in one strand of a double-stranded structure may undergo Watson-Crick base-pairing with a nucleoside on the other strand. Hybridization may also include the pairing of nucleoside analogs, such as deoxyinosine, nucleosides with 2-aminopurine bases, and the like, that may be employed to reduce the degeneracy of probes, whether or not such pairing involves formation of hydrogen bonds. Sequence identity, such as for the purpose of assessing percent complementarity, may be measured by any suitable alignment algorithm, including but not limited to the Needleman-Wunsch algorithm (see e.g. the EMBOSS Needle aligner available at www.ebi.ac.uk/Tools/psa/emboss_needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/nucleotide.html, optionally with default settings). Optimal alignment may be assessed using any suitable parameters of a chosen algorithm, including default parameters.

An amplification process may be performed in a solution. Amplification may be performed while nucleic acid molecules are immobilized to a surface, such as a surface of a particle or surface (e.g., chip or flow cell). Alternatively or in addition, amplification may be performed in compartments, such as wells or droplets (e.g., emulsion PCR). Amplification may be performed within a sequencing instrument. Alternatively, amplification may be performed prior to provision of amplified nucleic acid molecules to a sequencing instrument.

### Preparation for Protein Sequencing.

A procedure for processing a sample or portion thereof may relate to protein sequencing. For example, the sample may be processed to extract proteins from cells and viruses and identify polypeptide and/or amino acid sequences associated with the same. Protein sequencing may be carried out at any useful facility using any useful method and by any useful personnel.

A sample comprising a protein may be subjected to an Edman degradation process to prepare the protein for sequencing using an Edman sequencer process. An Edman sequencer may be capable of sequencing peptide fragments of approximately 50 amino acids or longer. The preparation process may comprise contacting the solution comprising the protein with a reducing agent such as 2-mercaptoethanol to break disulfide bridges. A protecting group (e.g., iodoacetic acid) may be provided to prevent reformation of bonds. Individual chains of a protein may be separated and purified and the amino acid composition of each chain may be determined. The terminal amino acids of each chain may also be determined. Each chain may be broken into fragments, such as fragments under 50 amino acids long. The fragments may be separated and purified. The sequences of each fragment may be determined. This process may be repeated with a different pattern of cleavage and subsequently the sequence of the overall protein may be constructed.

Protein sequencing may comprise isolation of a protein within a sample, such as using sodium dodecyl sulfate-polyacrylamide gel electrophoresis (SDS-PAGE) or chromatography. The isolated protein may be chemically modified to stabilize various residues such as cysteine residues. The protein may be digested (e.g., with one or more proteases such as trypsin) to generate a plurality of peptides. The peptides may be desalted to remove ionizable contaminants. Peptides may then be subjected to sequencing processes (e.g., as described herein).

### Sequence Identification.

A procedure for processing a sample may relate to identification of a sequence of a nucleic acid molecule and/or protein included within the sample or a derivative thereof. Sequences of nucleic acid molecules and proteins may be identified to determine the presence or absence of, e.g., microorganisms and viruses within a sample. Identifying sequences of nucleic acid molecules and proteins may comprise performance of one or more sequencing processes.

The terms “nucleic acid sequencing” and “sequencing.” as used herein, generally refers to a process for generating or identifying a sequence of a biological molecule, such as a nucleic acid molecule or a polypeptide. Such sequence may be a nucleic acid sequence, which may include a sequence of nucleic acid bases (e.g., nucleobases). A sequence may be a polypeptide sequence, which may be a sequence of amino acids. Sequencing may be, for example, single molecule sequencing, sequencing by synthesis, sequencing by hybridization, or sequencing by ligation. Sequencing may be performed using template nucleic acid molecules immobilized on a support, such as a flow cell or one or more beads. A sequencing assay may yield one or more sequencing reads corresponding to one or more template nucleic acid molecules. Sequencing a polypeptide may comprise, for example, an Edman degradation process, de novo sequencing, mass spectrometric analysis, or a combination thereof.

The term “sequence identity,” as used herein, generally refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (e.g., polynucleotide or amino acid sequences) can be compared by determining their “percent identity.” to one another. The percent identity of two sequences, whether nucleic acid or amino acid sequences, is the number of exact matches between two aligned sequences divided by the length of the shorter sequences and multiplied by 100. Percent identity may also be determined, for example, by comparing sequence information using a database or program such as the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (e.g., nucleotides or amino acids), divided by the total number of symbols in the shorter of the two sequences. The program may be used to determine percent identity over the entire length of the proteins being compared. Default parameters may be provided to optimize searches with short query sequences in, for example, with the BLASTp program. The program also allows use of an SEG filter to mask-off segments of the query sequences as determined by the SEG program of Wootton and Federhen, Computers and Chemistry 17:149-163 (1993). Ranges of desired degrees of sequence identity may be approximately 80% to 100% and integer values therebetween (e.g., about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%). In general, an exact match indicates 100% identity over the length of the shortest of the sequences being compared (or over the length of both sequences, if identical).

Prior to performing a sequence process, a sample may divided into one or more portions. For example, a sample may be divided into a first portion for nucleic acid processing and a second portion for polypeptide sequencing. The first and/or second portions may be further subdivided to provide additional sample aliquots for control, storage, and/or additional analysis.

Nucleic acid and protein sequencing may provide complementary information.

For example, nucleic acid sequencing may provide insight into what genes may be expressed by a cell or organism and what proteins may be produced. Similarly, protein sequencing may provide insight into mRNA that may have been included in a given cell or organism. As used herein, “expression” generally refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, the term “differentially expressed,” as applied to nucleotide sequence or polypeptide sequence in a subject, generally refers to over-expression or under-expression of that sequence when compared to that detected in a control. Underexpression also encompasses absence of expression of a particular sequence as evidenced by the absence of detectable expression in a test subject when compared to a control.

As used herein, a “control” generally refers to an alternative subject or sample used in an experiment for comparison purpose.

Sequencing information may be collected for a single sample or a plurality of samples. For example, sequencing information may be collected for a plurality of samples at a same time or at different times. Sequencing information collected for a plurality of samples combined for data processing, optionally after associating the sequencing information for each different sample with an identifying code. Multiple samples can be sequenced at the same time and processed and differentiated by different identifiers, or multiple samples can be sequenced in the same sequencing process but loaded at different times.

### Sequencing of Nucleic Acid Molecules.

Nucleic acid molecules of a sample may interrogated to determine their nucleic acid sequences. Nucleic acid sequences of, for example, DNA and RNA may be used to identify a source from which they derive, such as a virus or microorganism from which they derive. Nucleic acid sequences identified within a sample may be compared against sequences within a database to associate them with the source from which they derive (e.g., as described herein).

Nucleic acid sequencing may be performed on a sample or portion thereof that has undergone a nucleic acid amplification process. Alternatively, sequencing may be performed on a sample or portion thereof that has not undergone a nucleic acid amplification process. Nucleic acid molecules within a sample or portion thereof may be fragmented prior to undergoing sequencing. Alternatively, nucleic acid molecules may not be fragmented prior to undergoing sequencing. Multiple different schemes may be applied to identify nucleic acid sequences within a sample.

Different types of nucleic acid molecules may undergo the same or different processing and sequencing. For example, DNA molecules may undergo a first sequencing process and RNA molecules may undergo a second sequencing process, where the first and second sequencing processes may include at least one process difference. In an example, genomic DNA such as accessible chromatin is processed according to a first sequencing method (e.g., using an assay for transposase-accessible chromatin using sequencing (ATAC-seq) method) while RNA molecules are processed according to a second sequencing method (e.g., a sequencing method that targets RNA molecules that include a polyA sequence, such as messenger RNA (mRNA) molecules). Different sequencing procedures may be performed on the same or different samples. For example, a first sequencing method to analyze a first type of nucleic acid molecule and a second sequencing method to analyze a second type of nucleic acid molecule, where the first and second sequencing methods are different and the first and second types of nucleic acid molecules are different, may be performed on a same sample (e.g., at the same or different times). Alternatively or in addition, a first sequencing method to analyze a first type of nucleic acid molecule may be performed using a first sample and a second sequencing method to analyze a second type of nucleic acid molecule may be performed using a second sample, where the first and second sequencing methods are different, the first and second types of nucleic acid molecules are different, and the first and second samples are different. The first and second samples may be aliquots of a same sample (e.g., as described herein).

Nucleic acid sequencing may be quantitative or approximately quantitative. Alternatively, nucleic acid sequencing may be qualitative and may not provide significant insight into the relative amounts of different nucleic acid molecules included within a sample.

Various sequencing schemes may be employed. For example, sequencing by synthesis, sequencing by hybridization, sequencing by ligation, nanopore sequencing, sequencing using nucleic acid nanoballs, pyrosequencing, single molecule sequencing (e.g., single molecule real time sequencing), single cell/entity sequencing, massively parallel signature sequencing, polony sequencing, combinatorial probe anchor synthesis, SOLID sequencing, chain termination (e.g., Sanger sequencing), ion semiconductor sequencing, tunneling currents sequencing, heliscope single molecule sequencing, sequencing with mass spectrometry, transmission electron microscopy sequencing. RNA polymerase-based sequencing, or any other method, or a combination thereof, may be used. Sequencing technologies like Heliscope (Helicos), SMRT technology (Pacific Biosciences) or nanopore sequencing (Oxford Nanopore) may allow direct sequencing of single molecules without prior clonal amplification. Sequencing may be performed with or without target enrichment. Sequencing may be performed within a solution. Sequencing may be performed with nucleic acid molecules immobilized (e.g., directly or indirectly) to a substrate. Sequencing may be performed within a microfluidic device. Sequencing may comprise consensus sequencing.

Sequencing may comprise Helicos True Single Molecule Sequencing (tSMS) (e.g. as described in Harris et al., Science 320: 106-109 [2008]). In a typical tSMS process, a DNA sample is cleaved into strands of approximately 100 to 200 nucleotides, and a polyA sequence is added to the 3′ end of each DNA strand. Each strand is labeled by the addition of a fluorescently labeled adenosine nucleotide. The DNA strands are then hybridized to a flow cell, which contains millions of oligo-T capture sites that are immobilized to the flow cell surface. The templates can be at a density of about 100 million templates/cm². The flow cell is then loaded into an instrument, e.g., HeliScope™ sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are discerned by imaging the flow cell surface. After imaging, a cleavage step removes the fluorescent label, and the process is repeated with other fluorescently labeled nucleotides until the desired read length is achieved. Sequence information is collected with each nucleotide addition step.

Another example process for sequencing polynucleotides is 454 sequencing (Roche) (e.g. as described in Margulies et al. Nature 437:376-380 (2005)). In a first step, DNA is typically sheared into fragments of approximately 300-800 base pairs, and the fragments are blunt-ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is discerned and analyzed.

A further example of suitable DNA sequencing technology is the SOLIDTY, technology (Applied Biosystems). In SOLIDTY sequencing-by-ligation, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

DNA sequencing may be by single molecule, real-time (SMRT™) sequencing technology of Pacific Biosciences. In SMRT sequencing, the continuous incorporation of dye-labeled nucleotides is imaged during DNA synthesis. Single DNA polymerase molecules are attached to the bottom surface of individual zero-mode wavelength identifiers (ZMW identifiers) that obtain sequence information while phospholinked nucleotides are being incorporated into the growing primer strand. A ZMW is a confinement structure that enables observation of incorporation of a single nucleotide by DNA polymerase against the background of fluorescent nucleotides that rapidly diffuse in an out of the ZMW (in microseconds). It takes several milliseconds to incorporate a nucleotide into a growing strand. During this time, the fluorescent label is excited and produces a fluorescent signal, and the fluorescent tag is cleaved off. Identification of the corresponding fluorescence of the dye indicates which base was incorporated. The process may be repeated.

Sequencing may also comprise nanopore sequencing (e.g. as described in Soni G V and Meller A. Clin Chem 53: 1996-2001 [2007]). Nanopore sequencing DNA analysis techniques are being industrially developed by a number of companies, including Oxford Nanopore Technologies (Oxford, United Kingdom). Nanopore sequencing is a single-molecule sequencing technology whereby a single molecule of DNA is sequenced directly as it passes through a nanopore. A nanopore may be a small hole, of the order of 1 nanometer in diameter. Immersion of a nanopore in a conducting fluid and application of a potential (voltage) across it results in a slight electrical current due to conduction of ions through the nanopore. The amount of current which flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore represents a reading of the DNA sequence.

Sequencing may comprise the use of a chemical-sensitive field effect transistor (chemFET) array (see e.g. US20090026082). In one example of the technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be discerned by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

Sequencing may comprise Ion Torrent single molecule sequencing, which pairs semiconductor technology with a simple sequencing chemistry to directly translate chemically encoded information (A, C, G, T) into digital information (0, 1) on a semiconductor chip. In nature, when a nucleotide is incorporated into a strand of DNA by a polymerase, a hydrogen ion is released as a byproduct. Ion Torrent uses a high-density array of micro-machined wells to perform this biochemical process in a massively parallel way. Each well holds a different DNA molecule. Beneath the wells is an ion-sensitive layer and beneath that an ion sensor. When a nucleotide, for example a C, is added to a DNA template and is then incorporated into a strand of DNA, a hydrogen ion may be released. The charge from that ion may change the pH of the solution, which can be identified by Ion Torrent's ion sensor. The sequencer calls the base, going directly from chemical information to digital information. The Ion personal Genome Machine (PGM™) sequencer then sequentially floods the chip with one nucleotide after another. If the next nucleotide that floods the chip is not a match. No voltage change may be recorded and no base may be called. If there are two identical bases on the DNA strand, the voltage may be double, and the chip may record two identical bases called. Direct identification allows recordation of nucleotide incorporation in seconds.

A sequencing process may comprise detecting a signal such as a fluorescent signal (e.g., an emission signal from a fluorescent label) with a detector. The term “detector,” as used herein, generally refers to a device that is capable of detecting or measuring a signal, such as a signal indicative of the presence or absence of an incorporated nucleotide or nucleotide analog. A detector may include optical and/or electronic components that may detect and/or measure signals. Non-limiting examples of detection methods involving a detector include optical detection, spectroscopic detection, electrostatic detection, and electrochemical detection. Optical detection methods include, but are not limited to, fluorimetry and UV-vis light absorbance. Spectroscopic detection methods include, but are not limited to, mass spectrometry, nuclear magnetic resonance (NMR) spectroscopy, and infrared spectroscopy. Electrostatic detection methods include, but are not limited to, gel-based techniques, such as, for example, gel electrophoresis. Electrochemical detection methods include, but are not limited to, electrochemical detection of amplified product after high-performance liquid chromatography separation of the amplified products.

In some embodiments, sequence reads are acquired by any methodology known in the art. For example, next generation sequencing (NGS) techniques such as sequencing-by-synthesis technology (Illumina), pyrosequencing (454 Life Sciences), ion semiconductor technology (Ion Torrent sequencing), single-molecule real-time sequencing (Pacific Biosciences), sequencing by ligation (SOLID sequencing), nanopore sequencing (Oxford Nanopore Technologies), or paired-end sequencing can be used. In some embodiments, massively parallel sequencing is performed using sequencing-by-synthesis with reversible dye terminators. In some embodiments, sequencing is performed using next generation sequencing technologies, such as short-read technologies. In other embodiments, long-read sequencing or another sequencing method known in the art is used.

Next-generation sequencing produces millions of short reads (e.g., sequence reads) for each biological sample. Accordingly, in some embodiments, the plurality of sequence reads obtained by next-generation sequencing of nucleic acid molecules are DNA sequence reads. In some embodiments, the sequence reads have an average length of at least fifty nucleotides. In other embodiments, the sequence reads have an average length of at least 50, 60, 70, 80, 90, 100, 150, 200, 250, 300, or more nucleotides.

In some embodiments, sequencing is performed after enriching for nucleic acids (e.g., cfDNA, gDNA, and/or RNA) encompassing a plurality of predetermined target sequences, e.g., human genes and/or non-coding sequences associated with a condition such as cancer. Advantageously, sequencing a nucleic acid sample that has been enriched for target nucleic acids, rather than all nucleic acids isolated from a biological sample, significantly reduces the average time and cost of the sequencing reaction. Accordingly, in some embodiments, the methods described herein include obtaining a plurality of sequence reads of nucleic acids that have been hybridized to a probe set for hybrid-capture enrichment.

In some embodiments, panel-targeting sequencing is performed to an average on-target depth of at least 30×, at least 40×, at least 50×, at least 60×, at least 70×, at least 80×, at least 90×, at least 100×, at least 500×, at least 750×, at least 1000×, at least 2500×, at least 500×, at least 10,000×, or greater depth. In some embodiments, samples are further assessed for uniformity above a sequencing depth threshold (e.g., 95% of all targeted base pairs at 300× sequencing depth). In some embodiments, the sequencing depth threshold is a minimum depth selected by a user or practitioner. In some embodiments, the panel-targeting sequencing includes probes for between two and 1000 genomic regions, between 500 and 5,000 genomic regions, between 1,000 and 20,000 genomic regions or between 5,000 and 50,000 genomic regions.

In some embodiments, the sequence reads are obtained by a whole genome sequencing methodology. In some such embodiments, the whole genome sequencing is performed at lower sequencing depth than smaller target-panel sequencing reactions, because many more loci are being sequenced. For example, in some embodiments, whole genome sequencing is performed to an average sequencing depth of at least 0.2×, at least 0.5×, at least 1×, at least 1.5×, at least 2×, at least 2.5×, at least 3×, at least 3.5×, at least 4×, at least 4.5×, or greater. In some embodiments, whole genome sequencing is performed to an average sequencing depth of no more than 7.5×, no more than 7×, no more than 6.5×, no more than 6×, no more than 5.5×, no more than 5×, no more than 4.5×, no more than 4×, no more than 3.5×, no more than 3×, no more than 2.5×, no more than 2×, no more than 1.5×, no more than 1×, or less. In some embodiments, low-pass whole genome sequencing (LPWGS) is performed to an average sequencing depth of about 0.25× to about 5×, or to an average sequencing depth of about 0.5× to about 5×, or to an average sequencing depth of about 1× to about 5×, or to an average sequencing depth of about 2× to about 5×, or to an average sequencing depth of about 3× to about 5×, or to an average sequencing depth of about 1× to about 4×, or to an average sequencing depth of about 1× to about 3×, or to an average sequencing depth of about 1.5× to about 4×, or to an average sequencing depth of about 1.5× to about 3×, or to an average sequencing depth of about 2× to about 3×.

In some embodiments, 100 or more sequence reads, 1000 or more sequence reads, 10,000 or more sequence reads, 20,000 or more sequence reads, 30,000 or more sequence reads, 40,000 or more sequence reads, 50,000 or more sequence reads, 60,000 or more sequence reads, 70,000 or more sequence reads, 80,000 or more sequence reads, 90,000 or more sequence reads, 100,000 or more sequence reads, 110,000 or more sequence reads, 120,000 or more sequence reads, 130,000 or more sequence reads, 140,000 or more sequence reads, 150,000 or more sequence reads, 160,000 or more sequence reads, 170,000 or more sequence reads, 180,000 or more sequence reads, 190,000 or more sequence reads, 200,000 or more sequence reads, 300,000 or more sequence reads, 400,000 or more sequence reads, 500,000 or more sequence reads, 1×10⁶ or more sequence reads, 1×10⁷ or more sequence reads, or 1×10⁸ or more sequence reads are obtained from a biological sample. In some such embodiments, each sequence read has a minimum length. In some embodiments, this minimum length is 10, 11, 12, 13, 14, 15, 16, 17, 18, 19, 20, 25, 30, 35, 40, 45, 50, or more residues. In some embodiments each sequence read has a maximum length. In some embodiments this maximum length is a number between 400 residues and 1000 residues. In some embodiments, each sequence length has a maximum length of 500, 600, 700, 800, 900, or 1000 residues.

### Sequencing of Proteins.

Protein molecules of a sample may be interrogated to determine their protein sequences. Protein sequences may be used to identify a source from which they derive, such as a virus or microorganism from which they derive. Protein sequences identified within a sample may be compared against sequences within a database to associate them with the source from which they derive (e.g., as described herein).

Protein molecules within a sample or portion thereof may be fragmented prior to undergoing sequencing. Alternatively or in addition, protein molecules may not be fragmented prior to undergoing sequencing. Multiple different schemes may be applied to identify protein sequences within a sample.

Different types of protein molecules may undergo the same or different processing and sequencing. For example, protein molecules having a first size or characteristic may undergo a first sequencing process and protein molecules having a second size or characteristic may undergo a second sequencing process, where the first and second sequencing processes may include at least one process difference. Different sequencing procedures may be performed on the same or different samples. For example, a first sequencing method to analyze a first type of protein molecule and a second sequencing method to analyze a second type of protein molecule, where the first and second sequencing methods are different and the first and second types of protein molecules are different, may be performed on a same sample (e.g., at the same or different times). Alternatively or in addition, a first sequencing method to analyze a first type of protein molecule may be performed using a first sample and a second sequencing method to analyze a second type of protein molecule may be performed using a second sample, where the first and second sequencing methods are different, the first and second types of protein molecules are different, and the first and second samples are different. The first and second samples may be aliquots of a same sample (e.g., as described herein).

Protein sequencing may be quantitative or approximately quantitative. Alternatively, protein sequencing may be qualitative and may not provide significant insight into the relative amounts of different protein molecules included within a sample.

Various sequencing schemes may be employed. For example, protein sequencing may comprise an Edman degradation process. Protein sequencing may comprise sequencing protein fragments and/or whole polypeptides. Fragmenting may be cleared using different mechanisms to produce overlapping fragments. As described herein, fragments and whole polypeptides may be separated and purified prior to sequencing. Protein sequencing may comprise mass spectrometric analysis (e.g., matrix-assisted laser desorption/ionization-time of flight (MALDI-TOF) mass spectrometry). In some cases, direct measurement of peptide masses may provide sufficient information to identify the protein. Additional fragmentation (e.g., within the mass spectrometer) may provide further insight into peptide sequences. Peptides may alternatively be desalted and separated by reverse phase high performance liquid chromatography (HPLC) coupled to a mass spectrometer, e.g., using an electrospray ionization source (ESI). Fragmentation of peptides may proceed via mechanisms such as collision-induced dissociation or post-source decay. Measured mass to charge ratios may be compared to calculated mass values from, e.g., in silico proteolysis and fragmentation of databases of protein sequences and matched based on exact sequence identity or similarity to homologous proteins. Alternatively or in addition, de novo sequencing may be used to analyze protein sequences. Whole mass analysis of a protein (e.g., un-fragmented protein) may also be performed by subjecting an un-fragmented protein to, e.g., ESI-mass spectrometry. This mechanism may be sufficient to confirm the termini of the protein and infer the presence or absence of various post-translational modifications.

### Reagents.

As described herein, one or more different reagents may be used in processing a sample or collection of samples. For example, a first reagent or set of reagents may be used in a first procedure for processing a sample and second reagent or set of reagents may be used in a second procedure for processing the sample. Reagents may also be included in a sample as buffers, stabilizers, detergents, cryoprotectants, or for any other useful purpose. Reagents may also be used to enrich any targeted nucleic acid sequences.

The types, amounts, sources, and other details of reagents may be predetermined by one or more users. Such information may be included with procedures selected for use in processing a sample (e.g., as described herein). Information regarding a reagent may be inputted to a system provided herein via an interface (e.g., as described herein). Alternatively, information regarding a reagent may be downloaded, uploaded, or otherwise accessed from another source. For example, information regarding a reagent may be obtained from a database (e.g., as described herein) and/or otherwise provided to a system such as a laboratory support module. Information regarding a reagent may be inputted into, stored by, accessed within, downloaded from, uploaded from, viewed within, processed by, and/or otherwise managed by an interface, such as an interface of a laboratory support module. Information regarding a reagent may include, e.g., its time, method, conditions, and location of preparation; volume; density; mass; safety information; storage container type; storage conditions; suspected contaminants; relevant personnel associated with the reagent; relevant sample types; relevant procedures; barcode identifiers; and any other potentially useful information. Different reagents and protocols relating to their use may be tracked from, e.g., purchase or manufacture through their eventual use and replenishment by the same or different personnel. For example, a first set of reagents used in a first set of procedures may be tracked separately from a second set of reagents used in a second set of procedures, such as a second set of procedures performed by different personnel and/or at a different location or time. Different sets of reagents may include the same reagents. For example, first and second sets of reagents may each include a given reagent, which reagent may be tracked within each grouping and/or independently.

### Barcodes.

As used herein, the term “barcode” refers to a label, or identifier, that conveys or is capable of conveying information (e.g., information about a sequence read. A barcode can be part of an analyte, or independent of an analyte. A barcode can be attached to a sequence read. In some embodiments, a barcode encodes a unique predetermined value selected from the set {1, . . . , 1024}. {1, . . . , 4096}, {1, . . . , 16384}, {1, . . . , 65536}, {1, . . . , 262144}, {1, . . . , 1048576}, {1, . . . , 4194304}, {1, . . . , 16777216}, {1, . . . , 67108864}, or {1, . . . , 1×10¹²}.

### Quality Control

The methods and systems provided herein also provide mechanisms for monitoring the quality of various processes. For example, the methods and systems provided herein may comprise a quality control module configured to track and/or evaluate the effectiveness of a method or system at identifying and/or quantifying an entity or collection of entities within a sample. Quality control methods may comprise the use of one or more controls (e.g., as described herein), which one or more controls may be processed at least partially in parallel to one or more samples.

In some cases, the performance of a sequencer may be monitored. Sequencer performance monitoring may provide, for example, inputting a control comprising one or more known entities or sequences thereof into a sequencing instrument, performing a sequencing procedure, and evaluating the resultant sequencing reads to determine whether a sequencer and corresponding sequencing process can precisely and accurately identify the known entities or sequences within the control. Evaluation of sequencer performance may comprise evaluating the sequencer and/or sequencing procedure's ability to effectively quantify one or more known entities or sequences thereof within a control. Evaluation of a sequencer may comprise inputting a given control or set of controls into the sequencer regularly (e.g., before and/or after a sample run or during a sample run). For example, one or more controls may be used to evaluate a sequencer on a regular basis, such as hourly, daily, weekly, or monthly. Alternatively or additionally, one or more controls may be used to evaluate a sequencer before, during, or after processing of a sample, such as immediately before or after processing a sample, or within 24 hours of processing a sample. Different controls may be evaluated to assess different sensitivities of a sequencer. For example, a first control comprising a first set of known entities or sequences thereof may be used to evaluate a sequencer prior to, during, or subsequent to analysis of a sample suspected of including an entity of the first set of known entities, while a second control comprising a second set of known entities or sequences thereof may be used to evaluate a sequencer prior to, during, or subsequent to analysis of a sample suspected of including an entity of the second set of known entities. Running controls before, during, or after processing of one or more samples may ensure the quality of a sequencing run.

Sequencing quality may be evaluated based on one or more different metrics. For example, accuracy and precise identification of specific sequences and their prevalence within a sample or control may be evaluated. Error rates, quality scores (including Phred quality scores), and other metrics may also be used to evaluate sequencing quality.

In some cases, evaluating quality of a sequencing run may comprise, e.g., demultiplexing and adaptor trimming processes, read quality filtering, read quality trimming, and evaluation of reads subsequent to one or more of such processes. In some cases, evaluation of quality of a sequencing run may involve evaluation of input libraries, which may in turn provide feedback for performance of various sample preparation (e.g., laboratory performance) procedures.

In some cases, sequencing data including sequencing reads prepared using, e.g., next-generation sequencing (e.g., as described herein) may undergo an initial quality assessment prior to being subjected to a classification process. For example, sequencing data may be processed to assess the quality of the underlying sequencing libraries prepared in the laboratory to improve the quality of base calls. Analysis of reads in Fastqs for factors such as sequence diversity, base call Phred quality scores (Q), and presence of adaptor sequences may provide insight into the performance of library preparations. Poorer quality reads, such as those having more than half of calls with Q<20, may be filtered out. Adaptor sequences may be trimmed from sequence ends, as may be poorer quality base calls that have Q<30. Following this filtering and trimming, remaining reads and base calls in sequencing data (e.g., in fastq files) may be quantitatively rated by assigning a Sample Quality Score. This Score may help inform the reliability of a diagnostic result, especially in cases where library preparations may have been challenging due to the nature of a clinical sample such as high viscosity or low cellularity.

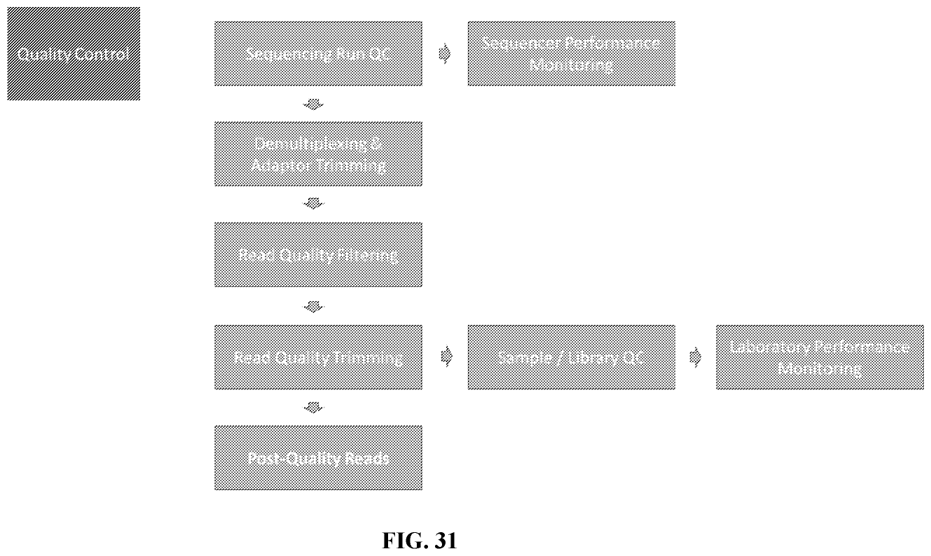

An example quality control module is schematically illustrated in FIG. 31.

### Classification.

Identification and classification of one or more entities and/or sequences thereof within a sample may comprise various processes including, for example, nucleic acid sequencing and/or protein sequencing. For example, classification of an entity may comprise identification and optional quantification of sequence associated with the entity via nucleic acid sequencing. Identification of a sequence within a sample may in some cases not immediately identify an entity within the sample. For example, multiple different entities may include the sequence (e.g., the sequence may be common to a grouping of entities) or a sequence with high sequence homology, the sequence may be included in a short or fragmented read, etc. The abundance of known and unknown microorganisms and pathogens is such that a detailed sequence analysis may be required to accurately identify an entity within a sample. Such an analysis may comprise identification of short sequence segments within broader sequence reads and performing a probabilistic analysis comparing the sequence against one or more curated databases to identify a given sequence as being associated with a particular entity or class of entity.

Identification of sequences within a given sample or control and classification of entities within the given sample or control may be performed within a classification module. A classification module may comprise one or more elements with which a user may interact, including, for example, a display or user interface. A classification module may be operatively linked to an interface through which sequencing read and/or sample and control information may be inputted, stored, viewed, accessed, downloaded, manipulated, or uploaded. A user may interact with an interface prior to, during, and/or subsequent to a classification process. For example, a user may view; establish, and/or update thresholds for analysis; select or view analysis protocols; and select or view reference databases; select, manipulate, view, hide, or otherwise interact with reports or other outputs. A classification module may comprise a display component via which one or more users may view reports or other outputs, including species identification and treatment recommendations. The display may be incorporated into a user interface and may have any useful features.

A classification module may perform operations locally, in a cloud, via web, via one or more servers, or any combination thereof. In an example, sample information and sequencing reads may be locally inputted at a first location to a web-based storage system, and sequence analysis and classification may subsequently be performed over a network. A user may monitor and provide input to the sequence analysis and classification processes as they are performed via a web-based user interface at a second location. Classification may comprise, for example, read k-merization, data binning, preparation and/or accessing reference databases, sequence assembly (e.g., via k-mer analysis, exact sequence matching, other sequence identification processes, and consensus sequencing), and read alignments, among other processes.

A classification process may begin with filtered and trimmed sequencing data (e.g., in the form of fastq files) as inputs. Initially, a binning process may assign reads to broad categories of organisms, such as bacteria, fungi, parasite, and virus, as well as host (for example, human). A classification algorithm may then compare each set of binned reads to reference sequences that correspond to an assigned category of organisms. To enable highly computationally efficient sequence comparisons, in some embodiments, an algorithm may decompose the reads into multiple k-mers (e.g., as described herein). Similarly, for a reference database, known sequences may be pre-processed into sets of indexed k-mers for each organism of interest. However, in some embodiments, the known sequences of the reference sequence database are not pre-processed into sets of indexed k-mers for each organism of interest.

A classification algorithm may rank organisms that are most likely to be present in a given sample based on percent coverage of the references, as well as a score that considers the coverage and uniqueness of the reference sequences that are covered. Furthermore, for each putatively detected organism, a consensus sequence may be assembled from reads to calculate metrics such as percent nucleotide identity. In the case of viruses that tend to have high mutation rates, the comparison with references at the nucleotide level may be enhanced by analysis of translated amino acids at the protein level.

In some embodiments, the reference database comprises a set of polynucleotide reference sequences. In some embodiments, the set of reference polynucleotide sequences comprises more than 100, more than 1000, more than 10,000, more than 100,000, more than 1×10⁶, or more than 1×10⁷ reference sequences. In some embodiments, the identity of the originating species of each reference polynucleotide sequence in the set of reference polynucleotide sequences is known. In some embodiments, each reference polynucleotide sequence in the set of reference polynucleotide sequences represents a gene sequence of a gene from a species. In some embodiments, each reference polynucleotide sequence in the set of reference polynucleotide sequences represents at least 10, 15, 20, 25, 30, 35, 40, 45, or 50 contiguous nucleotides of gene sequence of a gene from a species. In some embodiments, the set of reference polynucleotide sequences includes reference polynucleotide sequences from 10 or more, 100 or more, 1000 or more, 10,000 or more, or 100,000 different species.

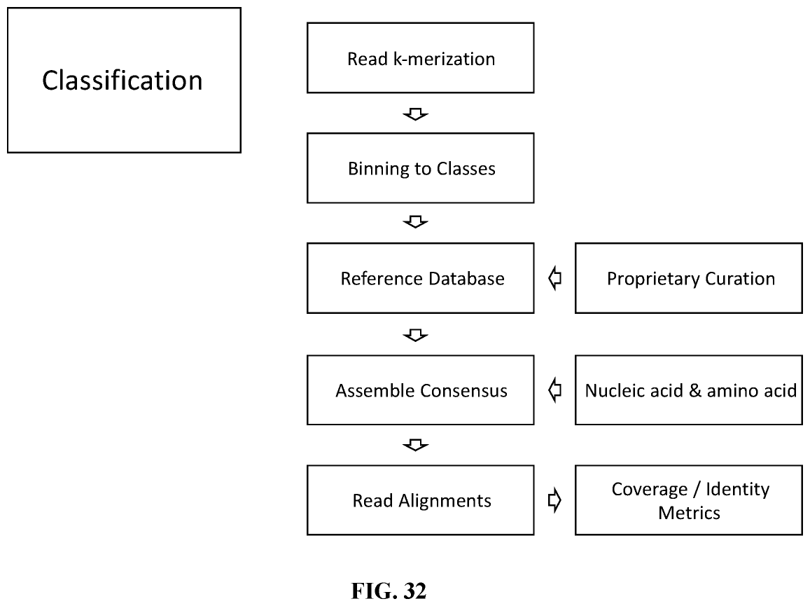

An example classification module is schematically illustrated in FIG. 32.

### Read K-Merization.

A sequencing process may generate a plurality of sequencing reads. As used herein, a “sequencing read” or “sequence read” (also referred to as a “read” or “query sequence”) generally refers to the inferred sequence of nucleotide bases in a nucleic acid molecule. A sequencing read may be an inferred sequence of nucleic acid bases (e.g., nucleotides) or base pairs obtained via a nucleic acid sequencing assay. A sequencing read may be generated using, e.g., next-generation sequencing by a nucleic acid sequencer, such as a massively parallel array sequencer (e.g., Illumina or Pacific Biosciences of California). A sequencing read may correspond to a portion, or in some cases all, of a genome of a subject or species. A sequencing read may be part of a collection of sequencing reads, which may be combined through, for example, alignment (e.g., to a reference genome), to yield a sequence of a genome of a subject. A sequencing read may be of any appropriate length, such as about or more than about 20 nucleotides (nt), 30 nt, 36 nt, 40 nt, 50 nt, 75 nt, 100 nt, 150 nt, 200 nt, 250 nt, 300 nt, 400 nt, 500 nt, or more in length. A sequencing read may be less than 200 nt, 150 nt, 100 nt, 75 nt, or fewer in length. Similarly, a sequencing read for a polypeptide may be of any appropriate length of amino acids, such as about or more than about 20 amino acids (aa), 30 aa, 36 aa, 40 aa, 50 aa, 75 aa, 100 aa, 150 aa, 200 aa, 250 aa, 300 aa, 400 aa, 500 aa, or more in length. A sequencing read may be less than 200 aa, 150 aa, 100 aa, 75 aa, or fewer in length. In some cases, a first sequencing method may be used to provide sequencing reads of a first range of lengths and a second sequencing method may be used to provide sequencing reads of a second range of lengths, where the first range of lengths is longer than the second range of lengths. Sequencing reads may correspond to overlapping sequences of a genome of a subject or may be non-overlapping. Sequencing reads may include functional sequences including adapter and barcode sequences. The functional sequences included in sequencing reads may vary based on nucleic acid processing performed prior to sequencing (e.g., nucleic acid amplification). Sequencing reads may correspond to DNA and/or RNA molecules. Sequencing reads may be “paired,” meaning that they are derived from different ends of a nucleic acid fragment. Paired reads may have intervening unknown sequence or overlap. In some cases, the sequencing read may be a contig or consensus sequence assembled from separate overlapping reads.

A sequencing read may be analyzed in terms of component k-mers. As used herein, “k-mer” generally refers to the subsequences of a given length k that make up a sequencing read. For example, the sequence “AGCTCT” can be divided into the 3-nt subsequences “AGC,” “GCT,” “CTC,” and “TCT.” In this example, each of these subsequences is a k-mer, where k=3. K-mers may be overlapping or non-overlapping. In the above example, “AGC,” “GCT,” “CTC,” and “TCT” are overlapping k-mers. K-mers for the sequences may alternatively be presented as non-overlapping k-mers (e.g., “AGC” and “TCT” only).

A k-mer may be about 3 nucleotides (nt), 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 75 nt, 100 nt, or longer in length. A k-mer may be at least about 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 75 nt, 100 nt, or longer in length. A k-mer may be less than about 30 nt, 25 nt, 20 nt, 15 nt, 10 nt, or shorter in length. A k-mer may be about 3 nt to 10 nt, 3 nt to 13 nt, 3 nt to 15 nt, 3 nt to 20 nt, 3 nt to 25 nt, 3 nt to 30 nt, 3 nt to 35 nt, 3 nt to 40 nt, 3 nt to 45 nt, 3 nt to 50 nt, 3 nt to 55 nt, 3 nt to 60 nt, 3 nt to 65 nt, 3 nt to 70 nt, 3 nt to 75 nt, 3 nt to 80 nt, 3 nt to 85 nt, 3 nt to 90 nt, 3 nt to 95 nt, 3 nt to 99 nt, 5 nt to 10 nt, 5 nt to 15 nt, 5 nt to 15 nt, 5 nt to 20 nt, 5 nt to 25 nt, 5 nt to 30 nt, 5 nt to 35 nt, 5 nt to 40 nt, 5 nt to 45 nt, 5 nt to 50 nt, 5 nt to 55 nt, 5 nt to 60 nt, 5 nt to 65 nt, 5 nt to 70 nt, 5 nt to 75 nt, 5 nt to 80 nt, 5 nt to 85 nt, 5 nt to 90 nt, 5 nt to 95 nt, 5 nt to 99 nt, 7 nt to 10 nt, 7 nt to 17 nt, 7 nt to 15 nt, 7 nt to 20 nt, 7 nt to 25 nt, 7 nt to 30 nt, 7 nt to 35 nt, 7 nt to 40 nt, 7 nt to 45 nt, 7 nt to 50 nt, 7 nt to 55 nt, 7 nt to 60 nt, 7 nt to 65 nt, 7 nt to 70 nt, 7 nt to 75 nt, 7 nt to 80 nt, 7 nt to 85 nt, 7 nt to 90 nt, 7 nt to 95 nt, 7 nt to 99 nt, 10 nt to 15 nt, 10 nt to 20 nt, 10 nt to 25 nt, 10 nt to 30 nt, 10 nt to 35 nt, 10 nt to 40 nt, 10 nt to 45 nt, 10 nt to 50 nt, 10 nt to 55 nt, 10 nt to 60 nt, 10 nt to 65 nt, 10 nt to 70 nt, 10 nt to 75 nt, 10 nt to 80 nt, 10 nt to 85 nt, 10 nt to 90 nt, 10 nt to 95 nt, 10 nt to 99 nt, or any other range therein in length. Similarly, a k-mer may be about 3 amino acids (aa), 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 25 aa, 30 aa, 35 aa, 40 aa, 45 aa, 50 aa, 75 aa, 100 aa, or longer in length. A k-mer may be at least about 3 aa, 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 25 aa, 30 aa, 35 aa, 40 aa, 45 aa, 50 aa, 75 aa, 100 aa, or longer in length. A k-mer may be less than about 30 aa, 25 aa, 20 aa, 15 aa, 10 aa, or shorter in length. A k-mer may be about 3 aa to 10 aa, 3 aa to 13 aa, 3 aa to 15 aa, 3 aa to 20 aa, 3 aa to 25 aa, 3 aa to 30 aa, 3 aa to 35 aa, 3 aa to 40 aa, 3 aa to 45 aa, 3 aa to 50 aa, 3 aa to 55 aa, 3 aa to 60 aa, 3 aa to 65 aa, 3 aa to 70 aa, 3 aa to 75 aa, 3 aa to 80 aa, 3 aa to 85 aa, 3 aa to 90 aa, 3 aa to 95 aa, 3 aa to 99 aa, 5 aa to 10 aa, 5 aa to 15 aa, 5 aa to 15 aa, 5 aa to 20 aa, 5 aa to 25 aa, 5 aa to 30 aa, 5 aa to 35 aa, 5 aa to 40 aa, 5 aa to 45 aa, 5 aa to 50 aa, 5 aa to 55 aa, 5 aa to 60 aa, 5 aa to 65 aa, 5 aa to 70 aa, 5 aa to 75 aa, 5 aa to 80 aa, 5 aa to 85 aa, 5 aa to 90 aa, 5 aa to 95 aa, 5 aa to 99 aa, 7 aa to 10 aa, 7 aa to 17 aa, 7 aa to 15 aa, 7 aa to 20 aa, 7 aa to 25 aa, 7 aa to 30 aa, 7 aa to 35 aa, 7 aa to 40 aa, 7 aa to 45 aa, 7 aa to 50 aa, 7 aa to 55 aa, 7 aa to 60 aa, 7 aa to 65 aa, 7 aa to 70 aa, 7 aa to 75 aa, 7 aa to 80 aa, 7 aa to 85 aa, 7 aa to 90 aa, 7 aa to 95 aa, 7 aa to 99 aa, 10 aa to 15 aa, 10 aa to 20 aa, 10 aa to 25 aa, 10 aa to 30 aa, 10 aa to 35 aa, 10 aa to 40 aa, 10 aa to 45 aa, 10 aa to 50 aa, 10 aa to 55 aa, 10 aa to 60 aa, 10 aa to 65 aa, 10 aa to 70 aa, 10 aa to 75 aa, 10 aa to 80 aa, 10 aa to 85 aa, 10 aa to 90 aa, 10 aa to 95 aa, 10 aa to 99 aa, or any other range therein in length. K-mers analyzed in a given analysis process may vary in length. For example, a first process may analyze k-mers of a first length and a second process may analyze k-mers of a second length, where the first length and second length are not the same. The first length may be longer than the second length. Alternatively, the second length may be longer than the first length. Alternatively or in addition, k-mers of one or more different lengths may be analyzed in a given process (e.g., simultaneously). In an example, a first analysis process may compare k-mers in a sequencing read and a reference sequence that are 21 nt in length, whereas a second analysis process may compare k-mers in a sequencing read and a reference sequence that are 7 nt in length. For any given sequence in a comparison step, k-mers analyzed may be overlapping (such as in a sliding window), and may be of same or different lengths. While k-mers are generally referred to herein as nucleic acid sequences, sequence comparison also encompasses comparison of polypeptide sequences, including comparison of k-mers comprising amino acids.

Sequencing information (e.g., sequencing reads) may be provided in any useful format. For example, sequencing reads may be outputted as FASTQ files and/or in FASTA format. Sequencing information may be included in text file represented as ASCII characters.

In some embodiments k-mer analysis between sequence reads and reference sequences is performed and scored as described in U.S. patent application Ser. No. 15/724,476, entitled “Methods and Systems and Multiple Taxonomic Classification,” filed Oct. 4, 2017, which is hereby incorporated by reference.

### Data Storage and Processing.

Data (e.g., data corresponding to sequencing information, such as sequencing information corresponding to a single sample or a collection of samples) may be initially provided on a local device (e.g., data may be locally stored). Alternatively or in addition, data may be uploaded to a cloud- or web-based storage system (e.g., immediately upon collection or subsequent to collection). For example, data may be collected to a local device and a user may elect to upload the data to a cloud- or web-based storage system (e.g., after performing an initial review of the data). Alternatively, a user may select to have data uploaded to a cloud- or web-based storage system as it is collected. Data may also be stored using a mobile device, such as using a flash drive, memory drive, or other hardware device. Multiple copies of data may be stored for any useful period of time (e.g., to provide a data backup).

Data may include identifying information, such as information about a source or subject from which it derives. Alternatively, identifying information may be separated from the data (e.g., the data may be deidentified) and the data may be associated with a code (e.g., as described herein). In an example, data for multiple different samples is collected and/or processed at a same time, and data for each different sample is assigned a code, which code may or may not include identifying information about the sample.

Data may be of any useful size and in any useful format.

Data may undergo one or more processing steps prior to storage. In an example, raw data may be locally stored and may be subjected to at least one processing step to provide pre-processed data. Pre-processed data may be of a smaller data size (e.g., data may be reduced by processing raw data into chunks, kernals, and/or k-mers) and/or in a different format. Pre-processed data may be transferred to mobile, cloud- or web-based storage and/or may be stored locally. The initially collected raw data may be deleted (e.g., to save room on a hardware device), such as after a predefined period of time. Alternatively, the initially collected raw data may be retained for reference.

Data collected from nucleic acid sequencing may be stored and/or processed separately from data collected from protein sequencing. Alternatively, data collected from nucleic acid sequencing may be stored and/or processed together with from data collected from protein sequencing. In an example, data collected from nucleic acid sequencing corresponding to a sample may be combined with data collected from protein sequencing for subsequent processing. These data may be of the same or different formats.

Data collected from nucleic acid sequencing may be processed separately from data collected from protein sequencing. Alternatively, data collected from nucleic acid sequencing may be processed together with from data collected from protein sequencing. Data collected from nucleic acid sequencing of different types of nucleic acid molecules may also be processed differently. For example, data collected from a first type of nucleic acid molecules (e.g., DNA) may be processed differently than data collected from a second type of nucleic acid molecules (e.g., RNA).

Data may undergo local and/or external processing. For example, sequencing information may be collected using a first processor and may be analyzed using a second processor (e.g., after transfer of data from the first processor to a storage site accessible to the second processor). Data may be processed using a device on which it is locally stored. Alternatively or in addition, data may not be downloaded to a device on which it is processed (e.g., it may be stored in a cloud- or web-based storage system and processed locally). Data may be processed using any useful computing device (e.g., as described herein), including a supercomputing device.

Data may initially be provided in a first file format and changed to a second file format different from the first file format. Transformation to a second file format may append information to the data, such as sample identifying information and/or information about the collection of the data.

### Data Binning.

Data processing may comprise binning sequence information into groups. Groups may include, for example, human, bacterial, fungal, viral/phage, ambiguous, unknown, and other groups. Binning may be based upon comparison of sequences against sequences included in one or more reference databases. Databases against which collected sequences may be compared may be selected by a user (e.g., using a data analysis software interface, such as a web-based software interface). For example, a user may elect to compare collected sequences against a database including reference sequences associated with various bacteria including a bacteria suspected of being included within the sample. Similarly, a user may elect to compare collected sequences against a database including reference sequences associated with the human genome if human DNA is suspected of being included within the sample (e.g., if the source of the sample is a human subject). An analysis program may include a standard set of databases against which sequences may be compared. The program may be configured to allow a user to deselect various databases or include additional databases for analysis.

Binning collected sequences into initial groups may comprise comparing sequences to one or more databases for exact sequence matches (e.g., 100% sequence identity) and/or may provide for some mismatches between collected and stored sequences. A threshold for mismatches (e.g., percent sequence identity required to suggest a match between sequences) may be preloaded into an analysis program and may optionally be altered by a user. Alternatively or in addition, k-mer matching may be used to bin sequences into initial groupings. K-mer matching may be performed for different length k-mers, such as for two or more different length k-mers.

Following binning of collected sequences into initial groups, a sub-binning process may be performed. Sub-binning may be based on exact k-mer matching (e.g., of k-mers of a single size or of multiple different sizes) and/or sequence matching. Sub-binning may also comprise probabilistic analysis such as k-mer weight analysis (e.g., as described herein). Sub-binning for protein sequence analysis may also comprise a multi-frame (e.g., 6-frame) translation process and/or reduced amino acid alphabet analysis.

User input may be provided between each processing step described herein. In some cases, user input may be required for completion of a processing step and commencement of a subsequent processing step. Alternatively, a data analysis workflow may be automated. In an example, user input is requested and provided prior to commencement of a data analysis workflow and user input is not provided between processing steps.

The software routines used to generate the sequence record database and to compare sequencing reads to the database may be run on a computer. The comparison may be performed automatically upon receiving data. The comparison may be performed in response to a user request. The user request may specify which reference database to compare the sample to. The computer may comprise one or more processors. Processors may be associated with one or more controllers, calculation units, and/or other units of a computer system, or implanted in firmware as desired. If implemented in software, the routines may be stored in any computer readable memory, such as in RAM, ROM, flash memory, a magnetic disk, a laser disk, or other storage medium. The record database, sequencing reads, or a report summarizing the results of database construction or sequence read comparison may also be stored in any suitable medium, such as in RAM, ROM, flash memory, a magnetic disk, a laser disk, or other storage medium. Likewise, the record database, sequencing reads, or a report summarizing the results of database construction or sequence read comparison may be delivered to a computing device via any known delivery method including, for example, over a communication channel such as a telephone line, the internet, a wireless connection, etc., or via a transportable medium, such as a computer readable disk, flash drive, etc. A database, sequencing reads, or report may be communicated to a user at a local or remote location using any suitable communication medium. For example, the communication medium may be a network connection, a wireless connection, or an internet connection. A database or report may be transmitted over such networks or connections (or any other suitable means for transmitting information, including but not limited to mailing database summary, such as a print-out) for reception and/or for review by a user. The recipient may be but is not limited to the customer, an individual, a health care provider, a health care manager, or electronic system (e.g. one or more computers, and/or one or more servers). In some cases, the database or report generator sends the report to a recipient's device, such as a personal computer, phone, tablet, or other device. The database or report may be viewed online, saved on the recipient's device, or printed. The comparison of communicated sequencing reads to a database may occur after all the reads are uploaded. The comparison of communicated sequencing reads to a database may begin while the sequencing reads are in the process of being uploaded.

Results of methods described herein may be assembled in a record database.

A record database may comprise reference sequences identified as present in the sample and exclude reference sequences to which no sequencing read was found to correspond, such as by failure to match a sequencing read above a set threshold level. A record database may comprise reference amino acid sequences identified as present in the sample and excludes reference amino acid sequences to which no sequencing read was found to correspond, such as by failure to match a sequencing read above a set threshold level.

The data processing methods and systems provided herein may be used to identify one or more microorganisms and/or viruses and/or parasite and/or antimicrobial resistance markers and/or host response markers within a sample or plurality of samples, where a host can be human or animal or plant. Sources of nucleic acid and protein sequences within a sample or plurality of samples may be identified with individual species (e.g., taxa). The terms “taxon” (plural “taxa”), “taxonomic group,” and “taxonomic unit” are used interchangeably herein to refer to a group of one or more organisms that comprises a node in a clustering tree. The level of a cluster may be determined by its hierarchical order. A taxon may be a group tentatively assumed to be a valid taxon for purposes of phylogenetic analysis. A taxon may be given a name and a rank. For example, a taxon can represent a domain, a sub-domain, a kingdom, a sub-kingdom, a phylum, a sub-phylum, a class, a sub-class, an order, a sub-order, a family, a subfamily, a genus, a subgenus, or a species. Taxa may represent one or more organisms from the kingdoms eubacteria, protista, or fungi at any level of a hierarchal order. A taxon may be a taxonomic unit that is subject in a given analysis (e.g., any of the extant taxonomic units under a given study). A taxon may be known or suspected to be included in a sample under analysis. Alternatively, a taxon may not be known or suspected to be included in a sample under analysis.

The terms “determining”, “measuring”, “evaluating”, “assessing,” “assaying,” and “analyzing” may be used interchangeably herein to refer to any form of measurement, and include determining if an element is present or not (for example, detection). These terms can include both quantitative and/or qualitative determinations. Assessing may be relative or absolute. “Detecting the presence of” can include determining the amount of something present, as well as determining whether it is present or absent.

The term “specificity,” or “true negative rate,” as used herein, generally refers to the ability of a test to exclude a condition correctly. For example, in a classification algorithm, the specificity of the algorithm may refer to the proportion of reads known not to be from an organism in a given taxonomic bin, which may not be placed in the taxonomic bin. In some cases, this is calculated by determining the proportion of true negatives (e.g., reads not placed in the bin that are not from the taxonomic bin) to the total number of reads that are not derived from an organism within the taxonomic bin (e.g., the sum of (i) reads that are not placed in a given taxonomic bin and are not derived from an organism within that taxonomic bin and (ii) reads that are placed in that taxonomic bin that are not derived from an organism within that taxonomic bin).

The term “sensitivity,” or “true positive rate,” as used herein, generally refers to a test's ability to identify a condition correctly. For example, in a classification algorithm, the sensitivity of a test may refer to the proportion of reads known to be from an organism in a given taxonomic bin, which may be placed in the taxonomic bin. In some cases, this is calculated by determining the proportion of true positives (e.g., reads placed in the bin that are from the taxonomic bin) to the total number of reads that are derived from an organism within the taxonomic bin (e.g., the sum of (i) reads that are placed in a given taxonomic bin and are derived from an organism within that taxonomic bin and (ii) reads that are not placed in that taxonomic bin that are derived from an organism within that taxonomic bin).

The quantitative relationship between sensitivity and specificity can change as different classification cut-offs are chosen. This variation can be represented using receiver operating characteristic (ROC) curves. The x-axis of a ROC curve shows the false-positive rate of an assay, which can be calculated as (1−specificity). The y-axis of a ROC curve reports the sensitivity for an assay. This allows one to determine a sensitivity of an assay for a given specificity, and vice versa.

In an aspect, the disclosure provides a method of identifying a plurality of polynucleotides in a sample source. In some cases, the method comprises providing sequencing reads for a plurality of polynucleotides from the sample, and for each sequencing read: (a) performing with a computer system a sequence comparison between the sequencing read and a plurality of reference polynucleotide sequences, where the comparison comprises calculating k-mer weights as a measure of how likely it is that k-mers within the sequencing read are derived from a reference sequence within the plurality of reference polynucleotide sequences; (b) identifying the sequencing read as corresponding to a particular reference sequence in a database of reference sequences if the sum of k-mer weights for the reference sequence is above a threshold level; and (c) assembling a record database comprising reference sequences identified in step (b), where the record database excludes reference sequences to which no sequencing read corresponds.

In another aspect, the disclosure provides a method of identifying one or more taxa in a sample from a sample source. In some cases, the method comprises (a) providing sequencing reads for a plurality of polynucleotides from the sample, and for each sequencing read: (i) performing with a computer system a sequence comparison between the sequencing read and a plurality of reference polynucleotide sequences, where the comparison comprises calculating k-mer weights as a measure of how likely it is that k-mers within the sequencing read are derived from a reference sequence within the plurality of reference polynucleotide sequences; and (ii) calculating a probability that the sequencing read corresponds to a particular reference sequence in a database of reference sequences based on the k-mer weights, thereby generating a sequence probability; (b) calculating a score for the presence or absence of one or more taxa based on the sequence probabilities corresponding to sequences representative of said one or more taxa; and (c) identifying the one or more taxa as present or absent in the sample based on the corresponding scores; or (d) identifying the one or more taxa as present or absent in the sample based on machine learning methods. In some cases, the one or more taxa comprises a first bacterial strain identified as present and a second bacterial strain identified as absent based on one or more nucleotide differences in sequence. In some cases, the first bacterial strain is identified as present and the second bacterial strain is identified as absent based on a single nucleotide difference in sequence.

### Reference Databases.

Analysis of a sequence (e.g., a sequence corresponding to or derived a sample, as described herein) may comprise one or more processes (e.g., comparison processes) in which one or more k-mers of a sequencing read are compared to k-mers of one or more reference sequences (also referred to simply as a “reference”). A reference sequence includes any sequence to which a sequencing read is compared. Typically, the reference sequence is associated with some known characteristic, such as a condition of a sample source, a taxonomic group, a particular species, an expression profile, a particular gene, a particular antimicrobial resistance gene, a particular antiviral resistance gene, a particular antivirulent resistance gene, a particular antiparasitic resistant gene, a particular antiprotozoal resistance gene, an associated phenotype such as likely disease progression, drug resistance or pathogenicity, increased or reduced predisposition to disease, or other characteristic. Typically, a reference sequence is one of many such reference sequences in a database. A variety of databases comprising various types of reference sequences are available, one or more of which may serve as a reference database either individually or in various combinations. A database may comprise many species and sequence types. A database may be a publicly available database. A database may be a specific, locally stored database, such as a database associated with a given sample source. For example, a specific database may provide a comparison between samples collected from a given source over time, such as samples taken from a same subject or location. Examples of databases include, but are not limited to, NR, UniProt, SwissProt. TrEMBL, and UniRef90) databases. A database may comprise specific kinds of sequences from multiple species, such as those used for taxonomic classification of species, such as bacteria. A database may be a 16S database, such as The Greengenes database, the UNITE database, or the SILVA database. Marker genes other than 16S may be used as reference sequences for the identification of microorganisms (e.g. bacteria), such as metabolic genes, genes encoding structural proteins, proteins that control growth, cell cycle or reproductive regulation, housekeeping genes or genes that encode virulence, toxins, or other pathogenic factors. Specific examples of marker genes include, but are not limited to, 18S rDNA, 23 S rDNA, gyrA, gyrB gene, groEL, rpoB gene, fusA gene, recA gene, sod A, coxl gene, and nifD gene. Reference databases can comprise internal transcribed sequences (ITS) databases, such as UNITE, ITSoneDB, or ITS2. A database may comprise multiple sequences from a single species, such as the human genome, the human transcriptome, model organisms such as the mouse genome, the yeast transcriptome, or the C. elegans proteome, or disease vectors such as bat, tick, or mosquitoes and other domestic and wild animals. A reference database may comprise sequences of human transcripts. Reference sequences in databases can comprise DNA sequences, RNA sequences, or protein sequences. Reference sequences in databases can comprise sequences from a plurality of taxa. In some cases, reference sequences may be from a reference individual or a reference sample source. Examples of reference individual genomes include, for example, a maternal genome, a paternal genome, or the genome of a non-cancerous tissue sample. Examples of reference individuals or sample sources include the human genome, the mouse genome, or the genomes of particular serovars, genovars, strains, variants or otherwise characterized types of bacteria, archea, viruses, phages, fungi, and parasites. A database may comprise polymorphic reference sequences that contain one or more mutations with respect to known polynucleotide sequences. Such polymorphic reference sequences may comprise different alleles found in the population, such as single nucleotide polymorphisms (SNPs), indels, microdeletions, microexpansions, common rearrangements, genetic recombinations, or prophage insertion sites, and may contain information on their relative abundance compared to non-polymorphic sequences. Polymorphic reference sequences may also be artificially generated from the reference sequences of a database, such as by varying one or more (including all) positions in a reference genome such that a plurality of possible mutations not in the actual reference database are represented for comparison. A database of reference sequences may comprise reference sequences of one or more of a variety of different taxonomic groups, including, but not limited to, bacteria, archaea, chromalveolata, viruses, fungi, plants, fish, amphibians, reptiles, birds, mammals, and humans. In some cases, a database of reference sequences may consist of sequences from one or more reference individuals or a reference sample sources (e.g. 10, 100, 1000, 10000, 100000, 1000000, or more), and each reference sequence in the database may be associated with its corresponding individual or sample source. An unknown sample may be identified as originating from an individual or sample source represented in a reference database on the basis of a sequence comparison. The databases of reference sequences can comprise reference sequences of one or more genes. The databases of reference sequences can comprise reference sequences of one or more antimicrobial resistant genes, antivirulent resistant genes, antiprotozoal resistant genes, antiviral resistant genes, antiparasitic resistant genes, and/or antifungal resistant genes, etc.

A reference database can consist of sequences (and optionally abundance levels of sequences) associated with one or more conditions. Multiple conditions may be represented by one or more sequences in the reference database, such as 10, 50, 100, 1000, 10000, 100000, 1000000, or more conditions. For example, a reference database may consist of thousands of groups of sequences, each group of sequences being associated with a different bacterial contaminant, such that contamination of a sample by any of the represented bacteria may be detected by sequence comparison according to a method of the disclosure. A condition can be any characteristic of a sample or source from which a sample is derived. For example, the reference database may consist of a set of genes that are associated with contamination by microorganisms, infection of a subject from which the sample is derived, or a host response to pathogens. In some cases, the reference database may consist of a set of antimicrobial genes that are associated with contamination by microorganisms, infection of a subject from which the sample is derived, or a host response to pathogens. Other conditions include, but are not limited to, contamination (e.g., environmental contamination, surface contamination, food contamination, air contamination, water contamination, cell culture contamination), stimulus response (e.g., drug responder or non-responder, allergic response, treatment response), infection (e.g., bacterial infection, fungal infection, viral infection), disease state (e.g., presence of disease, worsening of disease, disease recovery), and a healthy state. In some cases, the reference database may consist of one or more genes associated with antimicrobial resistance, antiviral resistance, antifungal resistance, antibiotic resistance, or antiparasitic resistance, etc. In some cases, the reference database may consist of polynucleotides, amino acid sequences, and/or sequence reads associated with antimicrobial resistant genes, antiviral resistant genes, antifungal resistant genes, or antiparasitic resistant genes, etc. In some cases, the reference database may consist of gene name(s) that confer characteristics (e.g. antimicrobial resistance, antiviral resistance, antivirulent resistance, antifungal resistance, antiprotozoal resistance, antiparasitic resistance, etc.), relevant antibiotics, associated organism(s), resistance mechanism, evidence, metagenomic data, metadata, k-mers, polynucleotides, nucleic acids, protein amino acid sequences, nucleotide sequences, etc. In some cases, the reference database may have metadata. In some cases, metadata may be data information that may provide information about other data. In some cases, metadata may be descriptive metadata, structural metadata, administrative metadata, reference metadata, statistical metadata, etc.

In some cases, the reference database associated with one or more genes may be a publicly available database or a private database. The database may be, for example, MEGARes, Comprehensive Antibiotic Resistance Database (CARD), National Database of Antibiotic Resistant Organisms (NDARO), Structured ARG-database, Antibiotic Resistance Genes Database (ARDB), or RESQU database, etc. The reference database may be populated with data. The data may be, for example, sequence reads, polynucleotides, k-mers, nucleic acids, amino acid sequences, genes (e.g. antimicrobial resistant genes, antiviral resistant genes, antivirulent resistant genes, antifungal resistant genes, antiparasitic resistant genes, antiprotozoal resistant genes, antiprotozoal resistant genes), etc.

Alternatively or additionally, a reference database may be compiled via curation of one or more other databases (including, e.g., one or more publicly available or private databases) and/or evaluation of various controls. Curation of a reference database may comprise assigning probabilistic weights to sequences or portions thereof including k-mers; selection of sequences associated with particular entities or types of entities; enrichment or deletion or sequences associated with particular entities or types of entities; combination of sequence information from one or more different databases, including locally generated databases; analysis of common genetic mutations; etc.

Where the reference database consists of sequences associated with infectious disease or contamination, the sequences may be derived from and associated with any of a variety of infectious agents. The infectious agent can be bacterial. Non-limiting examples of bacterial pathogens include Mycobacteria (e.g. M. tuberculosis, M. bovis, M. avium, M. leprae, and M. africanum), rickettsia, mycoplasma, chlamydia, and legionella. Other examples of bacterial infections include, but are not limited to, infections caused by Gram positive bacillus (e.g., Listeria, Bacillus such as Bacillus anthracis, Erysipelothrix species), Gram negative bacillus (e.g., Bartonella, Brucella, Campylobacter, Enterobacter, Escherichia, Francisella, Hemophilus, Klebsiella, Morganella, Proteus, Providencia, Pseudomonas, Salmonella, Serratia, Shigella, Vibrio and Yersinia species), spirochete bacteria (e.g., Borrelia species including Borrelia burgdorferi that causes Lyme disease), anaerobic bacteria (e.g., Actinomyces and Clostridium species), Gram positive and negative coccal bacteria, Enterococcus species, Streptococcus species, Pneumococcus species, Staphylococcus species, and Neisseria species. Specific examples of infectious bacteria include, but are not limited to: Helicobacter pyloris, Legionella pneumophilia, Mycobacteria tuberculosis, M. avium, M. intracellular e, M. kansaii, M. gordonae, Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus viridans, Streptococcus faecalis, Streptococcus bovis, Streptococcus pneumoniae, Haemophilus influenzae, Bacillus antracis, Erysipelothrix rhusiopathiae, Clostridium tetani, Enterobacter aerogenes, Klebsiella pneumoniae, Pasteurella multocida, Fusobacterium nucleatum, Streptobacillus moniliformis, Treponema pallidium, Treponema pertenue, Leptospira, Rickettsia, and Actinomyces israelii, Acinetobacter, Bacillus, Bordetella, Borrelia, Brucella, Campylobacter, Chlamydia, Chlamydophila, Clostridium, Corynebacterium, Enterococcus, Haemophilus, Helicobacter, Mycobacterium, Mycoplasma, Stenotrophomonas, Treponema, Vibrio, Yersinia, Acinetobacter baumanii, Bordetella pertussis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Corynebacterium diphtheriae, Enterobacter sazakii, Enterobacter agglomerans, Enterobacter cloacae, Enterococcus faecalis, Enterococcus faecium, Escherichia coli, Francisella tularensis, Helicobacter pylori, Legionella pneumophila, Leptospira interrogans, Mycobacterium leprae, Mycobacterium tuberculosis, Mycobacterium ulcerans, Mycoplasma pneumoniae, Pseudomonas aeruginosa, Rickettsia rickettsii, Salmonella typhi, Salmonella typhimurium, Salmonella enterica, Shigella sonnei, Staphylococcus epidermidis, Staphylococcus saprophyticus, Stenotrophomonas maltophilia, Vibrio cholerae, Yersinia pestis, and the like.

Sequences in the reference database may be associated with viral infectious agents. Non-limiting examples of viral pathogens include the herpes virus {e.g., human cytomegalomous virus (HCMV), herpes simplex virus 1 (HSV-1), herpes simplex virus 2 (HSV-2), varicella zoster virus (VZV), Epstein-Barr virus), influenza A virus and Heptatitis C virus (HCV) (see Munger et al, Nature Biotechnology (2008) 26: 1179-1186; Syed et al, Trends in Endocrinology and Metabolism (2009) 21:33-40; Sakamoto et al, Nature Chemical Biology (2005) 1:333-337; Yang et al, Hepatology (2008) 48: 1396-1403) or a picomavirus such as Coxsackievirus B3 (CVB3) (see Rassmann et al, Anti-viral Research (2007) 76: 150-158). Other viruses include, but are not limited to, the hepatitis B virus, HIV, poxvirus, hepadavirus, retrovirus, and RNA viruses such as flavivirus, togavirus, coronavirus, Hepatitis D virus, orthomyxovirus, paramyxovirus, rhabdovirus, bunyavirus, filo virus, Adenovirus, Human herpesvirus, type 8, Human papillomavirus, BK virus, JC virus, Smallpox, Hepatitis B virus, Human bocavirus, Parvovirus B19, Human astrovirus, Norwalk virus, coxsackievirus, hepatitis A virus, poliovirus, rhinovirus, Severe acute respiratory syndrome virus, Hepatitis C virus, yellow fever virus, dengue virus, West Nile virus, Rubella virus, Hepatitis E virus, and Human immunodeficiency virus (HIV). In certain cases, the virus is an enveloped virus. Examples include, but are not limited to, viruses that are members of the hepadnavirus family, herpesvirus family, iridovirus family, poxvirus family, flavivirus family, togavirus family, retrovirus family, coronavirus family, filovirus family, rhabdovirus family, bunyavirus family, orthomyxovirus family, paramyxovirus family, and arenavirus family. Other examples include, but are not limited to, Hepadnavirus hepatitis B virus (HBV), woodchuck hepatitis virus, ground squirrel (Hepadnaviridae) hepatitis virus, duck hepatitis B virus, heron hepatitis B virus, Herpesvirus herpes simplex virus (HSV) types 1 and 2, varicella-zoster virus, cytomegalovirus (CMV), human cytomegalovirus (HCMV), mouse cytomegalovirus (MCMV), guinea pig cytomegalovirus (GPCMV), Epstein-Barr virus (EBV), human herpes virus 6 (HHV variants A and B), human herpes virus 7 (HHV-7), human herpes virus 8 (HHV-8), Kaposi's sarcoma—associated herpes virus (KSHV), B virus Poxvirus vaccinia virus, variola virus, smallpox virus, monkeypox virus, cowpox virus, camelpox virus, ectromelia virus, mousepox virus, rabbitpox viruses, raccoonpox viruses, molluscum contagiosum virus, orf virus, milker's nodes virus, bovin papullar stomatitis virus, sheeppox virus, goatpox virus, lumpy skin disease virus, fowlpox virus, canarypox virus, pigeonpox virus, sparrowpox virus, myxoma virus, hare fibroma virus, rabbit fibroma virus, squirrel fibroma viruses, swinepox virus, tanapox virus, Yabapox virus, Flavivirus dengue virus, hepatitis C virus (HCV), GB hepatitis viruses (GBV-A, GBV-B and GBV-C), West Nile virus, yellow fever virus, St. Louis encephalitis virus, Japanese encephalitis virus, Powassan virus, tick-borne encephalitis virus, Kyasanur Forest disease virus, Togavirus, Venezuelan equine encephalitis (VEE) virus, chikungunya virus, Ross River virus, Mayaro virus, Sindbis virus, rubella virus, Retrovirus human immunodeficiency virus (HIV) types 1 and 2, human T cell leukemia virus (HTLV) types 1, 2, and 5, mouse mammary tumor virus (MMTV), Rous sarcoma virus (RSV), lentiviruses, Coronavirus, severe acute respiratory syndrome (SARS) virus, Filovirus Ebola virus, Marburg virus, Metapneumoviruses (MPV) such as human metapneumovirus (HMPV), Rhabdovirus rabies virus, vesicular stomatitis virus, Bunyavirus, Crimean-Congo hemorrhagic fever virus, Rift Valley fever virus, La Crosse virus, Hantaan virus, Orthomyxovirus, influenza virus (types A, B, and C), Paramyxovirus, parainfluenza virus (PIV types 1, 2 and 3), respiratory syncytial virus (types A and B), measles virus, mumps virus, Arenavirus, lymphocytic choriomeningitis virus, Junin virus, Machupo virus, Guanarito virus, Lassa virus, Ampari virus, Flexal virus, Ippy virus, Mobala virus, Mopeia virus, Latino virus, Parana virus, Pichinde virus, Punta toro virus (PTV), Tacaribe virus and Tamiami virus. In some cases, the virus is a non-enveloped virus, examples of which include, but are not limited to, viruses that are members of the parvovirus family, circovirus family, polyoma virus family, papillomavirus family, adenovirus family, iridovirus family, reovirus family, birnavirus family, calicivirus family, and picornavirus family. Specific examples include, but are not limited to, canine parvovirus, parvovirus B19, porcine circovirus type 1 and 2, BFDV (Beak and Feather Disease virus, chicken anaemia virus, Polyomavirus, simian virus 40 (SV40), JC virus, BK virus, Budgerigar fledgling disease virus, human papillomavirus, bovine papillomavirus (BPV) type 1, cotton tail rabbit papillomavirus, human adenovirus (HAdV-A, HAdV-B, HAdV-C, HAdV-D, HAdV-E, and HAdV-F), fowl adenovirus A, bovine adenovirus D, frog adenovirus, Reovirus, human orbivirus, human coltivirus, mammalian orthoreovirus, bluetongue virus, rotavirus A, rotaviruses (groups B to G), Colorado tick fever virus, aquareovirus A, cypovirus 1, Fiji disease virus, rice dwarf virus, rice ragged stunt virus, idnoreovirus 1, mycoreovirus 1, Birnavirus, bursal disease virus, pancreatic necrosis virus, Calicivirus, swine vesicular exanthema virus, rabbit hemorrhagic disease virus, Norwalk virus, Sapporo virus, Picornavirus, human polioviruses (1-3), human coxsackieviruses Al-22, 24 (CAI-22 and CA24, CA23 (echovirus 9)), human coxsackieviruses (Bl-6 (CB1-6)), human echoviruses 1-7, 9, 11-27, 29-33, vilyuish virus, simian enteroviruses 1-18 (SEV1-18), porcine enteroviruses 1-11 (PEVI-11), bovine enteroviruses 1-2 (BEV1-2), hepatitis A virus, rhinoviruses, hepatoviruses, cardio viruses, aphthoviruses and echoviruses. The virus may be phage. Examples of phages include, but are not limited to T4, T5, λ phage, T7 phage, G4, PI, φ6, Thermoproteus tenax virus 1, M13, MS2, Qβ, φX174, Φ29, PZA, Φ15, BS32, B103, M2Y (M2), Nf, GA-1, FWLBc1, FWLBc2, FWLLm3, B4. The reference database may comprise sequences for phage that are pathogenic, protective, or both. In some cases, the virus is selected from a member of the Flaviviridae family (e.g., a member of the Flavivirus, Pestivirus, and Hepacivirus genera), which includes the hepatitis C virus, Yellow fever virus; Tick-borne viruses, such as the Gadgets Gully virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus and the Negishi virus; seabird tick-borne viruses, such as the Meaban virus, Saumarez Reef virus, and the Tyuleniy virus; mosquito-borne viruses, such as the Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus; and viruses with no known arthropod vector, such as the Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, and the Cell fusing agent virus. In some cases, the virus is selected from a member of the Arenaviridae family, which includes the Ippy virus, Lassa virus (e.g., the Josiah, LP, or GA391 strain), lymphocytic choriomeningitis virus (LCMV), Mobala virus, Mopeia virus, Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Whitewater Arroyo virus, Chapare virus, and Lujo virus. In some cases, the virus is selected from a member of the Bunyaviridae family (e.g., a member of the Hantavirus, Nairovirus, Orthobunyavirus, and Phlebovirus genera), which includes the Hantaan virus, Sin Nombre virus, Dugbe virus, Bunyamwera virus, Rift Valley fever virus, La Crosse virus, Punta Toro virus (PTV), California encephalitis virus, and Crimean-Congo hemorrhagic fever (CCHF) virus. In some cases, the virus is selected from a member of the Filoviridae family, which includes the Ebola virus (e.g., the Zaire, Sudan, Ivory Coast, Reston, and Uganda strains) and the Marburg virus (e.g., the Angola, Ci67, Musoke, Popp, Ravn and Lake Victoria strains); a member of the Togaviridae family (e.g., a member of the Alphavirus genus), which includes the Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O'nyong'nyong virus, and the chikungunya virus; a member of the Poxviridae family (e.g., a member of the Orthopoxvirus genus), which includes the smallpox virus, monkeypox virus, and vaccinia virus; a member of the Herpesviridae family, which includes the herpes simplex virus (HSV; types 1, 2, and 6), human herpes virus (e.g., types 7 and 8), cytomegalovirus (CMV), Epstein-Barr virus (EBV), Varicella-Zoster virus, and Kaposi's sarcoma associated-herpesvirus (KSHV); a member of the Orthomyxoviridae family, which includes the influenza virus (A, B, and C), such as the H5N1 avian influenza virus or H1N1 swine flu; a member of the Coronaviridae family, which includes the severe acute respiratory syndrome (SARS) virus; a member of the Rhabdoviridae family, which includes the rabies virus and vesicular stomatitis virus (VSV); a member of the Paramyxoviridae family, which includes the human respiratory syncytial virus (RSV), Newcastle disease virus, hendravirus, nipahvirus, measles virus, rinderpest virus, canine distemper virus, Sendai virus, human parainfluenza virus (e.g., 1, 2, 3, and 4), rhinovirus, and mumps virus; a member of the Picornaviridae family, which includes the poliovirus, human enterovirus (A, B, C, and D), hepatitis A virus, and the coxsackievirus; a member of the Hepadnaviridae family, which includes the hepatitis B virus; a member of the Papillamoviridae family, which includes the human papilloma virus; a member of the Parvoviridae family, which includes the adeno-associated virus; a member of the Astroviridae family, which includes the astrovirus; a member of the Polyomaviridae family, which includes the JC virus, BK virus, and SV40 virus; a member of the Calciviridae family, which includes the Norwalk virus; a member of the Reoviridae family, which includes the rotavirus; and a member of the Retroviridae family, which includes the human immunodeficiency virus (HIV; e.g., types 1 and 2), and human T-lymphotropic virus Types I and II (HTLV-1 and HTLV-2, respectively).

Antivirulent resistant genes may be associated with a virulent strain as described elsewhere herein. In some cases, antivirulent resistant genes may be unique for a particular virulent strain, or shared by several virulent strains. Examples of virulence genes include, but are not limited to, various toxin and pathogenicity factor genes, such as those encoding immunoglobulin-binding proteins, serum opacity factor, M protein, C5a peptidase, Fc-binding proteins, collagenase, hyaluronate lyase, streptococcal pyrogenic exotoxins, mitogenic factor, alpha C protein, fibrinogen binding protein, fibronectin binding protein, coagulase, enterotoxins, exotoxins, leukocidins, or V8 protease. In some cases, genes which confer resistance to virulence may be present on plasmids in a cell.

Infectious agents with which sequences in a reference database may be associated can be fungal. Examples of infectious fungal agents include, without limitation Aspergillus, Blastomyces, Coccidioides, Cryptococcus, Histoplasma, Paracoccidioides, Sporothrix, and at least three genera of Zygomycetes. Fungal agents may be associated with various diseases and conditions in humans, companion animals, and other species. For example, fungal agents may be associated with rashes including diaper rash. Examples of organisms that cause disease in animals include Malassezia furfur, Epidermophyton floccosur, Trichophyton mentagrophytes, Trichophyton rubrum, Trichophyton tonsurans, Trichophyton equinum, Dermatophilus congolensis, Microsporum canis, Microsporu audouinii, Microsporum gypseum, Malassezia ovale, Pseudallescheria, Scopulariopsis, Scedosporium, and Candida albicans. Further examples of fungal infectious agents include, but are not limited to, Aspergillus, Blastomyces dermatitidis, Candida, Coccidioides immitis, Cryptococcus neoformans, Histoplasma capsulatum var. capsulatum, Paracoccidioides brasiliensis, Sporothrix schenckii, Zygomycetes spp., Absidia corymbifera, Rhizomucor pusillus, and Rhizopus arrhizus.

Another example of infectious agents with which sequences in a reference database may be associated are parasites. Non-limiting examples of parasites include Plasmodium, Leishmania, Babesia, Treponema, Borrelia, Trypanosoma, Toxoplasma gondii, Plasmodium falciparum, P. vivax, P. ovale, P. malariae, Trypanosoma spp., or Legionella spp.

A reference database may combine sequences associated with different infectious agents (e.g., reference sequences associated with infection by a variety of bacterial agents, a variety of viral agents, and a variety of fungal agents). Moreover, a reference database may comprise sequences identified as originating from a pathogen that has not yet been identified or classified.

Reference sequences associated with a condition also include genetic markers for drug resistance, pathogenicity, and disease. A variety of disease-associated markers are known, which may be represented in the reference database. A disease-associated marker may be a causal genetic variant. In general, causal genetic variants are genetic variants for which there is statistical, biological, and/or functional evidence of association with a disease or trait. A single causal genetic variant can be associated with more than one disease or trait. In some cases, a causal genetic variant can be associated with a Mendelian trait, a non-Mendelian trait, or both. Causal genetic variants can manifest as variations in a polynucleotide, such 1, 2, 3, 4, 5, 6, 7, 8, 9, 10, 20, 50, or more sequence differences (such as between a polynucleotide comprising the causal genetic variant and a polynucleotide lacking the causal genetic variant at the same relative genomic position). Non-limiting examples of types of causal genetic variants include single nucleotide polymorphisms (SNP), deletion/insertion polymorphisms (DIP), copy number variants (CNV), short tandem repeats (STR), restriction fragment length polymorphisms (RFLP), simple sequence repeats (SSR), variable number of tandem repeats (VNTR), randomly amplified polymorphic DNA (RAPD), amplified fragment length polymorphisms (AFLP), mter-retrotransposon amplified polymorphisms (IRAP), long and short interspersed elements (LINE/SINE), long tandem repeats (LTR), mobile elements, retrotransposon microsatellite amplified polymorphisms, retrotransposon-based insertion polymorphisms, sequence specific amplified polymorphism, and heritable epi genetic modification (for example, DNA methylation). A causal genetic variant may also be a set of closely related causal genetic variants. Some causal genetic variants may exert influence as sequence variations in RNA polynucleotides. At this level, some causal genetic variants are also indicated by the presence or absence of a species of RNA polynucleotides. Also, some causal genetic variants result in sequence variations in protein polypeptides. There are various causal genetic variants. An example of a causal genetic variant that is a SNP is the Hb S variant of hemoglobin that causes sickle cell anemia. An example of a causal genetic variant that is a DIP is the delta508 mutation of the CFTR gene which causes cystic fibrosis. An example of a causal genetic variant that is a CNV is trisomy 21, which causes Down's syndrome. An example of a causal genetic variant that is an STR is tandem repeat that causes Huntington's disease. Additional non-limiting examples of causal genetic variants are described in WO2014015084A2 and US20100022406. Examples of drug resistance markers include enzymes conferring resistance to various aminoglycoside antibiotics such as G418 and neomycin (e.g., an aminoglycoside 3′-phosphotransferase, 3′APH II, also known as neomycin phosphotransferase II (nptII or “neo”)), Zeocin™ or bleomycin (e.g., the protein encoded by the ble gene from Streptoalloteichus hindustanus), hygromycin (e.g., hygromycin resistance gene, hph, from Streptomyces hygroscopicus or from a plasmid isolated from Escherichia coli or Klebsiella pneumoniae, which codes for a kinase (hygromycin phosphotransferase, HPT) that inactivates Hygromycin B through phosphorylation), puromycin (e.g., the Streptomyces alboniger puromycin-N-acetyl-transferase (pac) gene), or blasticidin (e.g., an acetyl transferase encoded by the bls gene from Streptoverticillum sp. JCM 4673, or a deaminase encoded by a gene such as bsr, from Bacillus cereus or the BSD resistance gene from Aspergillus terreus). Other drug resistance markers include, for example, dihydrofolate reductase (DHFR), adenosine deaminase (ADA), thymidine kinase (TK), and hypoxanthine-guanine phosphoribosyltransferase (HPRT). Proteins such as P-glycoprotein and other multidrug resistance proteins act as pumps through which various cytotoxic compounds, e.g., chemotherapeutic agents such as vinblastine and anthracyclines, are expelled from cells. Markers of pathogenicity include, for example, factors involved in outer-membrane protein expression, microbial toxins, factors involved in biofilm formation, factors involved in carbohydrate transport and metabolism, factors involved in cell envelope synthesis, and factors involved in lipid metabolism. Markers of pathogenicity can include, but are not limited to, for example, gp120, ebola virus envelope protein, or other glycosylated viral envelope proteins or viral proteins.

A reference database may consist of host expression profiles associated with a healthy state and/or one or more disease states, in which certain combinations of expressed genes (or levels of expression of particular genes) identify a condition of a subject. The groups of genes may be overlapping. The reference database consisting of sequences associated with a condition may comprise both host expression profiles and groups of sequences associated with other conditions (e.g. reference sequences associated with various infectious agents).

In another example, a reference database can comprise sequences associated with contamination, such as polynucleotide and/or amino acid sequences from food contaminants, surface contaminants, or environmental contaminants. Examples of common food contaminants are Escherichia coli, Clostridium botulinum, Salmonella, Listeria, and Vibrio cholerae. Examples of surface contaminants are Escherichia coli, Clostridium botulinum, Salmonella, Listeria, Vibrio cholerae, influenza virus, methicillin-resistant Staphylococcus aureus, vancomycin-resistant Enterococci, Pseudomonas spp., Acinetobacter spp., Clostridium difficile, and norovirus. Examples of environmental contaminants are fungi such as Aspergillus and Wallemia sebi; chromalveolata such as dinoflagellates; amoebae; viruses; and bacteria. Contaminants may be infectious agents, examples of which are provided herein.

In some cases, a database of references sequence comprises polynucleotide sequences reverse-translated from amino acid sequences. In this context, translation refers to the process of using the codon code to determine an amino acid sequence from a nucleotide sequence. The standard codon code is degenerate, such that multiple three-nucleotide codons encode the same amino acid. As such, reverse-translation often produces a variety of possible sequences that could encode a particular amino acid sequence. In some cases, to simplify this process, reverse-translation can use a non-degenerate code, such that each amino acid is only represented by a single codon. For example, in the standard DNA codon system, phenylalanine is encoded by “TTT” and “TTC.” A non-degenerate code may only associate one of the codons with phenylalanine. A sequencing read can be compared to this non-degenerate, reverse-translated sequence by any of the methods described herein. Furthermore, the sequencing read can be translated into all six reading-frames and reverse-translated using the same non-degenerate code to generate six polynucleotides that do not include alternate codons prior to comparing. By reverse-translating a reference amino acid sequence, and comparing it to sequencing reads translated then reverse-translated using the same reverse-translation code, nucleic acid sequences may be analyzed in the protein space.

Access to a reference database may be provided via a web-based connection. Alternatively, a reference database may be locally stored, or may be stored in an accessible cloud-, web-, or mobile location.

A reference database may be updated manually and/or by a computer. A reference database may require expert knowledge to manually collect, correct, and/or annotate the classification database data. A reference database may be updated by a crowd sourcing. A reference database may be altered as described elsewhere herein.

### Assembling Sequences.

Assembling sequences from sequencing reads associated with a given sample (e.g., sequencing reads identified via sequencing assays, as described herein, and/or assigned to a given control) may comprise analyzing sequencing reads or portions thereof exact sequence matching, using k-mer analyses, probabilistic analyses, in view of other sequencing reads or portions thereof included in a given sample, in view of knowledge of a given sample's contents and/or origin, comparison to one or more reference databases, etc. Identifying a sequence associated with a given sample or control may comprise exact sequence matching. However, certain sequences are known to be conserved across a plurality of species of a given classification, sometimes with only minor base differences. Accordingly, identifying microorganisms and pathogens within a given sample or control at a species level may require a more rigorous analysis, as described herein. Identifying a sequence associated with a given sample or control may comprise consensus analysis. Identifying a sequence associated with a given sampler or control may comprise identification of one or more genes, including anti-microbial resistance genes.

### K-Mer Analysis.

In addition or as an alternative to exact sequence matching, k-mer analysis may be used to identify sequences as corresponding to various sources, such as various microorganisms and/or viruses. Reference sequences in a given database of reference sequences may be associated with k-mers of given lengths (e.g., prior to comparison with collected sequences). Each reference sequence in a database of reference sequences may be associated with, prior to the comparison, a k-mer weight as a measure of how likely it is that a k-mer within the reference sequence originates from the reference sequence. Alternatively, the database of reference sequences can comprise sequences from a plurality of taxa, and each reference sequence in the database of reference sequences is associated with a k-mer weight as a measure of how likely it is that a k-mer within the reference sequence originates from a taxon within the plurality of taxa. Calculating the k-mer weight can comprise comparing a reference sequence in the database to the other reference sequences in the database, such as by a method described herein. The k-mer values thus associated with sequences or taxa in the database may then be used in determining k-mer weights for k-mers within sequencing reads.

Comparing k-mers in a sequence (e.g., a nucleic acid sequence, such as a sequencing read, or an amino acid sequence) to a reference sequence may comprise counting k-mer matches between the two. The stringency for identifying a match may vary. For example, a match may be an exact match, in which a nucleotide sequence of a k-mer from a sequencing read is identical to a nucleotide sequence of a k-mer from a reference sequence. Alternatively, a match may be an incomplete match, in which 1, 2, 3, 4, 5, 10, or more mismatches between a k-mer of a sequencing read and a k-mer of a reference sequence are permitted. In addition to counting matches, a likelihood (also referred to as a “k-mer weight” or “KW”) can be calculated. A k-mer weight may relate a count of a particular k-mer within a particular reference sequence, a count of the particular k-mer among a group of sequences comprising the reference sequence, and a count of the particular k-mer among all reference sequences in the database of reference sequences. In one embodiment, the k-mer weight is calculated according to the following formula, which calculates the k-mer weight as a measure of how likely it is that a particular k-mer (Kᵢ) originates from a reference sequence (refᵢ) as follows:

$$KWre {f}_{i} = \frac{{C}_{r e f} ( {K}_{i} ) / {C}_{db} ( {K}_{i} )}{{C}_{db} ( {K}_{i} ) / Total kmer count} \qquad ( Eqn . 1 )$$

where C represents a function that returns the count of Kᵢ, C_{ref}(Kᵢ) indicates the count of the Kᵢ in a particular reference sequence, C_{db}(Kᵢ) indicates the count of Kᵢ in the database, and Total kmer count is the total number of kmers in the database. This weight provides a relative, database specific measure of how likely it is that a k-mer originated from a particular reference. However, other measures for weighting a k-mer are possible. For instance, some embodiments, the k-mer weight is calculated according to the following formula, which calculates the k-mer weight as a measure of how likely it is that a particular k-mer (Kᵢ) originates from a reference sequence (refᵢ) as follows:

KWrefᵢ(Kᵢ)=C_{ref}(Kᵢ)/C_{db}(Kᵢ) (Eqn. 2)
where C represents a function that returns the count of Kᵢ, and C_{ref}(Kᵢ) indicates the count of the Kᵢ in a particular reference sequence. In still other embodiments, the k-mer weight is calculated according to the following formula, which calculates the k-mer weight as a measure of how likely it is that a particular k-mer (Kᵢ) originates from a reference sequence (refᵢ) as follows:

$$K W r e {f}_{i} ( {K}_{i} ) = {log}_{x} [ \frac{{C}_{r e f} ( {K}_{i} ) / {C}_{db} ( {K}_{i} )}{{C}_{db} ( {K}_{i} ) / Total kmer count} ] \qquad ( Eqn . 3 )$$

where C represents a function that returns the count of Kᵢ, C_{ref}(Kᵢ) indicates the count of the Kᵢ in a particular reference sequence, C_{db}(Kᵢ) indicates the count of Kᵢ in the database, Total kmer count is the total number of kmers in the database, and x is a base for the logarithm (e.g., 10, π, or any other base). In still other embodiments, the k-mer weight is calculated according to the following formula, which calculates the k-mer weight as a measure of how likely it is that a particular k-mer (Kᵢ) originates from a reference sequence (refᵢ) as follows:

$$K W r e {f}_{i} ( {K}_{i} ) = \frac{{C}_{r e f} ( {K}_{i} ) / {C}_{db} ( {K}_{i} )}{{C}_{db} ( {K}_{i} ) / {log}_{x} [ Total kmer count ]} \qquad ( Eqn . 4 )$$

where C represents a function that returns the count of Kᵢ, C_{ref}(Kᵢ) indicates the count of the Kᵢ in a particular reference sequence, C_{db}(Kᵢ) indicates the count of Kᵢ in the database, Total kmer count is the total number of kmers in the database, and x is a base for the logarithm (e.g., 10, π, or any other base).

Prior to comparing a sequencing read to the database of reference sequences, the k-mer weight (or measurement of likelihood that a k-mer originates from a given reference sequence) can be calculated for each k-mer and reference sequence in the database. In some cases, when a reference databases comprises sequences from a plurality of taxa, each reference sequence can be associated with a measure of likelihood, or k-mer weight, that a k-mer within the reference sequence originates from a taxon within a plurality of taxa. As a non-limiting example, a reference database can comprise sequences from multiple species of canines, and the k-mer weight could be calculated by relating the count of a given k-mer in all canine sequences to its count in the entire database, which includes other taxa. In some examples, the k-mer weight measuring how likely it is that a k-mer originates from a specific taxon is calculated by defining C_{ref}(Kᵢ) in the above equation as a function that returns the total count of Kᵢ in a particular taxon.

For each reference sequence, reference database derived weights for a plurality of k-mers within a sequencing read may be added and compared to a threshold value. The threshold value can be specific to the collection of reference sequences in the database and may be selected based on a variety of factors, such as average read length, whether a specific sequence or source organism is to be identified as present in the sample, and the like. A threshold value may be alterable by a user. If the sum of k-mer weights for the reference sequence is above the threshold level, the sequencing read may be identified as corresponding to the reference sequence, and optionally the organism or taxonomic group associated with the reference sequence. In some cases, the read is assigned to the reference sequence with the maximum sum of k-mer weights, which may or may not be required to be above a threshold. In the case of a tie, where a sequence read has an equal likelihood of belonging to more than one reference sequence as measured by k-mer weight, the sequence read can be assigned to the taxonomic lowest common ancestor (LCA) taking into account the read's total k-mer weight along each branch of the phylogenetic tree. In general, correspondence with a reference sequence, organism, or taxonomic group indicates that it was present in the sample.

In some embodiments, the present disclosure comprises calculating a probability. In some embodiments, a probability is calculated for a sequencing read generated from a plurality of polynucleotides. In some embodiments, the probability is the probability (or likelihood) that the sequencing read corresponds to a particular reference sequence in a database of reference sequences based on the k-mer weights. A probability may be calculated for each sequencing read, thereby generating a plurality of sequence probabilities. In some cases, the presence or absence of one or more taxa in a sample may be determined based on the sequence probabilities. For example, the probability may identify a first bacterial strain as being present in the sample and a second bacterial strain as being absent in the sample. In some cases, the probability is represented as a percentage (%) or as a fraction. In some cases, the presence or absence of one or more genes in a sample may be determined based on the sequence probabilities. For example, the probability may identify a first gene as being present in the sample and a second gene as being absent in the sample. In some embodiments, the probability is represented as a percentage (%) or as a fraction. In some embodiments, a probability is provided as a score representative of the probability. The score can be based on any arbitrary scale so long as the score is indicative of the probability (e.g. a probability that an individual sequence corresponds to a particular reference sequence, a probability that a particular taxon is present in the sample, or a probability that an individual sequence corresponds to a particular reference sequence). The probability or a score representative of the probability may be used to determine the presence or absence of one or more taxa within a sample. For example, a probability or score above a threshold value may be indicative of presence, and/or a probability or score below a threshold value may be indicative of absence. The probability or a score representative of the probability may be used to determine the presence or absence of one or more genes (e.g. one or more antimicrobial resistance gene, antiprotozoal resistance gene, antiviral resistance gene, antivirulent resistance gene, antifungal resistance gene, antiparasitic gene, etc.) within a sample. The probability or a score representative of the probability may be used to determine the presence or absence of one or more genes within a sample. In some embodiments, presence or absence is reported as a probability, rather than an absolute call. Example methods for calculating such probabilities are provided herein. In general, examples described herein in terms of presence or absence likewise encompass calculating a probability or score for such presence or absence.

### Sequence Identification.

One or more steps of a method described herein may be performed in parallel for each of a plurality of sequencing reads (e.g., a plurality of sequencing reads generated from a nucleic acid sequencing process). For example, each of the sequencing reads in a plurality of sequencing reads may be subjected in parallel to a first sequence comparison between the sequencing read and a plurality of reference polynucleotide sequences (e.g. reference polynucleotide sequences from a plurality of different taxa and/or a plurality of different reference databases). Comparison in parallel may differ from certain stepwise comparison processes in that sequencing reads having a purported match in a first reference database may not be subtracted from the query set of sequences for subsequent comparison with a second reference database. In such a stepwise process, sequences having a purported match in the first database may be incorrectly identified before comparison being run against a reference database containing a more accurate match (e.g., the correct sequence). Instead, by running a comparison against a plurality of different reference sequences corresponding to a plurality of different taxa, each sequence can be assigned to an optimal first taxonomic class prior to identifying with greater specificity a sequence or taxon to which a sequencing read corresponds. For example, sequencing reads may be first classified as corresponding to human, bacterial, or fungal sequences before identifying a particular gene, bacterial species, or fungal species to which the sequencing read corresponds. In some instances, this process is referred to as “binning.” Parallel sequence comparison may comprise comparison with sequences from two or more different taxonomic groups, such as 3, 4, 5, 6, or more different taxonomic groups. In some cases, the different taxonomic groups may be selected from two or more of the following bacteria, archaea, viruses, fungi, plants, fish, amphibians, reptiles, birds, mammals, and humans.

Identifying components within a sample may further comprise quantifying an amount of polynucleotides corresponding to a reference sequence identified in an earlier step. The accuracy of a quantification method may depend on the sequencing methods and/or preprocessing methods used to analyze a sample, as well as details of sample collection, storage, and preparation (e.g., as described herein). A quantification method may analyze absolute or relative quantities of components within a given sample. Quantification can be based on a number of corresponding sequencing reads identified. Quantification can be based on a number of corresponding sequencing reads identified associated with a particular gene (e.g. antimicrobial resistance gene, antiviral resistance gene, antivirulent resistance gene, antiprotozoal resistance genes antifungal resistance gene, antiparasitic resistance gene, etc.). This can include normalizing the count by the total number of reads, the total number of reads associated with sequences, the length of the reference sequence, or a combination thereof. Examples of such normalization include FPKM and RPKM, but may also include other methods that take into account the relative amount of reads in different samples, such as normalizing sequencing reads from samples by the median of ratios of observed counts per sequence. A difference in quantity between samples can indicate a difference between the two samples. The quantitation can be used to identify differences between subjects, such as comparing the taxa present in the microbiota of subjects with different diets, or to observe changes in the same subject over time, such as observing the taxa present in the microbiota of a subject before and after going on a particular diet. The quantitation can be used to direct remedial treatment for a subject. In some cases, quantitation of an antimicrobial gene may direct the use of antimicrobial medicines or combinatorial therapeutics. In some cases, quantitation may be used to select a treatment which attenuates or eliminates the expression or protein activity of the antimicrobial resistance gene (e.g., by antisense RNA, RNA interference (RNAi) sequences, antibodies, or small molecule inhibitors).

A method may comprise determining the presence, absence, or abundance of specific taxa or nucleotide polymorphisms within samples based on results of an earlier step. The plurality of reference polynucleotide sequences may comprise groups of sequences corresponding to individual taxa in the plurality of taxa. In some cases, at least 50, 100, 250, 500, 1000, 5000, 10000, 50000, 100000, 250000, 500000, or 1000000 different taxa may be identified as absent or present (and optionally abundance, which may be relative) based on sequences analyzed by a method described herein. In some cases, this analysis may be performed in parallel. The methods, compositions, and systems of the present disclosure may enable parallel detection of the presence or absence of a taxon in a community of taxa, such as an environmental or clinical sample, when the taxon identified comprises less than one per 10⁹, or one per 10⁶, or 0.05% of the total population of taxa in the source sample. Detection may be based on sequencing reads corresponding to a polynucleotide that is present at less than 0.01% of the total nucleic acid population. The particular polynucleotide may be at least 20%, 30%, 40%, 50%, 60%, 70%, 80%, 85%, 90%, 91%, 92%, 93%, 94%, 95%, 96% or 97% homologous to other nucleic acids in the population. In some cases, the particular polynucleotide is less than 75%, 50%, 40%, 30%, 20%, or 10% homologous to other nucleic acids in the population. Determining the presence, absence, or abundance of specific taxa can comprise identifying an individual subject as the source of a sample. For example, a reference database may comprise a plurality of reference sequences, each of which corresponds to an individual organism (e.g., a human subject), with sequences from a plurality of different subject represented among the reference sequences. Sequencing reads for an unknown sample may then be compared to sequences of the reference database, and based on identifying the sequencing reads in accordance with a described method, an individual represented in the reference database may be identified as the sample source of the sequencing reads. In such a case, the reference database may comprise sequences from at least 10², 10³, 10⁴, 10⁵, 10⁶, 10⁷, 10⁸, 10⁹, or more individuals.

In some cases, a sequencing read does not have a match to a reference sequence at the level of a particular taxonomic group (e.g. at the species level), or at any taxonomic level. When no match is found, the corresponding sequence may be added to a reference database on the basis of known characteristics. In some cases, when a sequence is identified as belonging to a particular taxon in the plurality of taxa, and is not present among the group of sequences corresponding to that taxon, it may be added to the group of sequences corresponding to the taxon for use in later sequence comparisons. For example, if a bacterial genome is identified as belonging to a particular taxon, such as a genus or family, but the genome comprises a sequence that is not present in the sequences associated with that taxon, the bacterial genome may be added to the sequence database. Likewise, if the sample is derived from a particular source or condition, the sequencing read may be added to a reference database of sequences associated with that source or condition for use in identifying future samples that share the same source or condition. As a further example, a sequence that does not have a match at a lower level but does have a match at a higher level, as identified according to a method described herein, may be assigned to that higher level while also adding the sequencing read to the plurality of reference sequences that correspond to that taxonomic group. Reference databases so updated may be used in later sequence comparisons.

In determining the presence, absence or abundance of a taxon in a plurality of taxa (or polymorphism among a plurality of polymorphisms), two possible taxa may be tied for the assignment of a particular sequencing read. In such cases, the tie may be resolved. In one example, a tie is resolved by determining a sum of k-mer weights for the reference sequences along each branch of a phylogenetic tree connecting the taxa. The sequencing read may then be assigned to the node connected to the branch with the highest sum of k-mer weights.

A method may comprise determining the presence, absence, or abundance of a specific gene (e.g., antimicrobial resistant genes, antiviral resistant genes, antifungal resistant genes, antiprotozoal resistant genes, or antiparasitic resistant genes, etc.) or gene product (e.g., mRNA, protein product) within samples based on results of an earlier step. In this case, the plurality of reference polynucleotide sequences typically comprise groups of sequences corresponding to a gene in the plurality of genes. In some cases, at least 50, 100, 250, 500, 1000, 5000, 10000, 50000, 100000, 250000, 500000, or 1000000 different genes are identified as absent or present (and optionally abundance, which may be relative) based on sequences analyzed by a method described herein. In some cases, this analysis is performed in parallel. In some cases, the methods, compositions, and systems of the present disclosure enable parallel detection of the presence or absence of a gene in a community of genes, such as an environmental or clinical sample, when the gene identified comprises less than one per 10⁹, or one per 10⁶, or 0.05% of the total population of genes in the source sample. In some cases, detection is based on sequencing reads corresponding to a polynucleotide that is present at less than 0.01% of the total nucleic acid population. The particular polynucleotide may be at least 20%, 30%, 40%, 50%, 60%, 70%, 80%, 85%, 90%, 91%, 92%, 93%, 94%, 95%, 96% or 97% homologous to other nucleic acids in the population. In some cases, the particular polynucleotide is less than 75%, 50%, 40%, 30%, 20%, or 10% homologous to other nucleic acids in the population. Determining the presence, absence, or abundance of specific gene or gene product can comprise identifying an individual subject as the source of a sample. For example, a reference database may comprise a plurality of reference sequences, each of which corresponds to an individual organism (e.g. a human subject), with sequences from a plurality of different subjects represented among the reference sequences. Sequencing reads for an unknown sample may then be compared to sequences of the reference database, and based on identifying the sequencing reads in accordance with a described method, an individual represented in the reference database may be identified as the sample source of the sequencing reads. In such a case, the reference database may comprise sequences from at least 10², 10³, 10⁴, 10⁵, 10⁶, 10⁷, 10⁸, 10⁹, or more individuals.

In determining the presence, absence or abundance of a gene in a plurality of genes, two possible genes may be tied for the assignment of a particular sequencing read. In such cases, the tie may be resolved. In one example, a tie is resolved by determining a sum of k-mer weights for the reference sequences along each branch of a phylogenetic tree connecting the taxa pertaining to the associated gene. The sequencing read may then be assigned to the node connected to the branch with the highest sum of k-mer weights. In one example, a tie is resolved by determining.

In cases where a reference database consists of sequences associated with a condition, the method may comprise identifying the condition in the sample or the source from which the sample is derived. The condition may be identified based on the presence or change in 10%, 20%, 30%, 40%, 50%, 60%, 70%, 80%, 90%, or 100% of the components of a biosignature. Alternatively, a condition may be identified based on the presence or change in less than 20%, 10%, 1%, 0.1%, 0.01%, 0.001%, 0.0001%, or 0.00001% of the components of a biosignature. A sample may be identified as affected by the condition if at least, e.g., about 80% of the sequences and/or taxa associated with the condition are identified as present (or present at a level associated with the condition). A sample may be identified as affected by the condition if at least, e.g., about 80% of the sequences and/or genes associated with the condition are identified as present (or present at a level associated with the condition). The sample may be identified as affected by the condition if at least, e.g., at least about 90%, 95%, 99%, or more (e.g., all) sequences or taxa (or quantities of these) associated with the condition are present. A sample may be identified as affected by the condition if at least, e.g., about 90%, 95%, 99%, or more (e.g., all) sequences or genes (or quantities of these) associated with the condition are present. Where the condition is one of being from a particular individual, such as an individual subject (e.g. a human in a database of sequences from a plurality of different humans), identifying the sample as being affected by the condition comprises identifying the sample as being from the individual to whom the sequences in the database correspond. Identifying a subject as the source of the sample may be based on only a fraction of the subject's genomic sequence (e.g., less than about 50%, 25%, 10%, 5%, or less).

The presence, absence, or abundance of particular sequences, polymorphisms, genes (e.g., antimicrobial resistance, antiviral resistance, antivirulent resistance, antifungal resistance, antiparasitic resistance, antiprotozoal resistance, etc.), or gene products or taxa can be used for diagnostic purposes, such as inferring that a sample or subject has a particular condition (e.g. an illness), has had a particular condition, or is likely to develop a particular condition if sequence reads associated with the condition (e.g., from a particular disease-causing organism) are present at higher levels than a control (e.g., an uninfected individual). In another example, sequencing reads can originate from a host and indicate the presence of a disease-causing organism by measuring the presence, absence, or abundance of a host gene in a sample. In another example, the sequencing reads can originate from the host and indicate the presence of a disease-causing gene by measuring the presence, absence, or abundance of the gene in a sample. The presence, absence, or abundance can be used to determine the need for an intervention, such as a medical intervention and/or other treatment regimen, and details thereof. For example, the presence, absence, or abundance of a given microorganism or virus in a sample may inform a need for a medical intervention (e.g., medical treatment or care), inform the choice of a treatment regimen and the intensity and/or aggressiveness of the intervention, and provide insight into the effectiveness of a given treatment regimen and/or other intervention, where a decrease in the number of sequencing reads from a disease-causing agent during or after completion of a treatment regimen, or a change in the presence, absence, or abundance of specific host-response genes, indicates that a treatment regimen may be effective, whereas no change or insufficient change indicates that the treatment regimen may be ineffective. The sample may be assayed before or one or more times after treatment is begun. In some examples, the treatment of an infected subject may be altered based on the results of the monitoring. Identification of a pathogen or other element in a sample (e.g., in a subject from a sample) may also inform other interventions including practice interventions. Examples of such interventions may include how other people including visitors and medical personnel interact with a subject, including personal protective equipment (PPE) usage and potential quarantine recommendations; equipment and locations suitable for use in the care of a subject; and frequency and degree of cleaning of equipment and locations used in the care of a subject.

In some cases, one or more samples (e.g., blood, plasma, other body fluids, tissues, swab samples etc.) having a known condition may be used to establish a biosignature for that condition. The biosignature may be established by associating the record database with the condition. The biosignature may be established by associating the presence, absence, or abundance of the plurality of genes with the condition. The condition can be any condition described herein. For example, a plurality of samples from a particular environmental source may be used to identify sequences and/or taxa and/or genes associated with that environmental source, thereby establishing a biosignature consisting of those sequences and/or taxa so associated. In some cases, a plurality of samples from a particular environmental source may be used to identify sequences and/or genes associated with that environmental source, thereby establishing a biosignature consisting of those sequences and/or genes so associated. In general, the term “biosignature” is used to refer to an association of the presence, absence, or abundance of a plurality of sequences and/or taxa and/or genes with a particular condition, such as a classification, diagnosis, prognosis, and/or predicted outcome of a condition in a subject; a sample source; contamination by one or more contaminants; or other condition. A biosignature may be used as a reference database associated with a condition for the identification of that condition in another sample. Establishing the biosignature may comprise a determination of the presence, absence, and/or quantity of at least about 10, 50, 100, 1000, 10000, 100000, 1000000, or more sequences and/or taxa in a sample using a single assay. For example, establishing the biosignature may comprise a determination of the presence, absence, and/or quantity of at least 10, 50, 100, 1000, 10000, 100000, 1000000, or more sequences and/or genes in a sample using a single assay. Establishing a biosignature may comprise comparing sequencing reads for one or more samples representative of the condition with one or more samples not representative of the condition. For example, a biosignature can consist of gene expression involved in a host response (e.g., an immune response) among individuals infected by a virus, which sequences may be compared to sequences from subjects that are not infected or are infected by some other agent (e.g., bacteria). In such case, the presence, absence, or abundance of particular sequencing reads may be associated with a viral rather than a bacterial infection. In another example, the biosignature can consist of sequences of genes involved in a variety of antiviral responses, the presence, absence, or abundance of sequencing reads associated with which can be indicative of a specific class or type of viral infection. The biosignature associated with a reference database consists of the sequences (and optionally levels) of host transcripts and/or the sequences (and optionally levels) of transcripts or genomes of one or more infectious agents. In one particular example, the reference database could be common mutations or gene fusions found in cancerous cells, and the presence, absence, or abundance of sequencing reads associated with the biosignature can indicate that the patient has or does not have detectable cancer, what type of cancer a detectable cancer is, a preferred treatment method, whether existing treatment is effective, and/or prognosis.

Comparing sequences in accordance with a method provided herein can provide a variety of benefits. For example, computational resources used in the performance of a method may be substantially decreased relative to a reference method, such as a method based on traditional sequence alignment. For example, the speed with which a plurality of sequences in a sample are identified may be substantially increased. In some cases, identifying sequencing reads as corresponding to a particular reference sequence in a database of reference sequences may be completed for 10,000 or more sequence 20,000 or more sequences, 30,000 or more sequence, 40,000 or more sequence, 50,000 or more sequences, or 100,000 more sequence in less than 5 seconds, less than 4 seconds, less than 3 seconds, or less than 1 second of real time. In some cases, at least about 500000, 1000000, 2000000, 3000000, 4000000, 5000000, 10000000, or more sequences are identified per minute of real time. The set of sequences and processor used for benchmarking sequence identification processivity may be any that are described herein. In some cases, the sequencing reads used for benchmarking comprise sequences from two or more of bacteria, viruses, fungi, and humans. Performance of a method described herein may be defined relative to a reference tool, such as SURPI (see e.g. Naccache, S. N. et al. A cloud-compatible bioinformatics pipeline for ultrarapid pathogen identification from next-generation sequencing of clinical samples. Genome research 24, 1180-1192 (2014)) or Kraken (see e.g. Wood and Salzberg, “Kraken: ultrafast metagenomic sequence classification using exact alignments,” Genome biology 15, R46 (2014), which is hereby incorporated by reference). In some cases, a method of the disclosure is at least 5-fold, 10-fold, 50-fold, 100-fold, 250-fold or more rapid than SURPI in reaching results that are at least as accurate as SURPI using the same data set and computer hardware. In some cases, a method of the present disclosure provides improved accuracy relative to a reference analysis tool. For example, accuracy may be improved by at least 5%, 6%, 7%, 8%, 9%, 10%, 15%, 20%, 25%, or more, using the same data set and computer hardware. In some cases, sequences and/or taxa present in a known sample are identifies with an accuracy of at least about 80%, 85%, 90%, 95%, 96%, 97%, 98%, 99%, or higher. In some cases, the methods provided herein are operable to distinguish between two or more different polynucleotides based on only a few sequence differences. For example, methods provided herein may be utilized to distinguish between two or more strains of taxa (e.g. bacterial strains) based on a low degree of sequence variation between the compared taxa. In some cases, methods provided herein may be utilized to distinguish between two or more genes based on a low degree of sequence variation between the compared genes. In some cases, one or more taxa comprise a first bacterial strain identified as present and a second bacterial strain identified as absent based on one or more nucleotide differences in sequence (e.g. 1, 2, 3, 4, 5, 6, 7, 8, 9, 10, 25, 50, or more differences). In some cases, taxa are distinguished based on fewer than 25, 10, 5, 4, 3, 2, or fewer sequence differences. In some cases, the first bacterial strain is identified as present and the second bacterial strain is identified as absent based on a single nucleotide difference in sequence (e.g. a SNP). In some cases, one or more genes may comprise a first bacterial strain identified as present and a second bacterial strain identified as absent based on one or more nucleotide differences in sequence (e.g. 1, 2, 3, 4, 5, 6, 7, 8, 9, 10, 25, 50, or more differences). In some cases, genes may be distinguished based on fewer than 25, 10, 5, 4, 3, 2, or fewer sequence differences.

### Consensus Sequencing.

Consensus sequencing methods may be used to analyze sequences associated with a sample. A “consensus sequence,” as used herein, generally refers to a nucleotide sequence or amino acid sequence that is the calculated order of most frequent residues found at each position in a sequence alignment.

The sequence alignment may be as described elsewhere herein. In some cases, residues may be nucleotide(s) and/or amino acid(s). In some cases, the order of most frequent residues may be at least about 1 2, 3, 4, 5, 6, 7, 8, 9, 10, 50, 100, 1000, 10000 or more. In some cases, the order of most frequent residues may be at most about 10000, 1000, 100, 50, 10, 9, 8, 7, 6, 5, 4, 3, 2 or less. In some cases, the order of most frequent residues may be from about 1 to 10000, 1 to 1000, 1 to 100, 1 to 50, 1 to 10, 1 to 5 residues.

In some cases, a consensus sequence may be a sequence having similar structure in a different organism. In some cases, a consensus sequence may be a sequence of having similar function in different organisms. In some cases, a consensus sequence may be a sequence of having similar structure and function in different organisms. In some cases, the different organisms may be the same organism. In some cases, the different organism may be from different sample sources. In some cases, the different organism may be from the same sample source.

In some cases, a protein binding site may be represented by a consensus sequence. In some cases, a protein binding site consensus sequence may be a short sequence of nucleotides. In some cases, a protein binding site consensus sequence may be a short sequence of nucleotides which may be found several times in the genome.

In some cases, an average nucleotide identity may be a measure of nucleotide-level similarity. In some cases, an average nucleotide identity may be a measure of nucleotide-level similarity between regions of at least about 2, 3, 4, 5, 6, 7, 8, 9, 10, 50, 100, 1000 or more genomes. In some cases, an average nucleotide identity may be a measure of nucleotide-level similarity between regions of at most about 1000, 100, 50, 10, 9, 8, 7, 6, 5, 4, 3, 2 or less genomes. In some cases, an average nucleotide identity may be a measure of nucleotide-level similarity between regions from about 2 to 1000, 2 to 100, 2 to 50, 2 to 10, 2 to 5 genomes.

In some cases, an average nucleotide identity may be a measure of nucleotide-level similarity between sample sources. In some cases, an average nucleotide identity may be a measure of nucleotide-level similarity between of at least about 2, 3, 4, 5, 6, 7, 8, 9, 10, 50, 100, 1000 sample sources. In some cases, an average nucleotide identity may be a measure of nucleotide-level similarity between at most about 1000, 100, 50, 10, 9, 8, 7, 6, 5, 4, 3, 2 or less sample sources. In some cases, an average nucleotide identity may be a measure of nucleotide-level similarity between about 2 to 1000, 2 to 100, 2 to 50, 2 to 10, 2 to 5 sample sources

In some cases, an average nucleotide identity may be a measure of nucleotide-level similarity between a sample source and a reference sequence. In some cases, an average nucleotide identity may be a measure of nucleotide-level similarity between at least about 1, 2, 3, 4, 5, 6, 7, 8, 9, 10, 50, 100, 1000 sample sources and at least about 1, 2, 3, 4, 5, 6, 7, 8, 9, 10, 50, 100, 1000 reference sequences.

In some cases, an average nucleotide identity may be a measure of nucleotide-level similarity between at most about 1000, 100, 50, 10, 9, 8, 7, 6, 5, 4, 3, 2 sample sources and at most about 1000, 100, 50, 10, 9, 8, 7, 6, 5, 4, 3, 2 reference sequences.

In some cases, a sequence alignment may be a way of arranging sequences to identify a consensus sequence. In some cases, the sequence alignment may be a way of arranging sequences to identify regions of similarity that may be a consequence of a relationship between the sequences. In some cases, the sequences may be from, for example, DNA, RNA, or protein, etc. In some cases, the regions of similarity may be a consequence of functional, structural, and/or evolutional relationships between sequences. In some cases, the consensus sequence may represent the results of multiple sequence alignments.

In some cases, aligned sequences of nucleotide and/or amino acid residues may be represented as rows within a matrix. In some cases, gaps may be inserted between the residues. In some cases, gaps may be inserted between the residues so that identical and/or similar characters may be aligned in successive columns.

In some cases, if two sequences in an alignment share a common ancestor, mismatches may be interpreted as point mutations. In some cases, if two sequences in an alignment share a common ancestor, mismatches may be interpreted as point mutations introduced in one or both lineages in the time since they diverged from one another.

In some cases, if two sequences in an alignment share a common ancestor, gaps may be interpreted as indels (e.g., insertion and/or deletion mutations). In some cases, if two sequences in an alignment share a common ancestor, gaps may be interpreted as indels (e.g. insertion and/or deletion mutations) introduced in one or both lineages in the time since they diverged from one another.

In some cases, the sequence alignments may be of proteins. In some cases, the degree of similarity between amino acids of proteins occupying a particular position in the sequence may be interpreted as a measure of how conserved a particular region or sequence motif is among lineages. In some cases, the absence of substitutions between two sequence alignments in a particular region of the sequence may suggest that this region has structural and/or functional importance. In some cases, the presence of only very conservative substitutions (that is, the substitution of amino acids whose side chains have similar biochemical properties) in a particular region of the sequence may suggest that this region has structural and/or functional importance. In some cases, the conservation of base pairs (e.g. base pairs of DNA nucleotide bases, base pairs of RNA nucleotide bases) may indicate a similar functional and/or structural role.

In some cases, the method may perform overlap detection of sequences. In some cases, the method may use an algorithm. The algorithm may be, for example, a greedy algorithm on a suffix tree. The use of a greedy algorithm on a suffix tree may allow a wide-range of specific matches and errors. The use of a greedy algorithm on a suffix tree may provide flexibility and/or sensitivity in overlapping reads of widely disparate lengths and/or error patterns (e.g. hybrid assembly of long reads from one sequencing platform with short reads from a different platform).

In some cases, the method may facilitate identification of overlap regions in sequence data having high insertion and/or deletion rates relative to substitution rates, e.g., using modified k-mer error models and/or modified suffix tree query algorithms.

In some cases, the method may use a parallelized version of the AMOS layout algorithm Tigger. In some cases, the method may use a parallelized version of the AMOS layout algorithm Tigger and a consensus algorithm. In some cases, the consensus algorithm may employ a probabilistic graphical model to represent the error characteristics of long reads.

In some cases, the method may further refine a sequence alignment construct. In some cases, simulated annealing and/or nontraditional objective functions may be used for alignment refinement. In some cases, alignment refinement may comprise the use of global chaining in combination with sparse dynamic programming.

In some cases, the method may be a computer-implemented method. The computer-implemented method may identify regions of sequence overlap between a plurality of sequencing reads. In some cases, the method may comprise providing the plurality of sequencing reads within a data structure. In some cases, the method may generate a set of k-mers having deletions and/or insertions. In some cases, the method may search the data structure for regions of the sequencing reads that match a first k-mer of the set of k-mers. In some cases, the regions may be identified as regions of sequence overlap between the sequencing reads. In some cases, the method may search the data structure with further k-mers in the set of k-mers to identify further regions of sequence overlap between the sequencing reads. In some cases, the set of k-mers may include both deletion-comprising k-mers and/or insertion-comprising k-mers, k-mers having multiple deletions, k-mers having multiple insertions, k-mers having substitutions, or combinations thereof.

In some cases, the set of k-mers may have a combined insertion-deletion rate of about 1% to about 40%. In some cases, the set of k-mers may have a combined insertion-deletion rate of about 1% to about 5%, about 1% to about 10%, about 1% to about 15%, about 1% to about 20%, about 1% to about 25%, about 1% to about 30%, about 1% to about 35%, about 1% to about 40%, about 5% to about 10%, about 5% to about 15%, about 5% to about 20%, about 5% to about 25%, about 5% to about 30%, about 5% to about 35%, about 5% to about 40%, about 10% to about 15%, about 10% to about 20%, about 10% to about 25%, about 10% to about 30%, about 10% to about 35%, about 10% to about 40%, about 15% to about 20%, about 15% to about 25%, about 15% to about 30%, about 15% to about 35%, about 15% to about 40%, about 20% to about 25%, about 20% to about 30%, about 20% to about 35%, about 20% to about 40%, about 25% to about 30%, about 25% to about 35%, about 25% to about 40%, about 30% to about 35%, about 30% to about 40%, or about 35% to about 40%. In some cases, the set of k-mers may have a combined insertion-deletion rate of about 1%, about 5%, about 10%, about 15%, about 20%, about 25%, about 30%, about 35%, or about 40%. In some cases, the set of k-mers may have a combined insertion-deletion rate of at least about 1%, about 5%, about 10%, about 15%, about 20%, about 25%, about 30%, or about 35%. In some cases, the set of k-mers may have a combined insertion-deletion rate of at most about 5%, about 10%, about 15%, about 20%, about 25%, about 30%, about 35%, or about 40%.

In some cases, the set of k-mers may be stored and/or searched for in a data structure, e.g., a hash table, a suffix tree, a suffix array, or a sorted list. In some cases, the data structure may be searched using a greedy algorithm. In some cases, the data structure may be searched using a greedy algorithm modified to allow for k-mers having mutations, such as insertions, deletions, and substitutions. In some cases, the data structure may be searched using an O(N) algorithm. In some cases, the data structure may be searched using an O(N) algorithm comprising Bloom filters. In some cases, the Bloom filters may optionally store the set of k-mers.

In some cases, providing the sequencing reads may comprise performing at least about one sequencing-by-incorporation assay. In some cases, providing the sequencing reads may comprise performing about 1 to about 1000 sequencing-by-incorporation assays. In some cases, providing the sequencing reads may comprise performing about 1 to about 5, about 1 to about 10, about 1 to about 25, about 1 to about 50, about 1 to about 100, about 1 to about 1000, about 5 to about 10, about 5 to about 25, about 5 to about 50, about 5 to about 100, about 5 to about 1000, about 10 to about 25, about 10 to about 50, about 10 to about 100, about 10 to about 1000, about 25 to about 50, about 25 to about 100, about 25 to about 1000, about 50 to about 100, about 50 to about 1000, or about 100 to about 1000 sequencing-by-incorporation assays. In some cases, providing the sequencing reads may comprise performing about 1, about 5, about 10, about 25, about 50, about 100, or about 1000 sequencing-by-incorporation assay. In some cases, providing the sequencing reads may comprise performing at least about 1, about 5, about 10, about 25, about 50, or about 100 sequencing-by-incorporation assays. In some cases, providing the sequencing reads may comprise performing at most about 5, about 10, about 25, about 50, about 100, or about 1,000 sequencing-by-incorporation assays.

In some cases, the sequencing-by-incorporation assay may be performed in a confined reaction volume. In some cases, the confined reaction volume may be a zero-mode waveguide.

In some cases, redundant sequencing methods may include resequencing and/or sequencing multiple copies of a template molecule. In some cases, redundant sequencing methods may be used to generate the sequencing reads. In some cases, the sequencing reads may be filtered, e.g., before being included in the data structure, and such filtering can be performed on the basis of various criteria including, but not limited to, read quality and/or call quality. In some cases, one or more of the plurality of sequencing reads, the data structure, the set of k-mers, the regions of sequence overlap, and/or the further regions of sequence overlap may be stored on a computer-readable medium and/or displayed on a screen as described elsewhere herein.

In some cases, the method may identify regions of sequence overlap between sequencing contigs. In some cases, the method may derive a plurality of first sequencing contigs from a first plurality of sequencing reads. In some cases, the method may derive a plurality of first sequencing contigs from a first plurality of sequencing reads from a first sequencing method.

In some cases, the method may derive a second plurality of second sequencing contigs from a second plurality of sequencing reads. In some cases, the method may derive a second plurality of second sequencing contigs from a second plurality of sequencing reads from a second sequencing method. In some cases, the first and second sequencing methods may be different from one another. In some cases, the first and second sequencing methods may be the same. In some cases, the method may incorporate the first sequencing contigs and/or the second sequencing contigs into a data structure.

In some cases, the method may generate a set of k-mers. In some cases, the method may search the data structure for regions of the sequencing contigs that match a first k-mers of the set of k-mers. In some cases, the regions may be identified as regions of sequence overlap between the first sequencing contigs and the second sequencing contigs. In some cases, the method may repeat the searching with further k-mers in the set of k-mers. In some cases, the method may repeat the searching with further k-mers in the set of k-mers to identify further regions of sequence overlap between the first sequencing contigs and the second sequencing contigs. In some cases, the set of k-mers may be optionally stored and/or searched for in a data structure, e.g., a hash table, a suffix tree, a suffix array, or a sorted list. The data structure may be searched using various algorithms, e.g., a greedy algorithm and/or an O(N) algorithm. The various algorithms may comprise Bloom filters. In some cases, the Bloom filters may optionally store the set of k-mers. In some cases, at least one of the first or second sequencing method may be a sequencing-by-incorporation method. In some cases, at least one of the sequencing contigs, the data structure, the set of k-mers, the regions of sequence overlap, and the further regions of sequence overlap may be stored on a computer-readable medium and/or is displayed on a screen as described elsewhere herein.

In some cases, the first plurality of sequencing reads may be long. In some cases, the first plurality of sequencing reads may be contiguous. In some cases, the sequencing reads and the second plurality of sequencing reads may be short and/or paired-end sequencing reads.

In some cases, the method may identify regions of sequence overlap between sequencing contigs. In some cases, the method may further comprise deriving a plurality of third sequencing contigs from a third plurality of sequencing reads from a third sequencing method. In some cases, third sequencing method may be different from the first and second sequencing methods. In some cases, the method may incorporate the third sequencing contigs into the data structure. In some cases, the regions identified during the searching may be regions of sequence overlap between the first sequencing contigs, the second sequencing contigs, and the third sequencing contigs. In some cases, the first and second sequencing methods may be selected from pyrosequencing, tSMS sequencing, Sanger sequencing, Solexa sequencing, SMRT sequencing, SOLID sequencing, Maxam and Gilbert sequencing, nanopore sequencing, and semiconductor sequencing.

In some cases, the method may align a sequence read to a reference sequence. In some cases, the method may comprise mapping short subsequences of the sequence read to the reference sequence. In some cases, the method may comprise mapping short subsequences of the sequence read to the reference sequence using, for example, a suffix array, a global chaining, identifying regions within the reference sequence to which a plurality of the subsequences of the sequence read map, scoring and remapping the regions using sparse dynamic programming, and/or aligning matches, e.g., using basecall quality values and at least one of a banded affine or pair-HMM, alignment. In some cases, the scoring and mapping may be performed iteratively.

In some cases, a sequence read may be provided. A sequence read may be provided by performing a sequencing reaction on a target nucleic acid. A reference sequence for the target nucleic acid may be provided and a set of subsequences in the sequence read may identified. In some cases, a set of subsequences in the sequence read may identified if each of the subsequences match a portion of the reference sequence. The set of subsequences may be refined, optionally iteratively, by scoring and realigning the subsequences to the reference sequence. The set of subsequences may be refined, optionally iteratively, by scoring and realigning the subsequences to the reference sequence using sparse dynamic programming. A banded dynamic programming alignment, e.g., affine or Pair-HMM, may be used to score and realign the final set of subsequences to provide the final alignment of the sequence read to the reference sequence.

In some cases, the identification of the matching subsequences may comprise finding all exact matches from the sequence read that may be longer than a minimum match length, k, and that match the reference sequence. In some cases, the identification of the subsequences in the sequence read that match portions of the reference sequence may be performed using a suffix array and/or a BWT-FM index. In some cases, the identification of the subsequences in the sequence read that match portions of the reference sequence may comprise clustering exact matches using global chaining. The clustering may comprise sorting the exact matches by position within the reference sequence and within the sequence read. The clustering may comprise sorting the exact matches by position within the reference sequence and within the sequence read and finding a first subset of non-overlapping exact matches that may be larger than any other subset of non-overlapping exact matches. In some cases, the first subset may be identified as a cluster and the cluster may one of the set of subsequences. In some cases, the set of subsequences may be scored and ranked prior to the refining steps. In some cases, following the scoring and/or realigning, each iteration of the refining redetermines subsets of non-overlapping exact matches. In some cases, the method may further identify the largest of these subsets. In some cases, the banded alignment may comprise aligning all bases in the sequence read to the reference sequence using alignments from the sparse dynamic programming as a guide. In some cases, a mapping quality value may be preferably calculated. In some cases, various steps of the method may be implemented on a computer, e.g., using computer-readable code, and various results or outputs from the steps can be stored on computer-readable media and/or displayed on a computer monitor as described elsewhere herein.

In some cases, a system may be configured to generate a consensus sequence. In some cases, the system may comprise computer memory. In some cases, the computer memory may comprise a sequence read for a target nucleic acid. In some cases, the computer memory may comprise a reference sequence for the target nucleic acid. In some cases, the computer memory may comprise a computer-readable code for finding a set of subsequences in the sequence read that match portions of the reference sequence. In some cases, the computer memory may comprise computer-readable code for refining the set of subsequences. In some cases, refining comprises scoring and/or realigning the subsequences may use sparse dynamic programming. In some cases, the computer memory may comprise computer-readable code for scoring and realigning a final set of subsequences using a banded alignment. In some cases, the banded alignment may align the sequence read to the reference sequence. In some cases, computer memory may be configured to store the output of at least one of the steps of the method. In some cases, the system may comprise a monitor for displaying at least one of the sequence read, the reference sequence, and/or the output of at least one of the steps of the method as described elsewhere herein.

In some cases, a system may be configured to generate a consensus sequence. In some cases, the system may comprise computer memory. The computer system may comprise a sequence read for a target nucleic acid. In some cases, the computer memory may comprising a reference sequence for the target nucleic acid. In some cases, the system may comprise computer-readable code for finding a set of subsequences in the sequence read that match portions of the reference sequence. In some cases, the computer-readable code may refine the set of subsequences. In some cases, refining comprises scoring and realigning the subsequences using sparse dynamic programming. In some cases, the computer-readable code for scoring and realigning a final set of subsequences may use a banded alignment. In some cases, the banded alignment may align the sequence read to the reference sequence. In some cases, the computer memory may be configured to store the output of at least one of the steps of the method. In some cases, the system may comprise a monitor for displaying at least one of the sequence read, the reference sequence, and the output of at least one of the steps of the method as described elsewhere herein.

In some cases, a system may be configured to generate a consensus sequence. In some cases, the system comprises computer memory. In some cases, the computer memory may contain a set of sequence reads; computer-readable code for applying an overlap detection algorithm to the set of sequence reads and generating a set of detected overlaps between pairs of the sequence reads; computer-readable code for assembling the set of sequence reads into an ordered layout based upon the set of detected overlaps; and memory for storing the ordered layout.

In some cases, the method may identify periodicity for a repetitive sequence read. The method may comprise calculating a self-alignment scoring matrix. In some cases, the method may comprise calculating a self-alignment scoring matrix with a special boundary condition for the repetitive sequence read. In some cases, the method may sum over the scoring matrix to generate a plot. In some cases, the plot may provide accumulated matching scores over a range of base pair offsets. In some cases, the method may identify a set of peaks in the plot having highest accumulated matching scores. In some cases, the method may determine a first base pair offset for a first peak in the set. In some cases, the first peak may have a lower base pair offset than any of the other peaks. In some cases, the method may identify the periodicity for the repetitive sequence read as an amount of the first base pair offset. In some cases, the method may determine at least a second base pair offset for a second peak in the set. In some cases, the second peak may have a lower base pair offset than any of the other peaks except the first peak. In some cases, the method may use the second base pair offset to validate the first base pair offset. In some cases, the periodicity for the repetitive sequence read determined by the methods herein may be used during overlap detection within the repetitive sequence read.

In some cases, the method may analyze sequence information. In some cases, the method may analyze the assembly of overlapping sequence data into a contig. In some cases, the method may determine a consensus sequence. In some cases, the methods may analyze sequences of biomolecular sequences, such as nucleic acids, amino acids, polypeptides, or proteins, etc.

In some cases, the method may provide de novo assembly and consensus sequence determination through analysis of biomolecular (e.g. nucleic acid, polypeptide, amino acids, etc.) sequence data.

In some cases, the method may comprise a first step for sequence analysis. The first step may comprise determining one or more sequence reads, or contiguous orders of the molecular units, or monomers in the sequence. For example, a nucleic acid sequencing read may comprise an order of nucleotides or bases in a polynucleotide, e.g., a template molecule and/or a polynucleotide strand complementary thereto. In some cases, the determination of sequence reads that can be analyzed by the methods provided herein include, e.g., Sanger sequencing, shotgun sequencing, pyrosequencing (454/Roche), SOLID sequencing (Life Technologies), ISMS sequencing (Helicos), Illumina R sequencing, and in certain preferred cases, single-molecule real-time (SMRT™) sequencing (Pacific Biosciences of California).

In some cases, for each type of sequencing technology, experimental data collected during one or more sequencing reactions may be analyzed to determine one or more sequence reads for a given template nucleic acid subjected to the sequencing reaction(s). For example, pyrosequencing may rely on production of light by an enzymatic reaction following an incorporation of a nucleotide into a nascent strand that may be complementary to a template nucleic acid. In some cases, fluorescently-labeled oligonucleotides may be detected during SOLID sequencing. In some cases, fluorescently-labeled nucleotides may be used in tSMS, Illumina R, and SMRT sequencing reactions. In some cases, in SMRT sequencing, a set of differentially labeled nucleotides, template nucleic acid, and a polymerase may be present in a reaction mixture. As the polymerase processes the template nucleic acid a nascent strand may be synthesized that may be complementary to the template nucleic acid. The label on each nucleotide may be linked to a portion of the nucleotide that may not be incorporated into the nascent strand. The labeled nucleotides in the reaction mixture may bind to the active site of the polymerase enzyme. In some cases, during the binding and subsequent incorporation of the constituent nucleoside monophosphate, the label may be removed and may diffuse away from the complex. In some cases, the label may be linked to the terminal phosphate group of the nucleotide. In some cases, the label may be cleaved from the nucleotide by the enzymatic activity of the polymerase which cleaves the polyphosphate chain between the alpha and beta phosphates. In some cases, since detection of fluorescent signal may be restricted to a small portion of the reaction mixture that includes the polymerase, e.g., within a zero-mode waveguide (ZMW), a series of fluorescence pulses may be detectable and may be attributed to incorporation of nucleotides into the nascent strand with the particular emission detected being indicative of a specific type of nucleotide (e.g., A, G, T, or C). In some cases, by analyzing various characteristics of the pulse trace, which may comprise a series of detected fluorescence pulses, the sequence of nucleotides incorporated can be determined and, by complementarity, the sequence of at least a portion of the template nucleic acid may be derived therefrom. The identification of the type and order of nucleotides incorporated may be performed using computer-implemented methods.

In some cases, different sequencing technologies may have different inherent error profiles in the sequence reads they produce. In some cases, redundancy in the sequence data may be used to identify and/or correct errors in individual sequence reads. Various methods may be used to produce sequence data having such redundancy. For example, the reactions can be repeated, e.g., by iteratively sequencing the same template, or by separately sequencing multiple copies of a given template. In doing so, multiple reads may be generated for one or more regions of the template nucleic acid. In some cases, each read overlaps completely or partially with at least one other read in the data set produced by the redundant sequencing. In some cases, different regions of a template can be sequenced by using different primers to initiate sequencing in different regions of the template. In some cases, the resulting sequence reads may overlap to allow construction of a consensus sequence representative of the true sequence of the different regions of the template nucleic acid based upon sequence similarity between portions of different reads that overlap within those regions

In some cases, the sequence reads for a given template sequence may be assembled as described elsewhere herein. In some cases, the sequence reads for a given template sequence may be assembled like a puzzle based upon sequence overlap between the reads, e.g., to form a contig. In some cases, the alignment of the reads relative to one another may provide the position of each read relative to the other reads. In some cases, the alignment of the reads relative to one another may provide the position of each read relative to the template nucleic acid. In some cases, longer and/or more accurate reads facilitate contig assembly. In some cases, a known reference sequence (e.g., from a public database or repository, or as described elsewhere herein) can also be used during construction of the contig. In some cases, a region that may be covered by two or more individual sequence reads having overlapping segments corresponding at least to the region may be subjected to a more accurate sequence determination. In some cases, the overlapping portions of the sequence reads that correspond to the region may be compared or otherwise analyzed with respect to one another. In some cases, erroneously called bases may be identified and, optionally corrected, in individual reads during the assembly process. In some cases, this information may be used to determine a more accurate consensus sequence for the region. In some cases, once the alignment between separate reads is determined, a best or most likely call can be determined for each position in the overlapping portions, assigned to that position in a consensus sequence, and used to determine the most likely call for that position in the original template molecule.

In some cases, a consensus sequence determination for a template molecule may be facilitated by accurate alignments of the overlapping sequencing reads. In some cases, accurate alignments of the overlapping sequencing reads may allow determination of which positions within individual reads correspond to a single position in the template sequence. In some cases, certain sequence read characteristics may complicate alignment. For example, some sequencing technologies may produce very short sequence reads, which require a very high fold-coverage to ensure the template sequence is adequately covered. In some cases, even at high fold-coverage these reads may not allow resolution of highly repetitive regions, e.g., that are longer than the typical length of the reads. In some cases, other sequencing technologies may produce long sequencing reads that allow better resolution of repeat regions and facilitate assembly, but may do so at the expense of accuracy. In some cases, the types of errors that characterize sequence reads may be substitutions (e.g., misincorporation or miscalled bases) versus insertions and deletions (e.g., multiply-counted or missed bases).

In some cases, the method provides alignment of individual sequence reads with one another, e.g., for the purposes of identifying regions of overlap between the sequence reads. In some cases, identifying regions of overlap between the sequence reads may be useful in determining an accurate sequence of a template molecule. In some cases, identifying regions of overlap between the sequence reads may be useful in determining an accurate sequence of a template molecule that was subjected to the sequencing reaction. In some cases, different types of sequence reads can be combined into a single contig, or into a scaffold. In some cases, different types of sequence reads can be combined into a single contig, or into a scaffold, which may include positions for which a base call has not been determined (e.g., that correspond to gaps in the raw sequence reads), which can be designated by “N” in the scaffold. For example, less accurate long sequence reads may be combined with short but more accurate sequence reads using the hybrid assembly method, as further described elsewhere herein. The long reads may facilitate placement of the small reads into a contig or scaffold, and the basecalls in the short reads may be given more weight in the final consensus sequence determination due to their higher inherent accuracy. In some cases, the advantages inherent to each type of sequence read can be used to maximize the accuracy of the resulting assembly.

In some cases, the methods may use BLASR (Basic Local Alignment with Successive Refinement). In some cases, the method may use BLASR that may use a combination of data structures in short read mapping with sparse dynamic programming alignment methods. In some cases, A BWT-FM index or suffix array of a genome may be queried to generate short exact matches that may be clustered. In some cases, the method may give approximate starting and ending coordinates in the genome for where a read should align. In some cases, a more detailed alignment may be generated by using sparse dynamic programming between a set of short exact matches in the read to the region it maps to. In some cases, a final detailed alignment may be generated using dynamic programming within an area guided by the sparse dynamic programming alignment.

In some cases, a method may align and assemble nucleic acid sequencing reads.

In some cases, the nucleic acid sequencing reads may comprise overlapping or redundant sequence information. In some cases, the method may be used in combination with other alignment and assembly methods as described elsewhere herein. For example, the overlap detection may comprise one or more alignment algorithms that align each read using a reference sequence. In some cases, a reference sequence may be known for a region containing the target sequence, the reference sequence may be used to produce an alignment using a variant of the center-star algorithm. In some cases, the sequence alignment may comprise one or more alignment algorithms that may align each read relative to every other read without using a reference sequence (e.g. de novo assembly routines), e.g., PHRAP, CAP, ClustalW, T-Coffee, AMOS make-consensus, or other dynamic programming MSAs.

In some cases, a method may align and assemble sequence reads based at least in part on a known reference sequence. In some cases, aligning and assembling sequence reads may be based at least in part on a known reference sequence. In some cases, aligning and assembling sequence reads based at least in part on a known reference sequence may be resequencing or mapping as described elsewhere herein. In some cases, the sequence reads may be mapped to the reference sequence. In some cases the sequence reads may be mapped to the reference sequence, and loci that may have base calls that differ from the reference sequence may be further analyzed to determine if a given locus was erroneously called in the sequence read, and/or if it may represent a true variation (e.g., a mutation, SNP variant, etc.). In some cases, the variation may distinguish the nucleotide sequence of the reference sequence from that of the template nucleic acids that were sequenced to generate the sequence reads. In some cases, variations may encompass multiple adjacent positions in the reference and/or the sequencing reads, e.g., as in the case of insertions, deletions, inversions, or translocations. In some cases, a sequence may be assembled based upon the alignment of the reference sequence and the sequence reads that are similar but not necessarily identical to at least a portion of the reference sequence.

In some cases, a method may align and assemble sequence reads that do not use a known reference sequence. In some cases, aligning and assembling sequence reads may be termed used in de novo sequencing. In some cases, the sequence reads may be analyzed to identify overlap regions. In some cases, the sequence reads may be aligned to each other to generate a contig. In some cases, the contig may be subjected to consensus sequence determination, e.g., to form a new, previously unknown sequence, such as when an organism's genome may be sequenced for the first time. In some cases, de novo assemblies may be orders of magnitude slower. In some cases, de novo assemblies may have more memory intensive than resequencing assemblies. In some cases, de novo assemblies may need to analyze or compare every read with every other read, e.g., in a pair-wise fashion. In some cases, the sequence reads themselves may be used as reference in the alignment algorithms.

In some cases, a method may perform a hybrid assembly of nucleic acid sequencing reads. In some cases, the method may assemble long (e.g., those generated by Pacific Biosciences™ SMRT™ sequencing (“PacBio reads”)) and short (e.g., those generated by Illumina R) nucleic acid sequencing reads. In some cases, a method for hybrid assembly may take reads from different sequencing methodologies and align them with each other. In some cases, more and longer sequence reads may facilitate identification of sequence overlaps. In some cases, more and longer sequence reads may have higher error rates than reads from short-read technologies. In some cases, short sequence reads may be faster to align. In some cases, short sequence reads may be more difficult to align when the template from which they were generated comprises repeats (identical or near-identical) or large rearrangements, such as inversions or translocations, that are longer than the length of the short reads. In some cases, longer reads from a first platform may be used to form a baseline to which other types of reads, e.g., from short-read platforms, may be added. In some cases, the method may allow sequencing data from the different platforms to be combined to provide overall higher quality data, e.g. due to higher redundancy or compensation of one or more weaknesses of one with the strengths of the other. In some cases, a hybrid assembly can be used to select regions of high quality reads from one platform based on the higher quality sequence generated by another other platform.

In some cases, a method may use a hybrid assembly for de novo assembly. In some cases, overlaps in hybrid assemblies may be augmented or filtered in various ways. For example, candidate overlap regions observed in the long reads may be corroborated with regions in the short reads that overlap the candidate overlap regions in the long reads. In some cases, candidate overlap regions between long reads or long and short reads may be corroborated if they are flanked or spanned by a mate pair or strobe reads. In some cases, corroboration of a candidate overlap may be accomplished by comparison to a reference sequence. In some cases, regions that do not align to a reference sequence may be targeted for more aggressive mis-assembly detection. In some cases, analysis of experimental sequence read data may override the reference sequence (which may contain sequence data that does not correspond to the template sequence, e.g., due to genetic variability, errors in reference sequence determination, etc.).

In some cases, the method may comprise de novo assembly. In some cases, the de novo assembly may comprise a first step. In some cases, the first step may be overlap detection. In some cases, overlap detection may be performed in a pairwise fashion. In some cases, two sequence reads may be compared and/or analyzed with respect to one another at a time. In some cases, the process may continue until all sequence reads have been compared to all other sequence reads. In some cases, de novo assembly may comprise a second step. In some cases, the second stage may be layout, in which the overlaps detected in the first stage may be used to order all the sequence reads having such overlaps with respect to one another. In some cases, de novo assembly may comprise a third step. In some case, the third step may be consensus sequence determination, in which positions within the overlapping regions that may be different within different reads may be further analyzed to determine a best call for the position, e.g., based upon quality scores for individual basecalls and the frequency of each type of basecall within the set of sequence reads that include that position. In some cases, de novo assembly may produce assembled reads, or contigs. In some cases, de novo assembly may provide the best sequence for the template nucleic acid from which the sequence reads were derived.

In some cases, a method for hybrid assembly may comprise an overlap determination step. In some cases, a method for hybrid assembly may comprise a layout step. In some cases, a method for hybrid assembly may comprise consensus sequence determination step. In some cases, the input sequences may be have high confidence reads or contigs from multiple different sequencing technologies, e.g., short-read and long-read technologies. In some cases, the different sequencing technologies used in hybrid assembly may produce sequence reads and/or contigs having different error profiles, e.g., that may be characterized by different types and/or frequencies of sequencing and/or assembly errors. In some cases, the process may assemble the contigs (e.g., FASTA-formatted) from the different technologies to produce hybrid contigs or scaffolds, which may be presented as oriented contigs in a linear graph (for example, in FASTA or graphml format). In some cases, depending on the types of reads used in the hybrid assembly process, the resulting linear graphs may contain ambiguous regions or gaps, e.g., where one or more positions are not covered by the assembled contigs. For example, in some cases the original sequence reads may not include the positions within the gap, and in other cases the quality of calls within the gap region may be determined to be too low to include these calls in the hybrid assembly process.

In some cases, a method for hybrid assembly may be used for error correction within reads of one sequencing technology using the reads from a second sequencing technology. For example, errors within reads from an error-prone, long-read sequencing technology may be corrected using reads from a low-error, short-read sequencing technology. In some cases, such an error correction assembly method may carried out as follows: for an N number of iterations, an alignment may be performed using a sequence read from the sequencing technology having a lower raw accuracy and a set of sequence reads from the sequencing technology having a higher raw accuracy. In some cases, the sequence read may have a longer read length. In some cases, BLASR, may be used as an alignment method. In some cases, the alignment output may be converted to a SAM file format and SAMTOOLS may be used to generate a pileup formatted version of the MSA. In some cases, the pileup file may be used for error correction. In some cases, the pileup file may include, for example, the position at which a correction is being made, the number of reads from the more accurate sequencing technology that covered that position, the base that was previously present at that position, the type of error correction event (e.g., deletion, insertion, substitution), the corrected base, the consensus base, and the PHRED score of the corrected base. In some cases, for each read base position recorded in the pileup, the consensus call generated may be accepted or rejected according to (a) the number of more accurate reads used in determining the consensus call, (b) the percentage of consensus agreement amongst the more accurate reads, and (c) the PHRED value of the majority-called base. In some cases, a summary of the accepted consensus calls may be generated. In some cases, a summary of the accepted consensus calls may be used to create an updated sequence read for the less accurate sequencing technology. In some cases, the updated sequence read may be stored and, optionally, subjected to a further iteration of the alignment and error correction method (“correction iteration”) to generate a further updated sequence. In some cases, once all iterations are complete, an overall summary of all error corrections incorporated into the sequence read from the less accurate sequencing technology may be generated. In some cases, the pileup step may be optimized by selecting areas within the read to correct rather than correcting the entire read. In some cases, selection of such areas may be guided by the results of former correction iterations.

In some cases, a method for de novo assembly may comprise a number of steps.

In some cases, the first step may be determining overlap between reads. In some cases, the second step may be laying out overlapping reads in a linear order by aligning the overlap regions with one another for the set of reads that may overlap with at least one other read. In some cases, the third step may be construction of a final consensus from the oriented read.

In some cases, the overlap component, regions of sequence similarity between sequence reads may be identified. The assembly process may assume that such regions of overlap originate from the same place within the template nucleic acid. In some cases, once the overlap regions have been identified, the sequence reads may be laid out such that the overlap regions are aligned with one another. In some cases, most or all of the template nucleic acid may be represented in the set of sequence reads so aligned. In some cases, in the consensus step, a consensus basecall may be determined for each position in the template nucleic acid based upon the set of sequence reads that comprise each position. For example, where all basecalls are identical over the set of sequence reads, the basecall may be become the consensus basecall. In some cases, where there are different basecalls in different sequence reads, a best basecall may be determined based on various criteria, including but not limited to the quality of that basecall in each individual sequence read the frequency of each type of basecall over the set of sequence reads. In some cases, the process can be iterative, e.g., to further refine the consensus sequence. In some cases, the method for de novo assembly of sequence reads may have a high insertion-deletion rate, e.g., over a 5%, or a 10%, or a 15%, or in some cases up to a 20% error rate. In some cases, a greedy suffix tree may detect overlaps using sequence reads having accuracies of about 80%. In some cases, algorithms using Bloom filters may detect overlaps using sequence reads having accuracies of only about 85%.

In some cases, the input to assembly construction may be a set of sequence reads generated from a single template nucleic acid sequence (e.g., via redundant sequencing of one or more template molecules and/or sequencing of identical template molecules). In some cases, the outputs may include a set of pair-wise overlaps, a layout or contig comprising the sequence reads comprising regions represented in the pair-wise overlaps, and/or a single consensus sequence that best represents the nucleotide sequence present in the original template nucleic acid sequence or the complement thereof, etc. In some cases, the assembly process may generate a set of overlaps. In some cases, the set of overlaps may be used to align a set of sequence reads to form a contig. In some cases, the set of overlaps may be analyzed to determine a single consensus sequence. In some cases, the production of a consensus sequence may be important for a wide variety of further analyses of the sequence determined for the template, e.g., in identifying sequence variants, performing a functional analysis based upon homology to known genes or regulatory sequences, or comparing it to other sequences to determine evolutionary relationships between different species, subspecies, or strains, etc.

In some cases, a method for de novo assembly may be derived from the AMOS assembler, which is an open-source, whole-genome assembler available from the AMOS consortium. In some cases, method may use a mixture of python and C/C++, as well as SWIG bindings to AMOS libraries. In some cases, SWIG may a tool that simplifies the integration of C/C++ with common scripting languages. In certain cases, a filtering step may be included between the consensus step and the terminate assembly decision. In some cases, the Amos CTG may feed into this filtering step. In some cases, in the filtering step, contigs with low coverage or a small number of reads may be filtered out. In some cases, the contigs may be filtered out because these contigs may be due to low-frequency error sequences, such as chimeras. In some cases, the final scaffolding step may not performed. In some cases, the final scaffolding step may be replaced instead with the hybrid assembly methods described herein.

In some cases, a method for de novo overlap detection may comprise a pairwise analysis of the sequence reads in the original data set to determine regions of overlap between pairs of individual reads. In some cases, this step may be computationally expensive. In some cases, for large genomes may involve the comparison of millions of individual reads (for potentially trillions of pair-wise comparisons). In some cases, sequence assembly algorithms may apply rapid filters to determine read pairs that are likely to overlap. For example, various methods of filtering and trimming the data may be used, for example, vector trimming, quality filtering, length filtering, no call read filtering, low complexity filtering, shadow read filtering, read trimming, or end trimming, etc.

In some cases, the determination of sequence assembly may also involve analysis of read quality (e.g., using TraceTuner™, Phred, etc.), signal intensity, peak data (e.g., height, width, shape, proximity to neighboring peak(s), etc.), information indicative of the orientation of the read (e.g., 5′→3″ designations), clear range identifiers indicative of the usable range of calls in the sequence, and the like. In some cases, such read quality may be used to exclude certain low quality reads from the alignment process. In some cases, not every call in each read is used in the overlap detection process. In some cases, high raw error rates may indicate a benefit to selecting only reads with a high quality (e.g., high certainty). For example, the quality of the calls in each read may be measured and only those identified as high quality may be used in the alignment process. In some cases, a position may not be included in the overlap detection operation if at least a portion of the calls for that position in replicate sequences are below a quality criteria. In some cases, the quality of a given call may be dependent on many factors. In some cases, the quality of a given call may be related to the sequencing technology being used. For example, factors that may be considered in determining the quality of a call include signal-to-noise ratios, power-to-noise ratio, signal strength, trace characteristics, flanking sequence (“sequence context”), and known performance parameters of the sequencing technology, such as conformance variation based on read length. In some cases, the quality measure for the observed call may be based, at least in part, on comparisons of metrics for such additional factors to metrics observed during sequencing of known sequences. Methods and software for generating sequence calls and the associated quality information is widely available. For example, PHRED is one example of a base-calling program that may output a quality score for each call. After the set of pairwise overlaps has been generated, the calls of lower quality may be added back to the alignment, or, optionally may be kept out of the assembly process altogether, or may be added back at a later stage.

In some cases, after a set of pair-wise overlaps has been identified by an overlap-detection method, each overlap may be assigned a score. In some cases, scores allow discrimination between correct and incorrect overlaps. In some cases, a score threshold may set such that a very small number of overlaps that exceed this threshold may be incorrect. In some cases, a score threshold may set such that a very small number of overlaps that exceed this threshold may be incorrect and all overlaps below this threshold are ignored. In some cases, a score may be the results of Smith-Waterman alignment of the two sequences. In some cases, additional methods of overlap scoring methods may be used as described elsewhere herein.

In some cases, detecting overlaps may be to search for regions of exact match between the sequence reads, e.g., subsequent to the filtering described elsewhere herein. In some cases, exact matches may be detected using simple lookup tables, hashing functions, or more complicated structures such as overlapping algorithms such as the suffix tree. In some cases, suffix trees may have the advantage of rapid creation and query lookup time, (O(n) and O(l), respectively, where n is the size of the database). In some cases, the method may modify the suffix tree query algorithms to create a greedy suffix tree overlap algorithm that may allow for insertions and deletions. In some cases, the greedy suffix may maintain the suffix tree's desirable creation and query time.

In some cases, the input to a method may comprise two sets of FASTA-formatted sequences, a query and a target. In some cases, FASTA format is a widely used text-based format for representing either nucleotide or peptide sequences using single-letter codes to represent nucleotides or amino acids. In some cases, a compressed suffix tree may be created from the target sequences. In some cases, each query sequence may be subsequently compared with the suffix tree using a greedy algorithm. In some cases, a greedy algorithm may attempt to find the shortest common supersequence given a set of sequence reads by calculating pairwise alignments of all sequence reads; choosing two reads with the largest overlap; merging the two chosen reads; and repeating the steps until only one merged read remains. In some cases, the method may return matches that obey two user-specified parameters, m the minimum number of matched nucleotides, and e the maximum number of errors. In some cases, an error is an insertion or deletion between the query and target sequence. In some cases, for high error rate data, e can be quite large relative to m (e.g., e=35, m=80).

In some cases, the greedy algorithm may alternate between two modes. In some cases, in the first mode it may attempt to exactly match as much of the query sequence as possible against the target suffix tree. In some cases, after further exact matches are impossible, the greedy algorithm may enters a second mode. In some cases, the second mode may introduce errors in the query sequence (e.g., substitutions, insertions, or deletions). In some cases, after each introduced error, the greedy algorithm may return to the first mode, greedily attempting to exactly match as much of the (now modified) query sequence as possible. In some cases, the greedy algorithm may continue to alternate between the two modes until it terminates. In some cases, the greedy algorithm may terminate when it has matched a certain threshold or more characters from the query, or it has been forced to introduce at least a certain number of errors.

In some cases, the greedy algorithm may not an exhaustive overlap detection algorithm. In some cases, the greedy algorithm may not find all matches that satisfy the constraints m and e. In some cases, the number of matches returned for a particular query sequence can be increased by starting the greedy algorithm at different positions along the query, for example, every 10 bases. In some cases, the algorithm may be used within the context of an iterative assembly, in which overlaps may be detected at multiple stages, allowing algorithm to catch overlaps it missed in previous iterations and to avoid generating overly fragmented assemblies.

In some cases, the greedy algorithm may be used with data structures other than the suffix tree. In some cases, other data structures such as a hash or lookup tables could be used. In some cases, as compared to the suffix tree, the suffix array consume less memory, but may have a longer query time. In some cases, the hash and lookup table-based methods may suffer from reduced spatial locality of reference when introducing errors in the sequence. In some cases, the suffix array may provide better locality of reference properties than the suffix tree, with proper caching schemes.

In some cases, the greedy suffix tree overlap algorithm may be used during de novo assembly. In some cases, the greedy suffix overlap algorithm may be used to map an observed sequence read to a known or candidate target sequence (e.g., generated based upon the sequence reads themselves). In some cases, a suffix tree may be constructed from a target database (e.g., FASTA or pls.h5). In some cases, a query database (database containing the sequence read data) may be aligned to this tree using a greedy suffix tree algorithm. In some cases, the tree alternates between two modes: 1) exact match of the query to the tree; and 2) mutation of query. In some cases, the algorithm greedily accepts the longest match, which can include up to a specified number of errors. In some cases, the results may be checked with banded Smith-Waterman algorithm. In some cases, the results may be outputted in AMOS OVL messages.

In some cases, sequence alignment may be performed using an approach of successive refinement to map single molecule sequencing reads. In some cases, the algorithm that may be used to carry out this successive alignment process is termed a Basic Local Alignment via Successive Refinement (BLASR) algorithm. In some cases, this algorithm may be understood as having two basic steps: 1) find high-scoring matches of a read in the reference sequence (which may be derived from the sequence reads in de nova assembly) genome, and 2) refine matches until the homologous sequence to the read is found in the reference sequence. In some cases, the first step may involve matching short subsequences or suffices of an observed sequence read to a reference sequence using a suffix array (based on short read mapping methods).

In some cases, short-read aligners may use Burrows-Wheeler Transform (BWT) String for searching.

In some cases, the second step of BLASR may use global chaining to find high-scoring sets of anchors. In some cases, the resulting putative matches may be scored using Sparse Dynamic Programming. In some cases, the matches may be aligned using a Pair-Hidden Markov Model with quality values in called bases.

In some cases, the BLASR method may have any number of steps. In some cases, the BLASR algorithm may detect candidate intervals by clustering short exact matches. In some cases, the BLASR algorithm may approximate alignment of reads to candidate intervals using sparse dynamic programming. In some cases, the BLASR algorithm may detail banded alignment using the sparse dynamic programming alignment as a guide. In some cases, read base positions may be assigned to reference positions during the detail banded alignment.

In some cases, the method for determining overlaps between sequence data may involve identification of small regions of exact matches using k-mers between reads. In some cases, sequences that share a large number of k-mers may come from the same region of the sequence to be identified, e.g., a genomic sequence. In some cases, the value of k may be the length of the matched region. In some cases, the value of k may be the length of the matched region and may be on the order of 20-30 base pairs. In some cases, these regions can be found rapidly using data structures such as suffix trees or hash tables. In some cases, for two overlapping reads to share an exactly k-mer, the two reads may either have low error rates and/or be sufficiently long to compensate for the high chance of errors. In some cases, for sequencing reads having relatively frequent errors, the method may be modified to allow errors in the k-mers.

In some cases, a gapped k-mer method may provide an insertion-deletion tolerance of detecting potential overlap between reads. For example, when searching for matches to k-mer in a particular read, the algorithm enumerates all k-d-mers that can be created from that k-mer by introducing d deletions. In some cases, for example, if the original k-mer is ATGC (k=4) and the desired number of deletions is 1 (d=1), the method may produce four 3-mers, each with a missing base or gap at one of the four positions in the original 4-mer: TGC, AGC, ATC, and ATG. In some cases, the method may allow for insertions or substitution.

In some cases, the method may have several parameters that may be varied or altered. In some cases, for example, the length of the k-mer; the number of insertions, deletions, or substitutions, if any; the data structure in which the k-mers are found (hash tables, suffix tree, suffix array, or sorted list); and whether gapped k-mers are stored explicitly or merely searched for implicitly in these data structures can be changed or adjusted. In some cases, the optimal value of each of these parameters may be dependent on the characteristics of the genome being sequenced and computational resources available for assembly.

In some cases, Bloom filters may be used in an O(N) algorithm to determine pairs of sequences with matching overlaps in order to decrease the run time and accelerate the analysis. In some cases, the algorithm may provide greater than 100-fold increases in analysis speed without any significant loss in sensitivity. In some cases, the Bloom filter may be used to store the set of all sequence read identifiers from a given analysis for sequences that contain a particular feature. In some cases, an identifier Bloom filter may be constructed for every potential feature, and may be used to determine candidate read pairs that share a large number of features. In some cases, the features may be the presence or absence of a particular k-mer (gapped or ungapped) in the sequence.

In some cases, the method inputs may be two files of sequence reads, a query and a target, which can be the same file or two or more different files. In some cases, a Bloom filter may be created for each possible k-mer. In some cases, each Bloom filter may contain m bits, where m may be on the order of two to ten times the number of sequences expected to possess each feature. In some cases, the target sequence database may be scanned in linear time, processing target sequences in turn. In some cases, each sequence identifier may be encoded by h hash functions (e.g., h=2), and converted into a value between 0 and m. In some cases, for each k-mer in the sequence, the h bits corresponding to the hashed values of the sequence identifier may be set in that k-mer's Bloom filter. In some cases, a compact representation of the presence of absence of each k-mer in every read in the target database may be constructed.

In some cases, the Bloom filters may be interrogated using each query sequence, again in linear time. In some cases, each query sequence may be converted into a set of k-mers, and the Bloom filters for each of these k-mers may be subsequently summed. In some cases, the bits that are set a large number of times in this Bloom filter sum may correspond to hashed values for sequence identifiers that share a large number of k-mers with the query sequence. In some cases, an inverse hash that maps the h hashed values of each sequence identifier may be used to retrieve the target identifiers for this particular query.

In some cases, the method comprising Bloom filters may have a running time of O(N). In some cases, some of the fundamental operations, such as constructing the Bloom filters, querying them, and summing the resulting Bloom filters, may be readily parallelized. In some cases, depending on the size of the sequence to be aligned, the identifier Bloom filters may require large amounts of memory during the analysis. In some cases, an alignment may be subsequently checked using a Smith-Waterman alignment algorithm. In some cases, larger assemblies (such as the human genome) may require more memory. In some cases, a target database of size G may use a Bloom filter representation of 2G to 10G. In some cases, chunking may be used to facilitate the analysis of larger assemblies, e.g., if distributed across multiple nodes.

In some cases, the method may contain at least two free parameters that may be modified while preserving the objective of determining overlap regions between sequence reads. In some cases, the first may be the number of bits stored in each Bloom filter (in). In some cases, increasing this value may increase the sensitivity of the algorithm. In some cases, this may increase the memory consumption. In some cases, the second parameter may be the number of hash functions used to encode sequence read identifications (h). This value may be as low as 1 or as high as m−1. Increasing h can either increase or decrease sensitivity, depending on the value of m and the average number of bits set in a particular Bloom filter. In some cases, there may be a much wider family of algorithms that involve using features other than k-mer presence or absence to construct the identifier Bloom filters. In some cases, some may be closely related to the k-mer concept, but may be deconstructed after the sequence has been transformed in some wa. For example, one transformation may be to collapse all homopolymers before k-mer identification. In some cases, may convert all GCs into ones and all ATs into zeroes. In some cases, a class of features completely unrelated to k-mer presence may summarize the entire sequence in some way, such as using the presence or absence of high GC content.

In some cases, steps may be taken to maximize efficiency during the overlap detection operation, e.g., to reduce the occurrence of both duplicate comparisons and missed comparisons.

In some cases, some sequence reads may comprise redundant sequence information. For example, a nucleic acid molecule can be repeatedly sequenced in a single sequencing reaction to generate multiple sequence reads for the same template molecule, e.g., by a rolling-circle replication-based method. In some cases, a concatemeric molecule comprising multiple copies of a template sequence can be subjected to sequencing-by-synthesis to generate a long sequence read comprising multiple complements to the copies. In some cases, when a circular or concatemeric molecule is used as a template for iterative or redundant sequencing, the final sequence read should have a periodic structure. For example, when a circular template is repeatedly processed by a polymerase enzyme, such as in a rolling-circle replication, a long sequencing read may be generated that comprises multiple complements of the template, which can be referred to as sibling reads. In some cases, the periodic pattern can be difficult to identify in certain circumstances, e.g., when using a template of unknown sequence (e.g., size and/or nucleotide composition) and/or when the resulting sequence data contains miscalls or other types of errors (e.g., insertions or deletions).

In some cases, the template may comprise a known sequence that can be used to align the multiple sibling reads within the overall redundant sequencing read with one another and/or with a known reference sequence. In some cases, the known sequence may be an adaptor that may be linked to the template prior to sequencing, or may be a partial sequence of the template, e.g., where the partial sequence was used to pull down a particular region of a genome from a complex genomic sample. In some cases, by identifying the locations of the alignments between multiple occurrences of the known sequence within the sequencing read, one may infer the periodicity of the read.

In some cases, the template does not comprise a known sequence that can be reliably aligned to deduce the periodicity. In some cases, this can be accomplished by aligning the sequencing read to itself and finding self-similar patterns using standard alignment algorithms,

In some cases, a whole self-alignment score matrix may be used to calculate a quantity that is analogous to the autocorrelation for continuous signal. This autocorrelation function may be used to infer periodicity for discrete sequences with high insertion and/or deletion error rates. In some cases, the information of the whole self-alignment score matrix may be used to estimate the periodicity of the sequence. In some cases, the self-alignment scoring matrix may be calculated using a special boundary condition, which can be adjusted depending on the known characteristics of the sequencing data and/or the template from which it was generated. In some cases, the self-alignment score matrix may comprise summing over the scoring matrix for all different lags. In some cases, the self-alignment score matrix may comprise identifying the peaks and their periodicity used to infer the periodicity of the sequence data. In some cases, the self-alignment score matrix may comprise using the periodicity of the sequence data to guide self-alignment of the sibling reads within the sequence data.

In some cases, in order to reveal non-zero offset self-alignment, a special boundary condition may be imposed that forces all of the diagonal elements of the scoring matrix to be zero. In some cases, this may prevent the zero-offset self-alignment from contributing to the scoring matrix. In some cases, without this boundary condition, the contribution of the zero-offset self-alignment may occlude or mask out the non-zero-offset self-alignment.

In some cases, a spatial genome assembler may be provided. In some cases, sequences may be treated as character strings and string-matching techniques may be used to identify overlap between reads to combine short reads into longer ones. In some cases, the method may map DNA reads into an N-space coordinate system such that any given length of DNA becomes an N-dimensional thread through space.

In some cases, the method may use associations between sibling reads generated from the same template molecule to improve overlap detection for de novo assembly. In some cases, assembly methods may combine sibling reads into a single consensus read using a consensus sequence discovery process. In some cases, the sibling reads may be analyzed without consensus sequence determination, but while still taking into account their relationship as multiple reads of the same template sequence. In some cases, the method can be extended to mapping of reads to a reference sequence or any method that assigns information to a particular sibling read that can be usefully shared among its siblings.

In some cases, summation may be used to share overlap score information among sibling reads. In some cases, overlaps may be initially called or identified between reads using an alignment algorithm, such as one of those described elsewhere herein. In some cases, scores for pairs of reads that belong to the same group of siblings (e.g., were generated from the same template molecule) may be combined by summing the scores. In some cases, combining overlap scores across sibling reads may provide dramatic improvements in the true positive rate, demonstrating that more overlaps are correctly detected, even in the presence of varying error rates and false positive rates. In other cases, other methods of combining scores may be used, e.g., max, min, product.

In some cases, the method may use multiple sequence alignment (MSA) to establish homology relationships between a set of three or more sequences, e.g., nucleotide or amino acid sequences. In some cases, multiple sequence alignments may be used to construct phylogenetic trees, understand structure-sequence relationships, highlight conserved sequence motifs, and of particular relevance to the sequencing methods provided herein, provide a basis for consensus sequence determination given a set of sequencing reads from the same template.

In some cases, the method provides an MSA refinement procedure using Simulated Annealing and a different objective function. In some cases, a simulated annealing framework may be used to search and evaluate the solution space.

In some cases, the initial alignment may be a close approximation of the optimal solution. In some cases, each new candidate alignment may be generated by making a local perturbation of the current alignment. In some cases, the alignment may disrupt by randomly selecting a column in the MSA and performing a gap shifting operation with some probability for each sequence having a gap in that column. In some cases, gap shifts may occur to the right or to the left of the current column. In some cases, each new candidate may be evaluated using the GeoRatio objective function (a geometric ratio objective function), which scores an alignment block.

In some cases, the scoring mechanism may compute the geometric mean of the signal-to-noise ratio within a column, where a column is a set of calls for a given position in the assembled reads. For example, in nucleotide sequence data, a column can be the set of basecalls for a nucleotide position overlapped by a plurality of assembled sequencing reads, where each read provides one of the basecalls.

In some cases, the new candidate alignment may be accepted if its score is better than the current solution and accepted with some probability if the score is worse. In some cases, bad trades may occasionally be made in order to prevent the algorithm from sinking into a local optimum. In some cases, the temperature used at each iteration of the process can be set using an exponential decay function, and the chance with which you may accept a bad solution decreases as the temperature cools. In some cases, after making the decision to accept or reject the candidate, the process either stops (if termination criteria are met) or proceeds to the next iteration. In some cases, termination criteria are met when n iterations have passed without improvement or after exceeding a predefined number of iterations.

In some cases, to assess the result of MSA refinement, consensus calling accuracy at low coverage (2-6×) may be compared. In some cases, the alignment problem may be made more difficult and realistic by mutating the reference at every 500th position to a random yet different base. In some cases, the mutated reference (represents the re-sequencing reference) may be used for read alignment and initial MSA construction. In some cases, the original reference (represents the sample) may be used for consensus sequence comparison. In some cases, this MSA refinement improves low coverage consensus calling.

### Identification of Anti-Microbial Resistance Genes.

The present disclosure provides systems and methods for determining the presence, absence, or abundance of specific genes within samples (e.g., based on results of an earlier step, as described herein). In this case, the plurality of reference polynucleotide sequences typically comprise groups of sequences corresponding to individual genes in the plurality of genes. In some cases, at least 50, 100, 250, 500, 1000, 5000, 10000, 50000, 100000, 250000, 500000, or 1000000 different genes are identified as absent or present (and optionally abundance, which may be relative) based on sequences analyzed by a method described herein. In some cases, this analysis is performed in parallel. In some cases, the methods, compositions, and systems of the present disclosure may enable parallel detection of the presence or absence of a gene in a community of genes, such as an environmental or clinical sample, when the gene is identified comprises less than 0.05% of the total population of genes in the source sample. In some cases, detection is based on sequencing reads corresponding to a polynucleotide that is present at less than 0.01% of the total nucleic acid population. The particular polynucleotide may be at least 20%, 30%, 40%, 50%, 60%, 70%, 80%, 85%, 90%, 91%, 92%, 93%, 94%, 95%, 96% or 97% homologous to other nucleic acids in the population. In some cases, the particular polynucleotide is less than 75%, 50%, 40%, 30%, 20%, or 10% homologous to other nucleic acids in the population. Determining the presence, absence, or abundance of specific taxa can comprise identifying an individual subject as the source of a sample. For example, a reference database may comprise a plurality of reference sequences, each of which corresponds to an individual organism (e.g. a human subject), with sequences from a plurality of different subject represented among the reference sequences. Sequencing reads for an unknown sample may then be compared to sequences of the reference database, and based on identifying the sequencing reads in accordance with a described method, an individual represented in the reference database may be identified as the sample source of the sequencing reads. In such a case, the reference database may comprise sequences from at least 10², 10³, 10⁴, 10⁵, 10⁶, 10⁷, 10⁸, 10⁹, or more individuals.

In some cases, identifying the presence, absence, or abundance of a gene or plurality of genes may be used to diagnose a condition based on a degree of similarity between the gene or plurality of genes detected in the sample and a biological signature for the condition.

The presence, absence, or abundance of genes can be used for diagnostic purposes, such as inferring that a sample or subject has a particular condition (e.g. an illness) if sequence reads from a particular disease-causing gene are present at higher levels than a control (e.g. an uninfected individual). In an example, the sequencing reads can originate from the host and indicate the presence of a disease-causing gene by measuring the presence, absence, or abundance of a host gene in a sample. The presence, absence, or abundance can be used to infer effectiveness of a treatment, where a decrease in the number of sequencing reads from a disease-causing agent after treatment, or a change in the presence, absence, or abundance of specific host-response genes, indicates that a treatment is effective, whereas no change or insufficient change indicates that the treatment is ineffective. The sample can be assayed before or one or more times after treatment is begun. In some examples, the treatment of the infected subject is altered based on the results of the monitoring.

The present disclosure provides methods for identifying one or more pertaining anti-microbial resistance genes pertaining to a sample source. The sample source may be as described elsewhere herein. In some cases, the method may compare sequencing reads for a plurality of protein amino acid sequences to a database of reference protein amino acid sequences.

The matching of empirical sequencing data to the references for the AMR gene may be at the level of protein amino acids. In some cases, the matching of empirical sequencing data to the references for the AMR gene may be at the level of nucleotide sequences.

The method may produce a bit score result. In some cases, the bit score result may be the weighting of the matching output between the plurality of protein amino acid sequences and the reference protein amino acid sequences.

The antimicrobial resistant genes may be associated with a bacterial pathogen as described elsewhere herein. An anti-microbial resistance gene may be a gene that may allow an organism to resist the mechanism with certain antibiotics. In some cases, an anti-microbial resistance gene may be a gene of an organism that may resist the effects of medication. In some cases, the anti-microbial resistance gene may be a gene of an organism that may resist the effects of medication that once successfully treated the organism. In some cases, the antimicrobial resistant genes may be unique for a particular bacterial strain, or shared by several bacterial strains. Examples of antimicrobial resistance genes include, but are not limited to, penicillin-resistance genes, tetracycline-resistance genes, streptomycin-resistance genes, methicillin-resistance genes, and glycopeptide drug-resistance genes. In some cases, the genes which confer resistance to antibiotics may be present on plasmids in a cell. In some cases, in order for an organism to produce the factor which confers resistance, the gene for the factor and the mRNA for the factor must be present in the cell. In some cases, a probe specific for the factor mRNA can be used to detect, identify, and quantitate the organisms from the sample source which are producing the factor.

### Read Alignments.

Upon identification of k-mers and, in some cases, other sequences or components thereof within a given set of sequencing reads, k-mers and sequencing reads may be aligned to identify species or other entities with which they may be associated. Read alignment may comprise alignment of reads, including reads that have been identified as being components of a same sequence, against one or more reference sequences, including one or more reference sequences from a reference database (e.g., as described herein).

Read alignments may be performed with high accuracy and precision. In some cases, read alignment accuracy may exceed 60%, such as at least 65%, 70%, 75%, 80%, 85%, 90%, 95%, 99%, or higher. Read alignment may comprise quantitative assessment of sequences, and therefore associated entities, within a given sample. In some cases, quantitative analysis of entities within a sample may have accuracy of at least 60%, such as at least 65%, 70%, 75%, 80%, 85%, 90%, 95%, 99%, or higher. As described herein, controls may be used to facilitate read alignment and quantitative analysis.

Read alignments may be analyzed to provide metrics regarding coverage and identity of species. For example, read alignments may be used to identify species within a given sample. Sequence coverage for given species may also be analyzed. Such information may be fed back into the classification module to facilitate future process and/or analysis improvements, including improved curation of reference database and/or sample preparation.

### Detection.

A detection module, which may be operatively coupled to a classification module, may be used to identify entities (e.g., species) within a given sample. In silico validation via a classification algorithm (e.g., as described herein) may optionally be coupled with variable stringency to apply cutoffs or machine learning approach to identified reads or contigs toward identifying entities (e.g., species) within a given sample. Where specific markers are of interest, logic may be applied to facilitate the markers' identification. Putative organism and, where of interest, marker identification may then be performed.

Diagnostic calls (positive or negative) for each organism may be made by programmatically applying validated cutoffs or machine learning approaches to metrics generated by a classification algorithm. These cutoffs may be specific to each organism and be on a gradient in which one set of cutoffs is highly stringent for specificity while another set allows for specificity. Such cutoffs may be validated by exhaustively running simulations and/or controls through a classification algorithm. The presence or absence of specific markers, including anti-microbial resistance (AMR) markers, may be determined using essentially the same algorithm as for organism detection, plus logic that may limit the occurrence of certain markers (e.g., AMR markers) to defined subsets of organisms known to harbor these specific markers.

A detection module may be a component of a classification module. Like a classification module, a detection module may include a display and/or interface with which a user may interact. For example, a user may apply and/or alter cutoffs for read analysis, select specific markers of interest, etc.

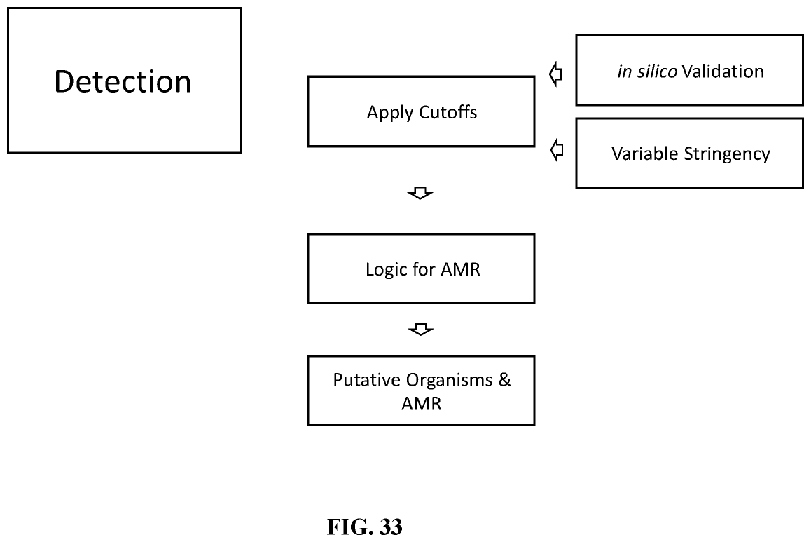

An example detection module is schematically illustrated in FIG. 33.

As further illustrated in FIG. 33, the list of identified identities from in silico validation can be pruned so that only those entities that have specific markers (e.g., AMR markers) are selected. Further still, as illustrated in FIG. 33, the list of identified entities can be further pruned against one or more selected diagnostic test profile(s) so only those entities that also match criteria of particular selected diagnostic test profile(s) are retained. As an example, consider the case where the selected diagnostic test profile is limited to human disease. In this instance, only those entities (species) that are associated with human disease are retained. As an example, consider the case where the selected diagnostic test profile is limited to chicken pox. In this instance, only those entities (species) that are associated with chicken pox are retained.

As such, FIG. 33 illustrates a computer system, methods, and computer readable memory that obtain, in electronic form, a set of sample sequence reads for a plurality of polynucleotides from the first sample, wherein the set of sequence reads comprises at least 50,000 sequence reads. For each respective sequence read in the set of sample sequence reads or a respective sample contig derived from a respective subset of the set of sample sequence reads, there is performed a corresponding sequence comparison between at least a portion of the respective sample sequence read or respective sample contig and each reference sequence in a first set of reference sequences thereby performing a first plurality of sequence comparisons.

Optionally comparison are performed against any number of additional sets of reference sequences. For instance optionally performing, for each respective sequence read in the set of sample sequence reads or a respective sample contig derived from a respective subset of the set of sample sequence reads, a corresponding sequence comparison between at least a portion of the respective sample sequence read or respective sample contig and each reference sequence in a second set of reference sequences thereby performing a second plurality of sequence comparisons.

There is calculated from the first plurality of sequence comparisons, and any additional comparisons performed, a respective probability that the respective sample sequence read or the respective sample contig corresponds to a particular reference sequence in the first, or optionally additional, set(s) of reference sequences thereby computing a first plurality of probabilities.

A plurality of candidate species based at least in part on the first plurality of probabilities is found. Examples of how this is done are disclosed herein and also in U.S. patent application Ser. No. 15/724,476, entitled “Methods and Systems and Multiple Taxonomic Classification,” filed Oct. 4, 2017, which is hereby incorporated by reference.

As illustrated in FIG. 33, there is removed from the plurality of candidate species those candidate species that fail to include specific marker (e.g., an anti-microbial resistance marker) thereby forming a set of one or more species and identifying a presence or an absence of one or more species in the first sample as the set of one or more species.

As further illustrated in FIG. 33, in some embodiments, the set of one or more species is filtered against one or more of the diagnostic test profiles disclosed herein (e.g., that have been selected by a user) such that those species in the set of one or more species that fail to be associated with one or more diseases specified by the one or more diagnostic test profiles are removed from the set of one or more species.

In some embodiments, only a single diagnostic test profile is selected. In such embodiments the set of one or more species is filtered against a single diagnostic test profile such that those species in the set of one or more species that fail to be associated with a disease specified by the single diagnostic test profiles are removed from the set of one or more species.

### Systems for Sequence Identification.

The present disclosure provides systems for performing any of the methods described herein. A system may be configured for identifying a plurality of polynucleotides in a sample from a sample source based on sequencing reads for the plurality of polynucleotides. For example, the system may comprise a computer processor programmed to, for each sequencing read: (a) perform a sequence comparison between the sequencing read and a plurality of reference polynucleotide sequences, where the comparison comprises calculating k-mer weights as measures of how likely it is that k-mers within the sequencing read are derived from a reference sequence within the plurality of reference polynucleotide sequences; (b) identify the sequencing read as corresponding to a particular reference sequence in a database of reference sequences if the sum of k-mer weights for the reference sequence is above a threshold level; and (c) assemble a record database comprising reference sequences identified in step (b), where the record database excludes reference sequences to which no sequencing read corresponds. As another example, the system may comprise one or more computer processors programmed to: (a) for each sequencing read, perform a sequence comparison between the sequencing read and a plurality of reference polynucleotide sequences, where the comparison comprises calculating k-mer weights as measures of how likely it is that k-mers within the sequencing read are derived from a reference sequence within the plurality of reference polynucleotide sequences; (b) for each sequencing read, calculate a probability that the sequencing read corresponds to a particular reference sequence in a database of reference sequences based on the k-mer weights, thereby generating a sequence probability; (c) calculate a score for the presence or absence of one or more taxa based on the sequence probabilities corresponding to sequences representative of said one or more taxa; and (d) identify the one or more taxa as present or absent in the sample based on the corresponding scores.

The system may further comprise a reaction module in communication with the computer processor, where the reaction module performs polynucleotide sequencing reactions to produce the sequencing reads. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules or techniques which, in turn, may be implemented in hardware, firmware, software, or any combination thereof. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc. In some cases, the computer is configured to receive a customer request to perform a detection reaction on a sample. The computer may receive the customer request directly (e.g. by way of an input device such as a keyboard, mouse, or touch screen operated by the customer or a user entering a customer request) or indirectly (e.g. through a wired or wireless connection, including over the internet). Non-limiting examples of customers include the subject providing the sample, medical personnel, clinicians, laboratory personnel, insurance company personnel, or others in the health care industry.

The present disclosure also provides a computer-readable medium comprising codes that, upon execution by one or more processors, may implement a method according to any of the methods disclosed herein. Execution of the computer readable medium may implement a method of identifying a plurality of polynucleotides in a sample from a sample source based on sequencing reads for the plurality of polynucleotides. The execution of the computer readable medium may implement a method comprising: (a) for each of the sequencing reads, performing a sequence comparison between the sequencing read and a plurality of reference polynucleotide sequences, where the comparison comprises calculating k-mer weights as measures of how likely it is that k-mers within the sequencing read are derived from a reference sequence within the plurality of reference polynucleotide sequences; (b) for each of the sequencing reads, identifying the sequencing read as corresponding to a particular reference sequence in a database of reference sequences if the sum of k-mer weights for the reference sequence is above a threshold level; and (c) assembling a record database comprising reference sequences identified in step (b), where the record database excludes reference sequences to which no sequencing read corresponds.

The execution of the computer readable medium may implement a method of identifying one or more taxa in a sample from a sample source based on sequencing reads for a plurality of polynucleotides, the method comprising: (a) for each of the sequencing reads, performing a sequence comparison between the sequencing read and a plurality of reference polynucleotide sequences, where the comparison comprises calculating k-mer weights as a measure of how likely it is that k-mers within the sequencing read are derived from a reference sequence within the plurality of reference polynucleotide sequences; (b) for each of the sequencing reads, calculating a probability that the sequencing read corresponds to a particular reference sequence in a database of reference sequences based on the k-mer weights, thereby generating a sequence probability; (c) calculating a score for the presence or absence of one or more taxa based on the sequence probabilities corresponding to sequences representative of said one or more taxa; and (d) identifying the one or more taxa as present or absent in the sample based on the corresponding scores.

The execution of the computer readable medium may implement a method of identifying one or more genes in a sample from a sample source based on sequencing reads for a plurality of polynucleotides. The method may comprise: (a) for each of the sequencing reads, performing a sequence comparison between the sequencing read and a plurality of reference polynucleotide sequences, where the comparison comprises calculating k-mer weights as a measure of how likely it is that k-mers within the sequencing read are derived from a reference sequence within the plurality of reference polynucleotide sequences; (b) for each of the sequencing reads, calculating a probability that the sequencing read corresponds to a particular reference sequence in a database of reference sequences based on the k-mer weights, thereby generating a sequence probability; (c) calculating a score for the presence or absence of one or more genes based on the sequence probabilities corresponding to sequences representative of said one or more genes; and (d) identifying the one or more genes as present or absent in the sample based on the corresponding scores.

Computer readable medium may take many forms, including but not limited to, a tangible storage medium, a carrier wave medium, or physical transmission medium. Non-volatile storage media include, for example, optical or magnetic disks, such as any of the storage devices in any computer(s) or the like, such as may be used to implement the calculation steps, processing steps, etc. Volatile storage media include dynamic memory, such as main memory of a computer. Tangible transmission media include coaxial cables; copper wire and fiber optics, including the wires that comprise a bus within a computer system. Carrier-wave transmission media can take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media may be involved in carrying one or more sequences of one or more instructions to a processor for execution.

### Interpretation

A method or system of the present disclosure may comprise an interpretation module. An interpretation module may enable user interaction with sequencing data and classification information. An interpretation module may comprise software and a user interface that presents sequencing data and/or classification information with textual and/or visual indicators including reports that may be viewed, accessed, downloaded, uploaded, or otherwise interacted with. Interpretation software may generate one or more reports that may be outputtable for interpretation by a user, such as a medical professional, laboratory technician, research scientist, or other user. A report may be formatted as, e.g., a portable document format (PDF) file and/or JavaScript object notation (JSON) format. Depending on the classification and detection processes employed, which may relate to particular classes of infections and entity targets (e.g., respiratory tract infections, urinary tract infections, ascites/abdominal infections, blood infections, central nervous system infections, joint infections, and sexually transmitted infections), detected entities (e.g., organisms) may be categorized as pathogens or as part of normal flora based on, e.g., published studies and/or reference databases. For each broad category of entities, such as bacteria, fungi, parasite, and virus, a report may provide an estimate of the proportion of each detected pathogen relative to all detected entities of that category. A report may also indicate the analytical sensitivity of the results based on analysis of control organisms used in the laboratory process.

An interpretation module may compile information regarding sample collection and/or preparation, sample processing including nucleic acid sequencing, controls employed and control processing, metrics and visualizations of sequencing data and classification information, medical and diagnostic recommendations, practice recommendations, diagnostic reports, and any other useful information. An interpretation module may comprise an interface with which a user may interact, which interface may be common to other modules of a system of the present disclosure. The interface may comprise a web-based or locally-based portal that may be accessible by a user. Access to an interface of the present disclosure may be restricted to users having particular security clearance (e.g., in the interest of protecting patient privacy), by incorporation of passcodes and/or barcode scanning, etc. In some cases, different classes of users may be assigned different levels of access to an interface and modules with which it interacts. For example, a first class of users may have the ability to view patient information and diagnostic reports while a second class of users may be prohibited from viewing such information but may be able to access deidentified information about a sample and data visualizations. An interface of an interpretation module may include mechanisms for a user to input parameters for sequence analysis including, e.g., pathogens suspected of being included in a sample, other information about a sample, preferred controls, preferred analysis thresholds, reference databases for use in sequence analysis, etc. An interface of an interpretation module may also include mechanisms for a user to initiate repetition of an analytical process, optionally under refined conditions. An interface may comprise a portal via which a user may generate, update, download, upload, view; or otherwise interact with a report comprising a recommendation such as a therapeutic or medical recommendation, and/or a recommendation relating to, e.g., quarantine, site-specific processes including cleaning and disinfectant procedures, etc. (e.g., as described herein). A medical director or other professional may have access to and/or permission to generate such a report. An interpretation module may also be configured to provide a diagnostic report including metrics relating to sequencer performance and classification metrics and quality, which information may be stored within a database, laboratory information system, or customer relations management system. Such a database or system may be locally stored and/or may be stored within a web- or cloud-based system.

An interpretation module may comprise software with which a user may, e.g., visualize classification reports and metrics, among other features. Such software may comprise a variety of visualizations and textual data representations, which may be alterable based on user preference, downloaded or printed, uploaded to a server or other storage system, stored for later access, etc. Software may also comprise mechanisms for visualizing AMR genes and consensus sequencing results.

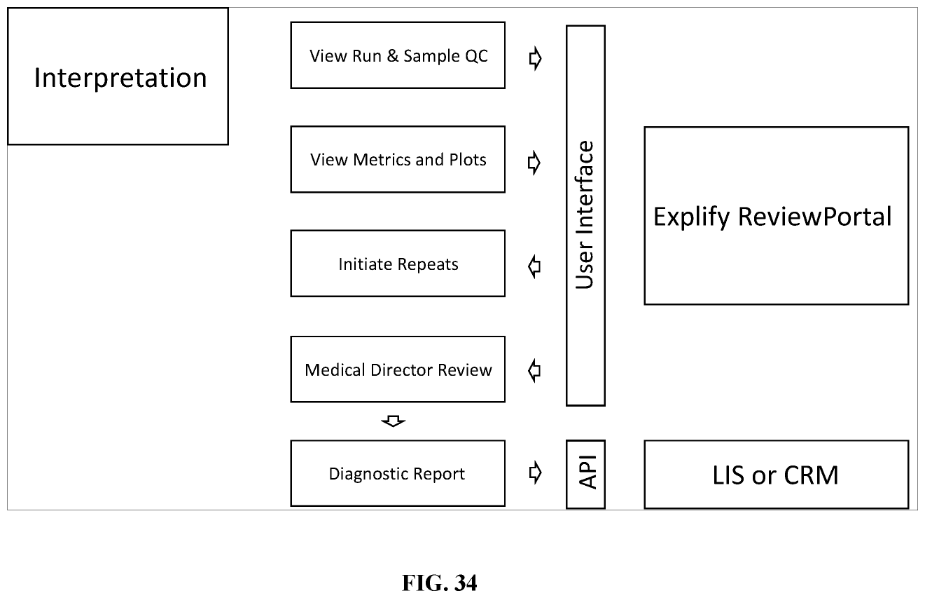

An example interpretation module is schematically illustrated in FIG. 34.

### Software.

The methods and systems provided herein may comprise the use of software to facilitate sample preparation, sample processing, sequencing, data collecting and processing, and/or data analysis, storage, and presentation. The present disclosure provides systems for providing information corresponding to a sample. A system for providing information corresponding to a sample may comprise a processor configured to display the information on a web-based graphical interface, where the information is represented by one or more visual and/or textual indicators (such as one or more graphs, bar charts, pie charts, scatter plots, 3D visualizations, text boxes, tables, or other indicators), including (i) an entity indicator, and (ii) a quality control indicator, where the information comprises the identities of one or more entities associated with the sample, where the entity indicator provides information about the identities of the one or more entities, and where the quality control indicator provides information about the certainty with which the identities of the one or more entities are determined. A method (e.g., a computer-implemented method) for providing information corresponding to a sample may comprise (a) providing data corresponding to the sample, where the data comprises a plurality of sequencing reads; (b) providing an interface to a user, where the interface displays to the user (i) an entity indicator (e.g., a visual and/or textual indicator) indicating that the plurality of sequencing reads correspond to one or more entities, and (ii) a quality control indicator (e.g., a visual and/or textual indicator) indicating the certainty with which the plurality of sequencing reads correspond to the one or more entities.

Entities corresponding to a sample may be, for example, a human and/or a microorganism. For example, an entity may be a human. In some cases, an entity may be a pathogen. An entity may be selected from the group consisting of a fungus, bacterium, parasite, and virus. In some cases, the one or more entities associated with a sample may comprise a first entity that is a human and a second entity selected from the group consisting of a fungus, bacterium, parasite, and virus. The second entity, and/or one or more other entities, may be associated with a disease or disorder, such as an infection. For example, the second entity may be associated with a disease or disorder, and/or the second entity and a third entity (e.g., another fungus, bacterium, parasite, or virus) may be associated with a disease or disorder. A sample may derive from a patient (e.g., a human patient). A patient from which a sample derives may have or be suspected of having a disease or disorder. In some cases, a patient from which a sample derives may have or be suspected of having a disease or disorder associated with a pathogen (e.g., bacteria, fungi, parasite, or virus). In some cases, a patient from which a sample derives may have been exposed or be suspected of having been exposed to a pathogen.

Information about a sample, such as information regarding entities associated with the sample, may be presented using a software program or platform. A software platform may comprise one or more components, such as a component for providing information about a sample, a component for analyzing sequencing information (e.g., performing a k-mer based analysis), a component for analyzing and classifying processed sequencing reads, and a component for supporting laboratory sample preparation. The software program is an example platform that includes three such features: a review portal (e.g., a web browser accessible dashboard application, an analysis pipeline that processes raw sequence data for analysis by a classification algorithm, and a sequence portal (e.g., web-based) application that supports sample information entry and laboratory sample preparation.

In some cases, information about a sample may be provided via a web-based interface. A web-based interface may be accessible using any web browser. A interface, whether it is web-based or not, may be accessible from a computing device, such as a personal or portable computing device or a stationary device. In some cases, the interface may be accessible from a computer disposed in a laboratory, hospital, clinic, or other setting. Certain features of the interface may be accessible without a network (e.g., internet) connection. For example, stored information about a previously analyzed sample may be accessible without a network connection. In some cases, information may be locally stored and accessible from the interface with or without a network connection.

An application in accordance with the present disclosure may comprise one or more sections that may be accessible from a main page or portal. The application may comprise a menu (e.g., a drop down menu, tabular menu, list, menu bar, or other menu) facilitating navigation between multiple sections. The menu may be accessible from some or all pages or sections of the application. For example, the menu may be accessible from the same location of each page or section. The one or more sections of an application in accordance with the present disclosure may include a main page or portal (e.g., a home page) from which a user may select to navigate to another section. For example, the main page or portal may comprise a log-in feature where a user may provide an assigned username and password to obtain access to the application. A user may select to view a particular report, such as a report associated with a given patient and/or sample. Report selection may be made, for example, in a section of the application accessible from a main page or portal.

A dashboard software application (e.g., accessible from a web browser) may enable detailed review of pathogens detected by a novel infectious disease diagnostic test based on, for example, methods and systems described elsewhere herein, specifically organism classification using metagenomics analysis software. Test results unique to methods and systems described elsewhere herein may be displayed for each suspected pathogen in an individual patient, in concert with quality control assessment of the underlying sequencing data (e.g., next generation sequencing) and controls.

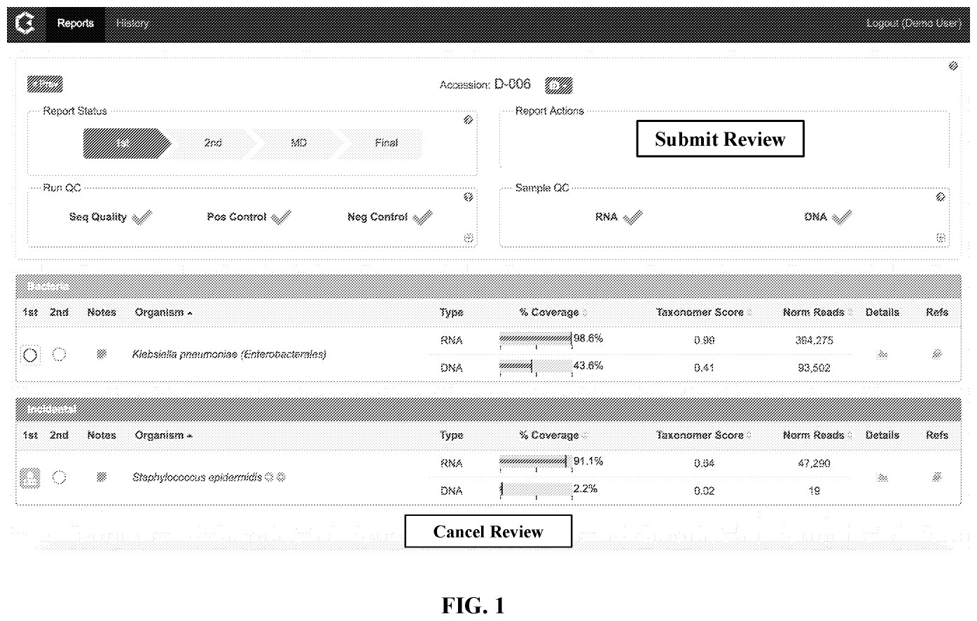

FIG. 1 displays an example interface for such an application. As shown in FIG. 1, the interface may comprise details of a report status (e.g., an indication of how many levels of review it has undergone by one or more scientists, technicians, medical professionals, doctors, or other reviews), assessments performed (e.g., quality control assessments), and entity identities. For instance, FIG. 1 indicates whether or not there has been first, second, medical doctor, and/or final review of the report. The report may also indicate whether both RNA and DNA sequencing reads have been analyzed. In the case of the report of FIG. 1, both RNA and DNA sequences have been read. Entity identities may be indicated graphically and/or textually. In some cases, an entity indicator may comprise a display corresponding to RNA analysis and a display corresponding to DNA analysis.

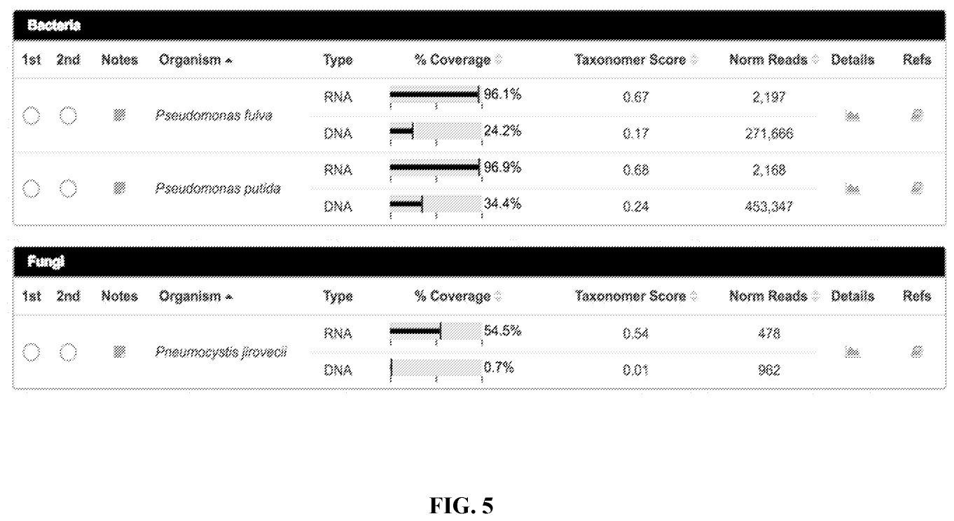

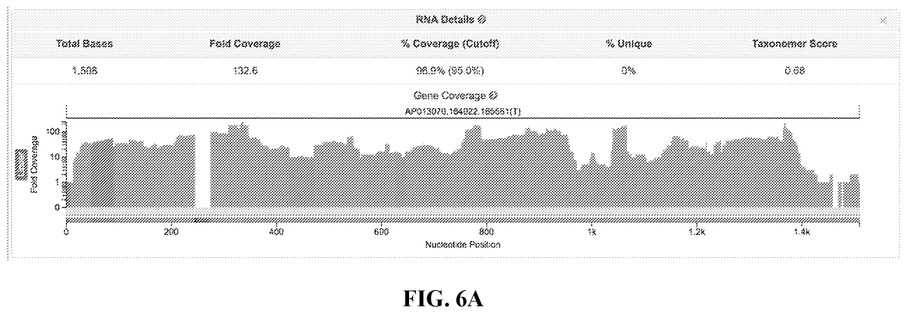

The methods and systems provided herein may facilitate identification of one or more entities (e.g., organisms) within a sample. FIG. 5 shows an example visualization for organism identification. As shown in FIG. 5, organisms may be grouped categorically (e.g., bacteria, fungi, and viruses).

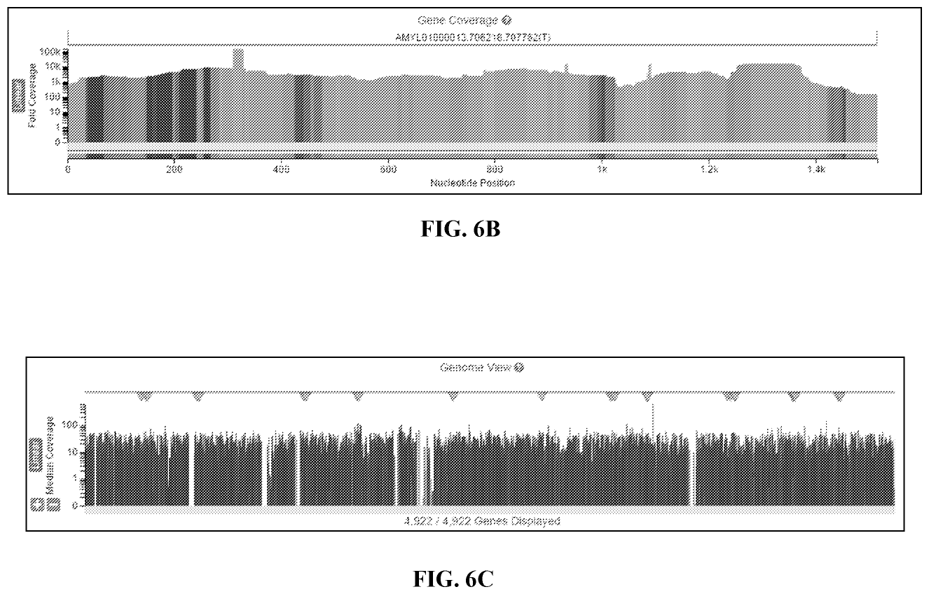

The results metrics of a diagnostic test, calculated from an organism classification algorithm, may be presented for each entity (such as each suspected pathogen) in a novel display, where sequencing read coverage is shown as bars along the genome or a gene, and the darker color of the bars represents the uniqueness of the regions of the reference genome or a gene. FIGS. 6A-6C show example visualizations for coverage at various nucleotide positions at the gene and genome levels. Results may be displayed based on k-mer analysis of sequencing read coverage, rather than sequencing reads. The total number of bases in a reference sequence, average number of estimated reads at each position along the reference sequence (fold coverage), minimum coverage required to display organism detection (% coverage), percentage of sequences unique to an organism as detected by a metagenomics analysis software (% unique), and/or a Score may also be provided. In some cases, a gene coverage plot such as that shown in FIG. 6B may display coverage depth at each base for the 16S/18S gene. A darker shade may signify a more unique portion of the gene, while gray areas may indicate less unique portions. The most unique portions may be highlighted by an additional indicator, such as a different color, texture, or pattern. The uniqueness indicated by such a gene coverage plot may be based on k-mer analysis (e.g., as described herein). In some cases, a genome view plot may be provided to allow visualization of an entire genome of an organism (FIG. 6C). The plot may display the median coverage depth for each gene. Genes with a higher total percent coverage may be indicated by, for example, a particular color, texture, or pattern.

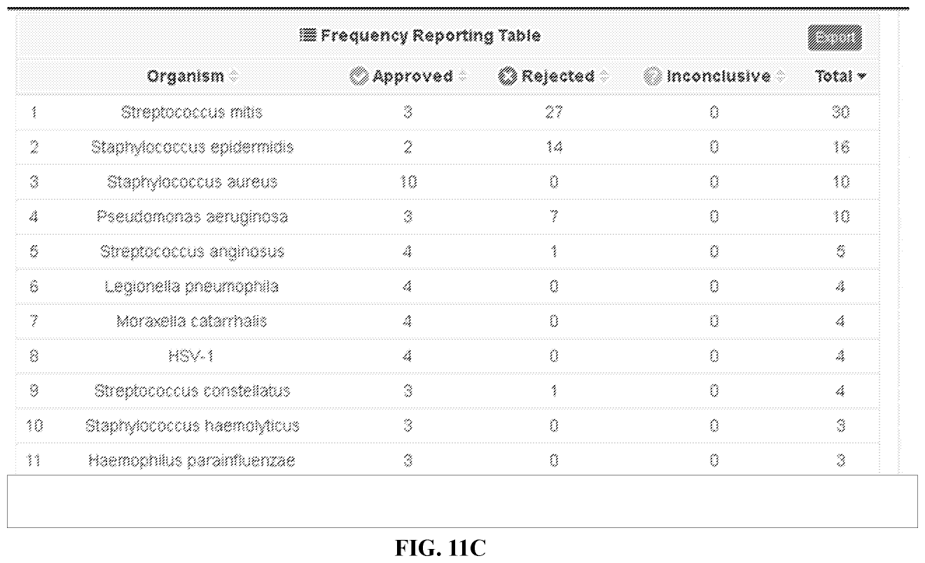

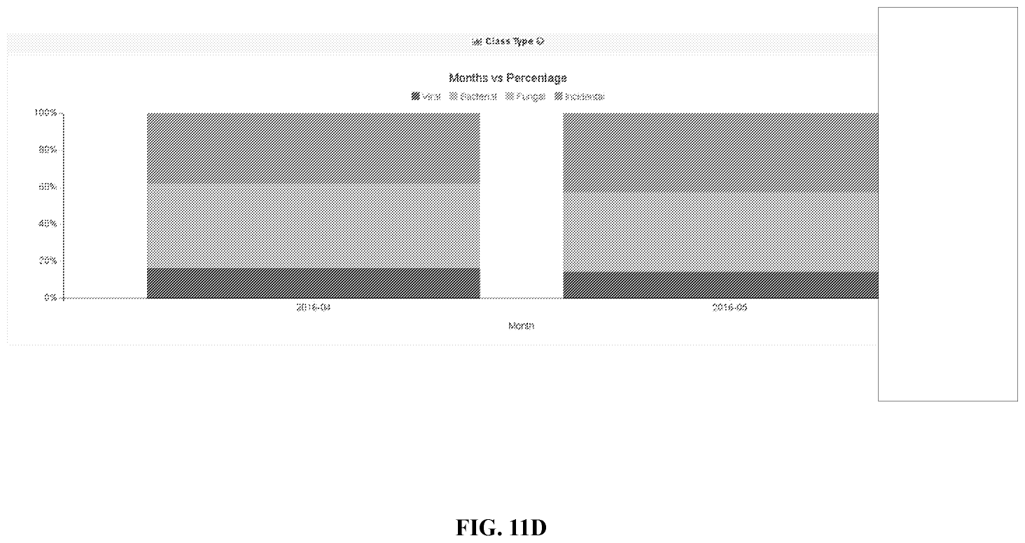

Results corresponding to sample information may be provided in a summary view. FIGS. 11A-11C show example visualizations including filters for selecting species of interest (FIG. 11A), a frequency chart for organisms (FIG. 11B), and a bar chart for organism types (FIG. 11C). These metrics may be provided in a separate section of an application (e.g., the web-based application) in accordance with the present disclosure.

The web-based application may also provide numerous quality control indicators for analyzing the quality of an analysis corresponding to a given sample. Different types of quality control indicators may be provided in different sections of the application in accordance with the present disclosure. Alternatively, all quality control indicators may be available in the same section of the application. In some cases, a user may choose to view or hide a given quality control metric, such as a visualization or other indicator. In some cases, the application may display pre-determined quality control metrics that may be selected by, for example, an administrator. In this case, quality control metrics may not be selectively filtered by any user but may only be changed by the administrator. The administrator may attain access to an editable version of the application by signing in to the application with an appropriate username and password.

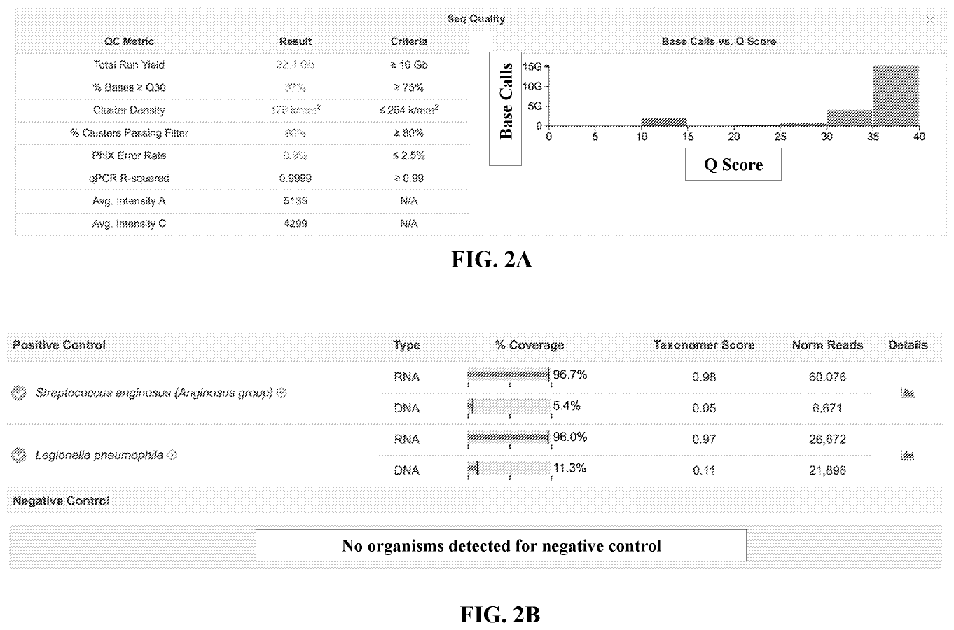

FIGS. 2A and 2B show example visualizations for sequencing quality control and processing control metrics, respectively. Quality metrics may include, for example, total run yield, cluster density, and other metrics and may be displayed alongside threshold metrics. Sequencing quality may also be indicated using a visualization displaying base calls relative to Q score, as shown in FIG. 2A. As shown in FIG. 2B, external processing controls (e.g., one or more positive or negative controls) may also be used to assess sequencing quality. The diagnostic test may use processing control samples that are run in parallel with patient samples, and a set of control organisms that may be added to all samples at the start of the laboratory sample preparation. The results from these external processing controls and internal control organisms are presented in novel ways in the context of assessing QC, estimating the level of test sensitivity, and reviewing individual suspected pathogens.

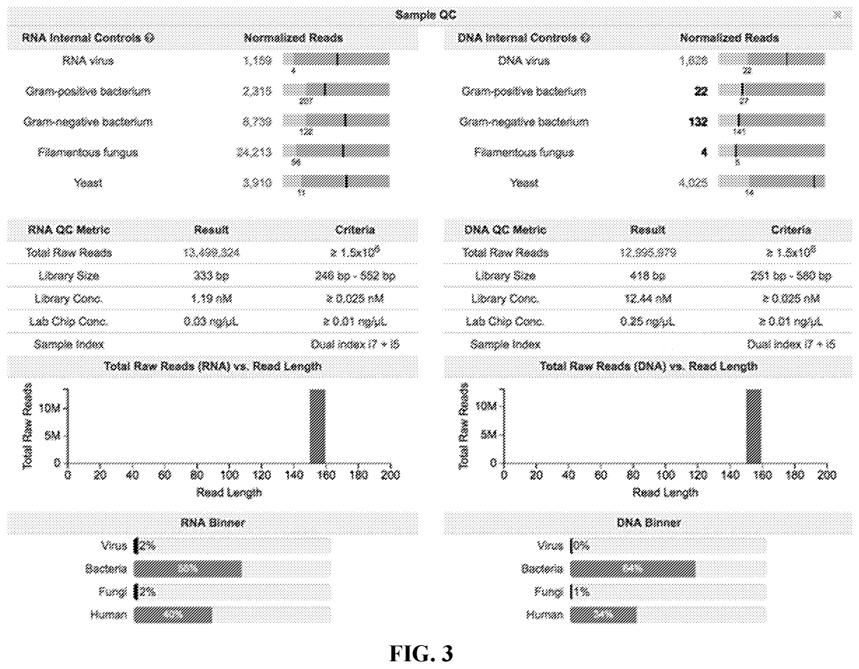

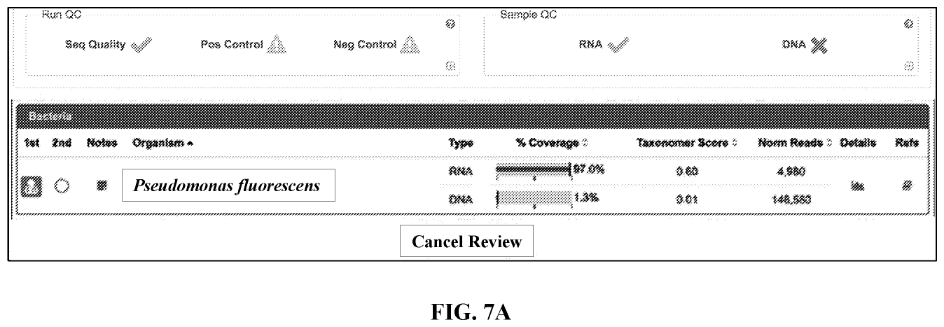

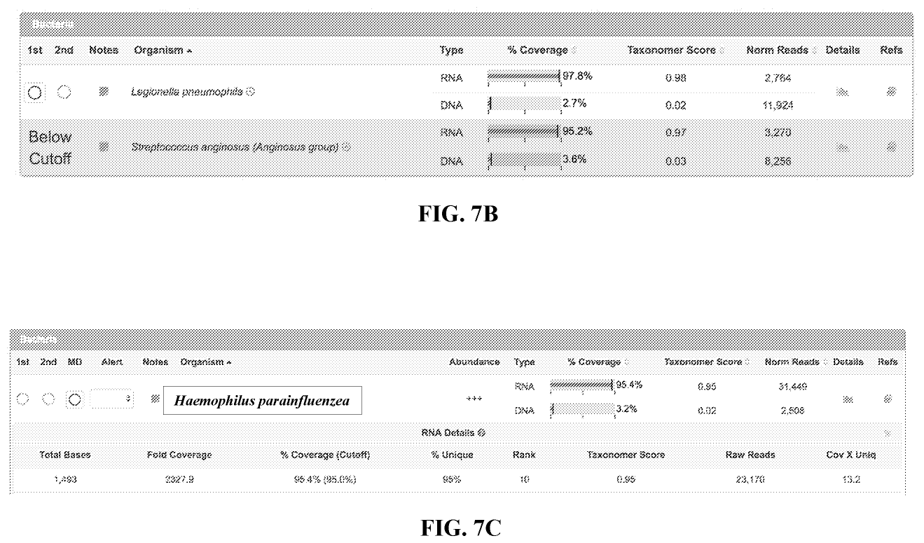

FIG. 3 shows another example visualization for sample quality control. Sample quality control metrics may be tracked for a given analysis (e.g., run) of a given sample. Sample quality control may be assessed separately for RNA and DNA. One or more indicators may be used to indicate that controls pass or do not pass a quality control check. FIGS. 7A-7C show example visualizations for quality control failure (FIG. 7A), organisms below cutoff in the positive processing control (FIG. 7B), and additional metrics for review (FIG. 7C).

The laboratory procedure creates sample libraries for sequencing, for the Illumina NGS platform, short double stranded adaptors are ligated to fragments of sample DNA. Combinations of adaptors containing different short index sequences may be randomly assigned to samples in a novel manner to mitigate contamination of data from previous sequencing runs. The application may provide a novel user interface to make manual changes to these assignments.

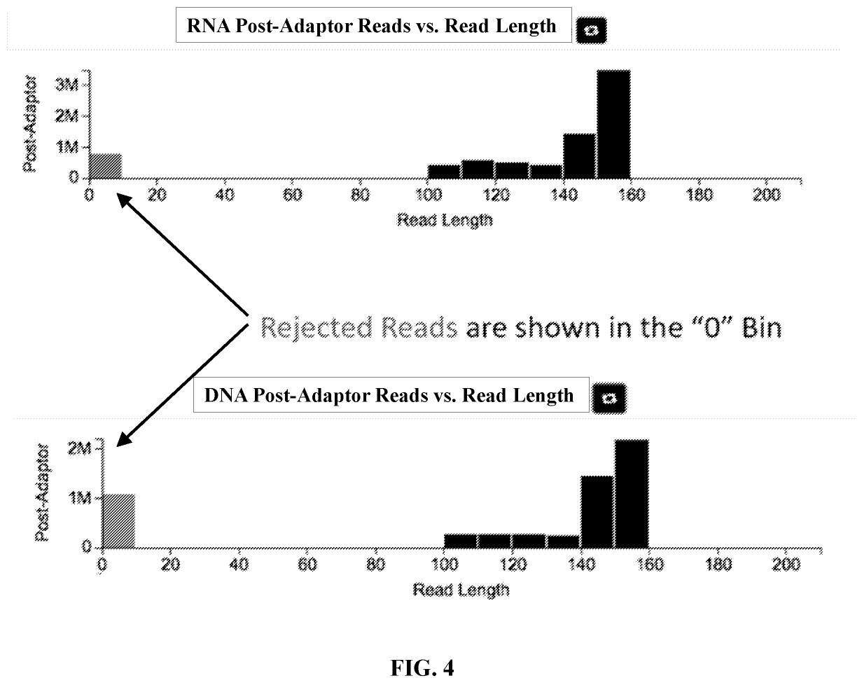

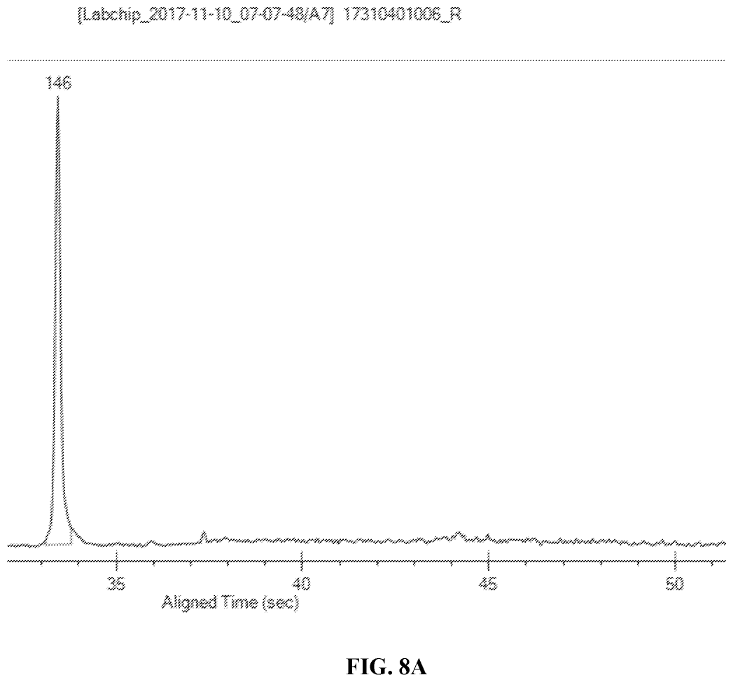

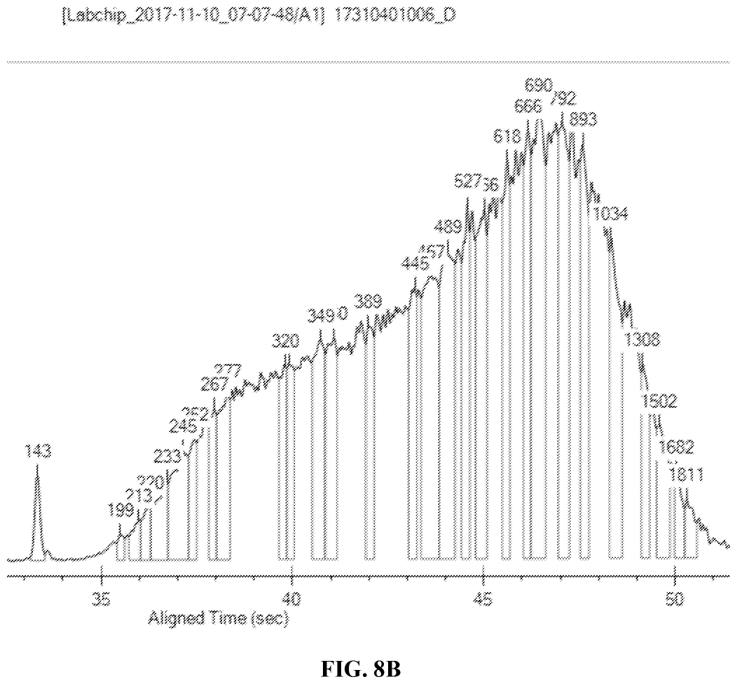

Adaptors can form non-informative dimers which are typically measured in the laboratory using electrophoresis methods. As part of quality control assessment, the occurrence of adaptor-dimers may be displayed in a novel view in the dashboard application and can serve as an in-silico alternative to electrophoresis (FIG. 4). Reads may be rejected if there are adapter sequences present. FIGS. 8A-8B show electrophoresis traces for quality control relating to adapter dimers. In FIGS. 8A-8B, the majority of rejected reads are due to adapter-dimers which appear in electrophoresis traces at around 145 base pairs.

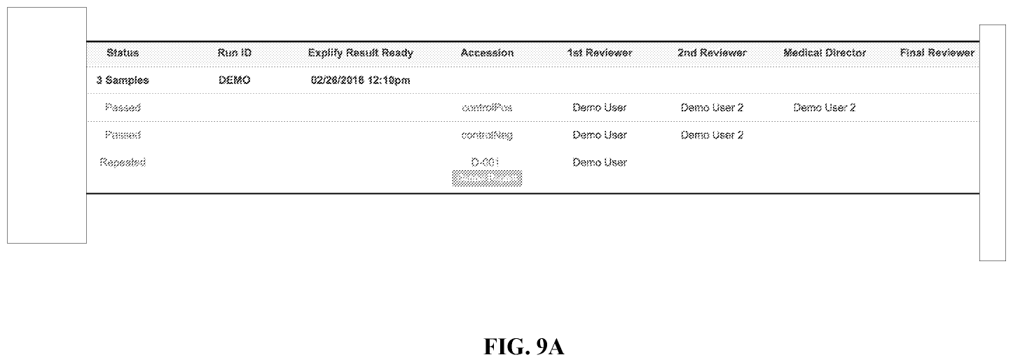

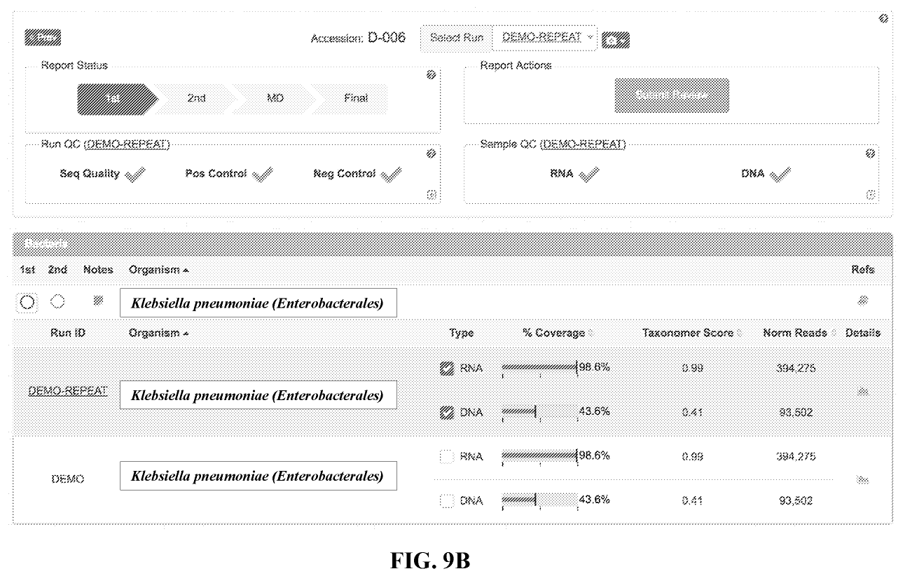

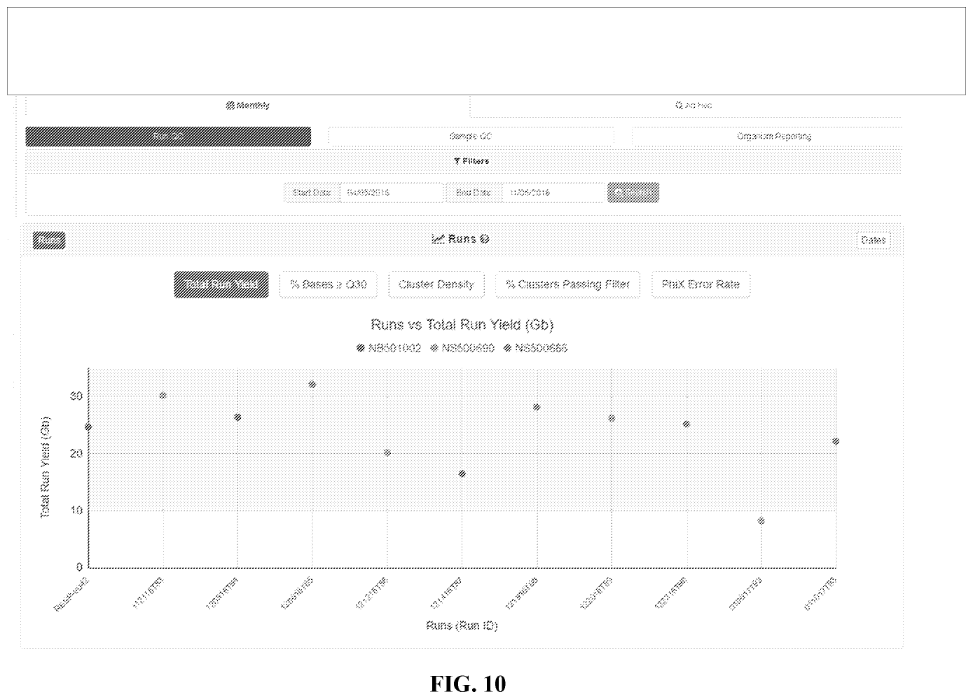

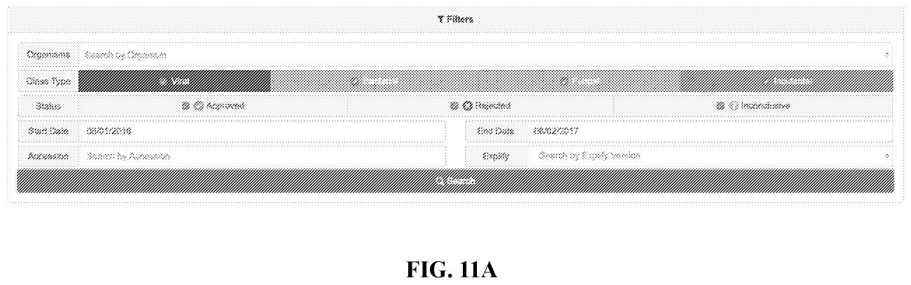

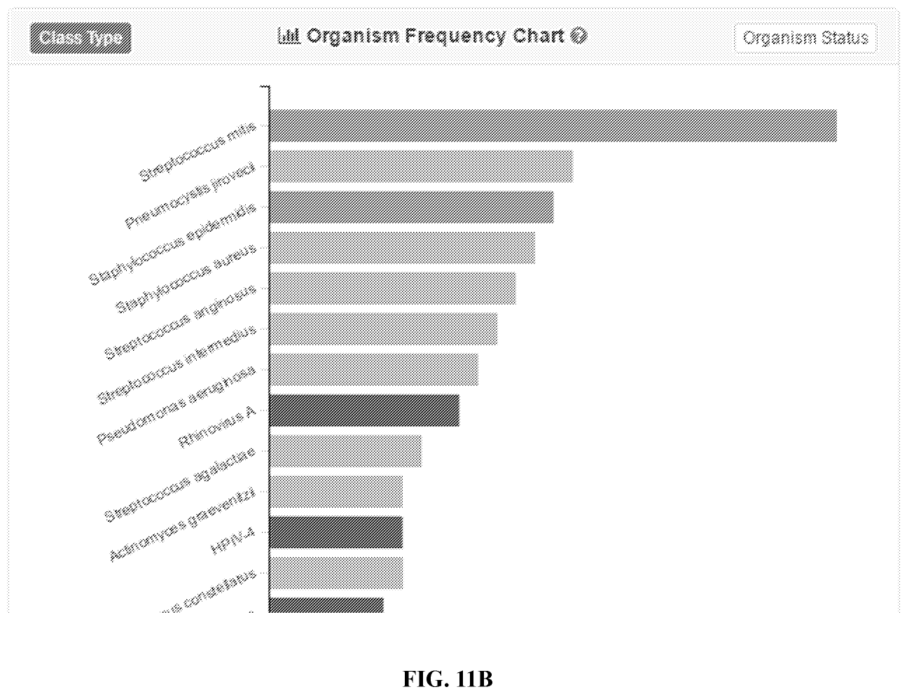

Occasionally a test may be repeated, resulting in more than one set of results for a given patient sample. The multiple sets of sequencing quality control data and analysis results may be presented in a novel way that allows a union view of the original set alongside newer sets from repeats. FIGS. 9A-9B show example visualizations corresponding to repeat runs, and FIG. 10 shows an example visualization for quality control metrics relating to repeated sequencing runs.

The dashboard application may support a workflow for, for example, diagnostic decision making. The workflow may involve multiple reviewers having different roles, such as technologist and medical director, through the novel use of visual elements that guide the review process and enforce workflow policies. For example, a report corresponding to a sample (e.g., a sample associated with a given patient) may be accessed through the interface by a technologist. The technologist may review the report and determine whether they agree with the report and/or believe that the data is of sufficient quality. They may enter their conclusions, as well as notes regarding their determination (e.g., whether another run should be performed, whether they draw any particular medical conclusion from the results, etc.), into an interface of the application. The report may also be analyzed by one or more additional users, including a doctor, clinician, or other medical professional.

The infectious disease diagnostic test can detect pathogens that of immediate public health concern. In some cases, a report may indicate that a sample is associated with one or more such pathogens. Accordingly, the application may use visual and/or textual cues for reporting Critical Alerts regarding public health pathogens. For example, the application may indicate that a pathogen of public health concern is present in a patient sample, and users may subsequently quarantine the patient or institute other protocols to prevent the pathogen from transferring to other persons or materials.

In some cases, the application in accordance with the present disclosure may provide a user with a diagnostic test profile. A diagnostic test profile may provide one or more properties associated with a subset of organisms within a scope of a diagnostic test. In some cases, the one or more properties comprises an organism name, an organism taxonomic rank, an organism class type, an organism sub-class, the organism membership in group based on phylogenetic and/or semantic relationship, medical relevance of an organism, validation, pathogen, RNA sensitive cutoff percentage, RNA specific cutoff percentage, DNA sensitive cutoff percentage, DNA specific cutoff percentage, highest scoring kmer, quantity of a particular kmer, or a combination thereof. In some cases, pathogen, organism taxonomic rank or organism class types may be as described elsewhere herein.

In some cases, medically relevant information is provided for organisms within a scope of a diagnostic test indicating whether such organisms are associated with any disease. In some cases, medically relevant may be whether an organism is mentioned within a publication. In some cases, medically relevant may be whether an organism name is within a publication. In some cases, medically relevant may be displayed on the diagnostic test profile. In some cases, medically relevant may be indicated by a flag (yes/no) based on a threshold of relevance. The threshold of relevance may be dependent on the number of publications that organism may be mentioned within.

In some cases, validation may refer to in-silico validation. In some cases, validation may refer to in-silico validation where sequences from known public sequence repositories may be added as simulated sequencing reads into background reads from sequencing non-pathogen containing (negative) samples.

In some cases, the diagnostic test profile may provide a user with a narrower scope of organisms as procured by the methods and systems described elsewhere herein. In some cases, the scope of organisms may be any organism. In some cases, the scope of organisms may be taken from the reference databases described elsewhere herein. In some cases, the user may expand the set of organisms. In some cases, the user may narrow the set of organisms. The user may expand the set of organisms to view unexpected organisms. The user may narrow the set of organisms to view more relevant organisms.

In some cases, the diagnostic test profile may display and/or calculate properties associated with a subset of organisms within the scope of organisms from the diagnostic test. The diagnostic test profile may display and/or calculate at least about 1, 2, 3, 4, 5, 6, 7, 8, 9, 10, 15, 20, 25, 30, 35, 40, 45, 50, 75, 100, 500, 1000, 5000, or more properties. The diagnostic test profile may display and/or calculate at most about 5000, 1000, 500, 100, 75, 50, 45, 40, 35, 30, 25, 20, 15, 10, 9, 8, 7, 6, 5, 4, 3, 2 or less properties. The diagnostic test profile may display and/or calculate 1 to 5000, 1 to 1000, 1 to 500, 1 to 50, 1 to 25, 1 to 10, 1 to 5, or 1 to 3 properties. In some cases, the properties may be selected by a user and/or computer. In some cases, the properties may be pre-selected by a user and/or computer.

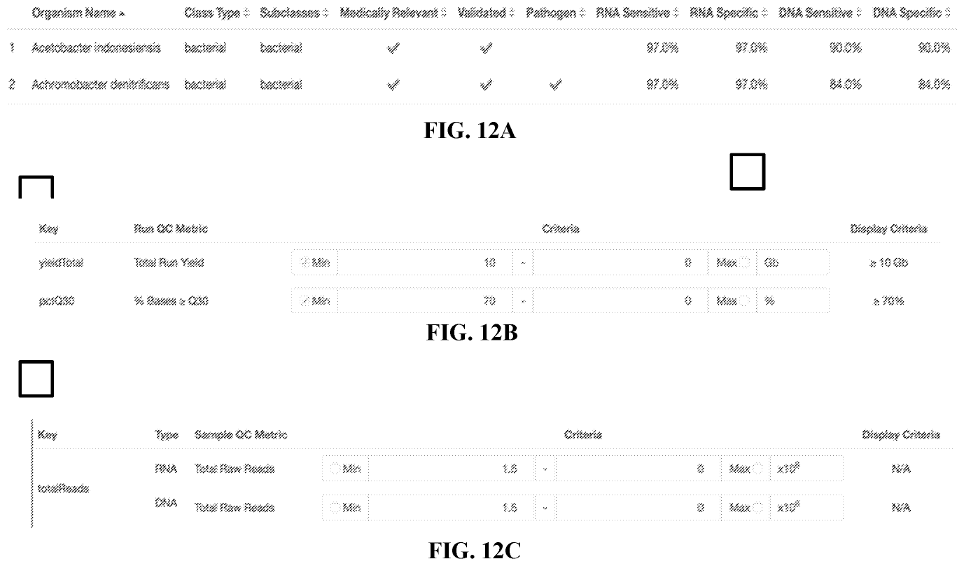

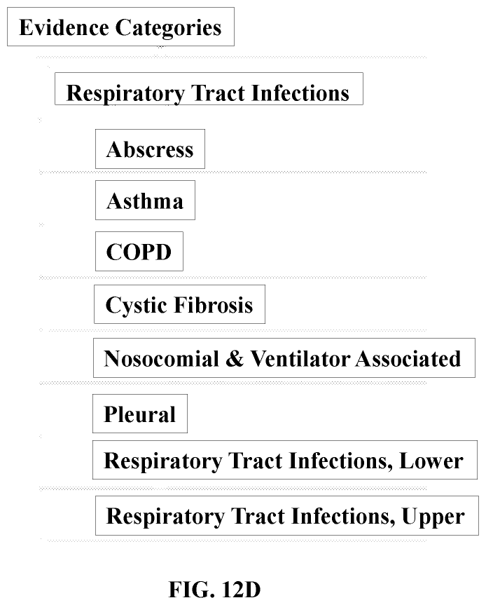

FIG. 12A shows an example visualization for the diagnostic test profile. The visualization shows an organism name, class type of the organism, subclasses of the organism, binary illustration of medically relevant (the check mark may indicate medically relevant, lack of a the check mark may indicate not validated), binary illustration validated (the check mark may indicate validated, lack of a check mark may indicate not validated), binary illustration of pathogen (the check mark may indicate medically relevant, lack of a the check mark may indicate not validated), RNA sensitive cutoff values, RNA specific cutoff values, DNA sensitive cutoff values, and DNA specific cutoff values. The visualization shows two rows of data pertaining to a diagnostic test profile. The visualization shows two rows of data with different organism names.

In some cases, the visualization may be displayed as a table with rows and columns. In some cases, the visualization may be displayed as a list, graph, chart, Venn diagram, or numeric indicators, etc. In some cases, the visualization may be adjusted by the user or a computer. In some cases, the visualization may be adjusted to a specific format tailored to the desire or need of a user.

In some cases, the properties displayed by the visualization may be, for example, organism names, organism taxonomic ranks, organism class types, organism sub-class types, pathogens, RNA sensitive cutoff percentage, RNA specific cutoff percentage, DNA sensitive cutoff percentage, DNA specific cutoff percentage, medically relevant, and validated, etc. In some cases, the diagnostic test profile may have at least about 1, 2, 3, 4, 5, 6, 7, 8, 9, 10, 15, 20, 30, 40, 50, 100, 500, 1000 or more rows of data pertaining to a diagnostic test profile. In some cases, the diagnostic test profile may have at most about 1000, 500, 100, 50, 40, 30, 20, 15, 10, 9, 8, 7, 6, 5, 4, 3, 2, or less rows of data pertaining to a diagnostic test profile. In some cases, the diagnostic test profile may have from about 1 to 1000, 1 to 100, 1 to 50, 1 to 10, or 1 to 5 rows of data pertaining to a diagnostic test profile.

In some cases, the RNA sensitive cutoff percentage displayed and/or selected may be at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more. In some cases, the RNA sensitive cutoff percentage may be at most about 100%, 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50% or less. In some cases, the RNA sensitive cutoff percentages may be from about 50% to 100%, 60% to 100%, 70% to 100%, 80% to 100%, 85% to 100%, 90% to 100%, or 95% to 100%.

In some cases, the RNA specific cutoff percentage displayed and/or selected may be at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more. In some cases, the RNA specific cutoff percentage may be at most about 100%, 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50% or less. In some cases, the RNA specific cutoff percentage may be from about 50% to 100%, 60% to 100%, 70% to 100%, 80% to 100%, 85% to 100%, 90% to 100%, or 95% to 100%.

In some cases, the DNA sensitive cutoff percentage displayed and/or selected may be at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more. In some cases, the DNA sensitive cutoff percentage may be at most about 100%, 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50% or less. In some cases, the DNA sensitive cutoff percentage may be from about 50% to 100%, 60% to 100%, 70% to 100%, 80% to 100%, 85% to 100%, 90% to 100%, or 95% to 100%.

In some cases, the DNA specific cutoff percentage displayed and/or selected may be at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more. In some cases, the DNA specific cutoff percentage may be at most about 100%, 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50% or less. In some cases, the DNA specific cutoff percentage may be from about 50% to 100%, 60% to 100%, 70% to 100%, 80% to 100%, 85% to 100%, 90% to 100%, or 95% to 100%.

In some cases, the diagnostic test profile may display and/or calculate the run-level quality control criteria for the diagnostic test. FIG. 12B shows an example visualization for the run-level quality control. The run-level quality control visualization shows a key, run quality control metric, criteria, display criteria, yield total, percentage of Q30, percentages of bases with greater than Q30, display criteria percentages, and display criteria data size. The run-level quality control visualization shows two rows of data pertaining to the run-level quality control information. The run-level quality control visualization shows that the criteria has a minimum that may be selected or unselected. The run-level quality control visualization shows that the criteria has a maximum that may be selected or unselected. The run-level quality control visualization shows that the criteria has values that a user or computer may input or adjust.

In some cases, the run-level quality control visualization may have at least about 1, 2, 3, 4, 5, 6, 7, 8, 9, 10, 15, 20, 30, 40, 50, 100, 500, 1000 or more rows (or records) of data pertaining to 1, 2, 3, 4, 5, 6, 7, 8, 9, 10, 15, 20, 30, 40, 50, 100, 500, 1000 or more criteria of the run-level quality control. In some cases, the run-level quality control visualization may have at most about 1000, 500, 100, 50, 40, 30, 20, 15, 10, 9, 8, 7, 6, 5, 4, 3, 2, or less rows (or records) of data pertaining to at most 1000, 500, 100, 50, 40, 30, 20, 15, 10, 9, 8, 7, 6, 5, 4, 3, 2, or few criteria of the run-level quality control. In some cases, the run-level quality control visualization may have from about 1 to 1000, 1 to 100, 1 to 50, 1 to 10, or 1 to 5 rows of data pertaining to the run-level quality control.

In some cases, the run-level quality control visualization may be displayed as a table with rows and columns. In some cases, the run-level quality control visualization may be displayed as a list, graph, chart, venn diagram, or numeric indicators, etc. In some cases, the run-level quality control visualization may be adjusted by the user or a computer. In some cases, the run-level quality control visualization may be adjusted to a specific format tailored to the desire or need of a user.

In some cases, the run-level metrics may be, for example, total yield, total run yield, yield perfect, percentage of bases greater than or equal to Q30 (% Q>=30), cluster density, percentage of clusters passing filter, PhiX error rate, percentage of tile pass, intensity of A, intensity of C, projected total yield, yield <=n errors, or any combination thereof, etc.

In some cases, total yield may be the number of bases sequenced. In some cases, the total yield may be updated as the run progresses.

In some cases, total run yield may be the number of bases sequenced. In some cases, total run yield may be the number of bases sequenced which passed filter.

In some cases, yield perfect may be the number of bases in reads that align perfectly. In some cases, yield perfect may be the number of baes in reads that align perfectly as determined by alignment to PhiX of reads derived from a spiked in PhiX control sample. In some cases, if a PhiX control sample is not run in the lane, this chart may not be available.

In some cases, % Q>=30 may be the percentage of bases with a quality score of 30 or higher. In some cases, the chart may be generated after the N^{th} cycle, where N is a positive integer between 10 and 200 (e.g., N=25 would mean the 25^{th} cycle). In some cases, the values represent the current cycle.

In some cases, cluster density may be the density of clusters (in thousands per mm²) detected by image analysis. In some cases, cluster density may be the density of clusters (in thousands per mm²) detected by image analysis, +/− one standard deviation.

In some cases, percentage of clusters passing filter may be the percentage of clusters passing filtering, +/− one standard deviation.

In some cases, PhiX error rate may be the calculated error rate, as determined by a spiked in PhiX control sample.

In some cases, percentage of tile pass may be the percentage of tiles that have a passing value. In some cases, the tile may indicate the progress of base calling. In some cases, the tile may indicate the quality scoring.

In some cases, intensity of A may be the average of the A channel intensity measured at the first cycle averaged over filtered clusters. In some cases, intensity of A may be the A channel intensity.

In some cases, intensity of C may be the average of the C channel intensity measured at the first cycle averaged over filtered clusters. In some cases, intensity of C may be the C channel intensity.

In some cases, projected total yield may be the projected number of bases expected to be sequenced at the end of the run.

In some cases, yield <=n errors may be the number of bases in reads that align with n errors or less, as determined by a spiked in PhiX control sample. N may be any integer, for example, 1, 2, 3, 4, 5, 6, 7, 8, 9, 10, etc.

In some cases, the diagnostic test profile may display and/or calculate the sample-level quality control criteria for the diagnostic test. FIG. 12C shows an example visualization for the sample-level quality control. The sample-level quality control visualization shows a key, type, sample quality control metric, criteria, display criteria, total reads, RNA type, DNA type, and total raw reads. The sample-level quality control visualization shows two rows of data pertaining to the run-level quality control information. The sample-level quality control visualization shows that the criteria has a minimum that may be selected or unselected. The sample-level quality control visualization shows that the criteria has a maximum that may be selected or unselected. The sample-level quality control visualization shows that the criteria has values that a user or computer may input or adjust.

In some cases, the sample-level quality control visualization may have at least about 1, 2, 3, 4, 5, 6, 7, 8, 9, 10, 15, 20, 30, 40, 50, 100, 500, 1000 or more rows (or records) of data pertaining to 1, 2, 3, 4, 5, 6, 7, 8, 9, 10, 15, 20, 30, 40, 50, 100, 500, 1000 criteria of the sample-level quality control. In some cases, the sample-level quality control visualization may have at most about 1000, 500, 100, 50, 40, 30, 20, 15, 10, 9, 8, 7, 6, 5, 4, 3, 2, or less rows or records of data pertaining to 1000, 500, 100, 50, 40, 30, 20, 15, 10, 9, 8, 7, 6, 5, 4, 3, 2, or fewer criteria the sample-level quality control. In some cases, the sample-level quality control visualization may have from about 1 to 1000, 1 to 100, 1 to 50, 1 to 10, or 1 to 5 rows or records of data pertaining to from about 1 to 1000, 1 to 100, 1 to 50, 1 to 10, or 1 to 5 criteria of the sample-level quality control.

In some cases, the run-level quality control visualization may be displayed as a table with rows and columns. In some cases, the run-level quality control visualization may be displayed as a list, graph, chart, Venn diagram, or numeric indicators, etc. In some cases, the run-level quality control visualization may be adjusted by the user or a computer. In some cases, the run-level quality control visualization may be adjusted to a specific format tailored to the desire or need of a user.

In some cases, the sample-level metrics may be, for example, total raw reads, unique reads, post-adaptor reads, post-quality reads, total IC norm reads, entropy, G content, library Q score, library size, library concentration, etc.

In some cases, raw reads may be the reads in a file. In some cases, raw reads may be reads in a demultiplexed Fastq file.

In some cases, unique reads may be unique reads in a file. In some cases, unique reads may be unique reads in a demultiplexed Fastq file.

In some cases, post-adaptor reads may be reads after adaptor trimming in a file. In some cases, post-adaptor reads may be reads after adaptor trimming of a demultiplexed Fastq file.

In some cases, post-quality reads may be reads after applying a quality filter and trimming. In some cases, post-quality reads may be reads after applying a quality filter. In some cases, post-quality reads may be reads after applying trimming.

In some cases, total IC norm reads may be normalized read count of internal control organism(s).

In some cases, entropy may be the Shannon Diversity index of sequence complexity in the post-quality Fastq.

In some cases, library Q score may be the Phred scaled quality score of base calls in the post-quality Fastq.

In some cases, library size may be the estimate library size based on electrophoresis. In some cases, library size may be the estimate library size based on electrophoresis in the lab.

In some cases, library concentration may be the estimated library concentration based on qPCR or other methods. In some cases, library concentration may be the estimated library concentration based on qPCR in the lab.

In some cases, the properties, run-level criteria, and/or sample-level criteria may be tuned by a user through a graphical interface as shown in FIGS. 12A-12C. In some cases, the properties, run-level criteria, and/or sample-level criteria may be tuned by a computer and/or a user. In some cases, the amount of properties, run-level criteria, and/or sample-level criteria displayed may be reduced. In some cases, the amount of properties, run-level criteria, and/or sample-level criteria may be increased.

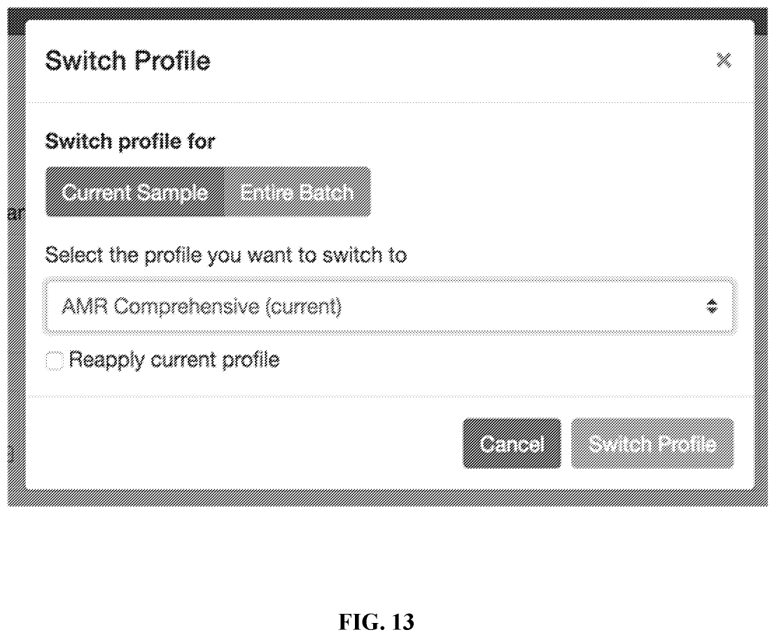

In some cases, a user may change the diagnostic test profile that is displayed. A user may change a diagnostic test profile to expand the set of organisms to look for unexpected organisms or to narrow the set for more relevant organisms. FIG. 13 shows an example visualization for switching diagnostic test profiles. The switching diagnostic test profile visualization shows different batches that have different names. The switching diagnostic test profile visualization has a drop-down menu that a user can use to switch profiles. The switching diagnostic test visualization has an option to cancel switching profiles as well as the option to switch profiles. The switching diagnostic test visualization has the option to reapply the current profile.

In some cases, the user may view more than a single diagnostic test profile. In some cases, the user may view at least about 1, 2, 3, 4, 5, 6, 7, 8, 9, 10, 15, 20, 25, 30, 35, 40, 45, 50, 100, 500, 1000 or more diagnostic test profiles. In some cases, the user may view at most about 1000, 500, 100, 50, 45, 40, 35, 30, 25, 20, 15, 10, 9, 8, 7, 6, 5, 4, 3, 2 or less diagnostic test profiles. In some cases, the user may view about 1 to 1000, 1 to 100, 1 to 50, 1 to 10, or 1 to 5 diagnostic profiles. In some cases, the user may combine diagnostic test profiles. In some cases, the user may generate a report of one or more diagnostic test profiles. In some cases, the user may save a diagnostic test profile. In some cases, the user may give a diagnostic test profile a name. In some cases, the name of a diagnostic test profile may be randomly generated. In some cases, the diagnostic test profile may be used as a template for a different diagnostic template. In some cases, the user may select a different profile using, for example, a drop-down menu of profiles, a list of profiles, or a row of profiles, etc. The user may have at least about 1, 2, 3, 4, 5, 6, 7, 8, 9, 10, 11, 12, 13, 14, 15, 20, 30, 40, 50, 100, 500, 1000 or more saved diagnostic test profiles. The user may have at most about 1000, 500, 100, 50, 40, 30, 20, 15, 14, 13, 12, 11, 10, 9, 8, 7, 6, 5, 4, 3, 2, or less saved diagnostic test profiles. The user may have from about 1 to 1000, 1 to 100, 1 to 10, or 1 to 5 saved diagnostic test profiles.

In some cases, the diagnostic test profile may apply a disease category. The disease category may limit the scope of diagnostic test results. In some cases, the user may further limit the scope by selecting a disease sub-category as shown in FIG. 12D. The visualization shown in FIG. 12D displays a disease category. The visualization shows sub-categories of the disease. The disease category and disease sub-categories are shown in a drop-down menu and can be selected by a user. A disease category may be any disease, for example, respiratory tract infection. A disease sub-category may be any disease. A disease sub-category may be any disease that is within the scope of a larger disease category, for example, asthma falls under the scope of respiratory tract infections. In some cases, a user may define their own disease categories and/or disease sub-categories. In some cases, the disease category may be given a name. In some cases, the user may select the disease and/or disease sub-category using, for example, a drop-down menu, graph, search box, list, or chart, etc.

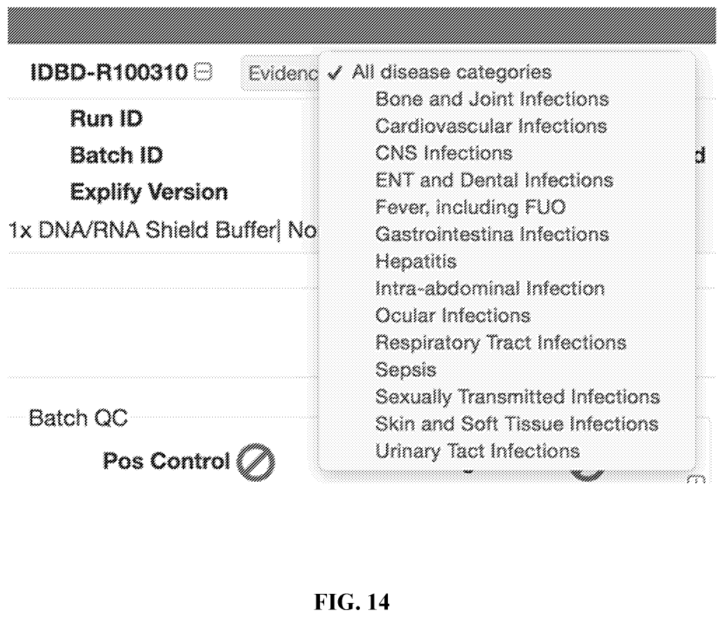

In some cases, an application in accordance with the present disclosure may provide more information of the organisms. The application may provide a user with a collection of information. In some cases, the collection of information may be displayed on a diagnostic test profile. The collection of information may be, for example, publications (e.g. scientific publications, news publications, etc). The publications may associate an organism with disease categories. The disease categories may be any disease. The disease categories may be, for example, bone and join infections, cardiovascular infections, central nervous system (CNS) infections, enteric nervous system (ENT) and dental infections, fever including fevers of unknown origin (FUO), gastrointestinal infections, hepatitis, intra-abdominal infection, ocular infections, etc. FIG. 14 shows an example visualization that may allow a user to select a disease category using a graphical user interface. The visualization shows a drop-down menu with the disease categories that a user can select. The selection of a disease category can narrow the search results to organisms that pertain to that disease category. The visualization also displays the run identification and the batch identification numbers of the diagnostic test. The visualization also shows the current version of software. The visualization can show one or more disease or disease sub-categories. The user may narrow the disease or disease sub-categories so that a selection can be viewed. In some cases, the user may select the disease and/or disease sub-category using, for example, a drop-down menu, graph, search box, list, or chart, etc. The visualization can show any other information to a user.

In some cases, the collection of information may be categorized by a user and/or computer. The collection of information may be categorized by a natural language processing system. The natural language processing system may be trained by a user and/or computer. The natural language processing system may have a user and/or computer set parameters. The parameters may be, for example syntax, semantics, discourse, or speech style, etc. The collection of information may be categorized on certain keywords found in the publications, potential pathogens associated with a disease, a user's understanding of the field, etc. The natural language processing system may be updated at any time. In some cases, the collection of information may be given a name, for example, evidence.

In some cases, when a category is selected by the user, the collection of information may be presented by an external source outside the web-based application. In some cases, the collection of information may be presented to the user within the web-based application. In some cases, the collection of information may be from a web search engine, for example, Google, Bing, or Yahoo, etc. In some cases, the collection of information may be from a database, for example, NCBI PubMed, PubMed, Scifinder, or Google Scholar, etc. In some cases, the database and/or web search engine may present to a user a list of publications.

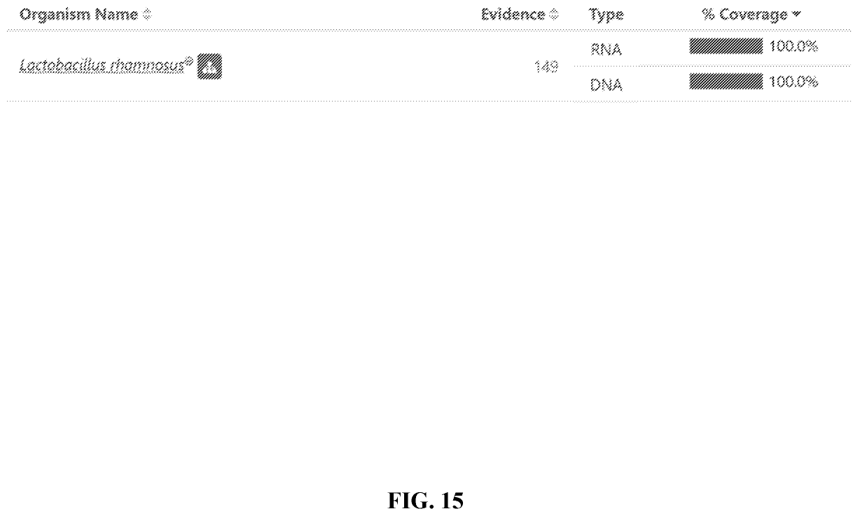

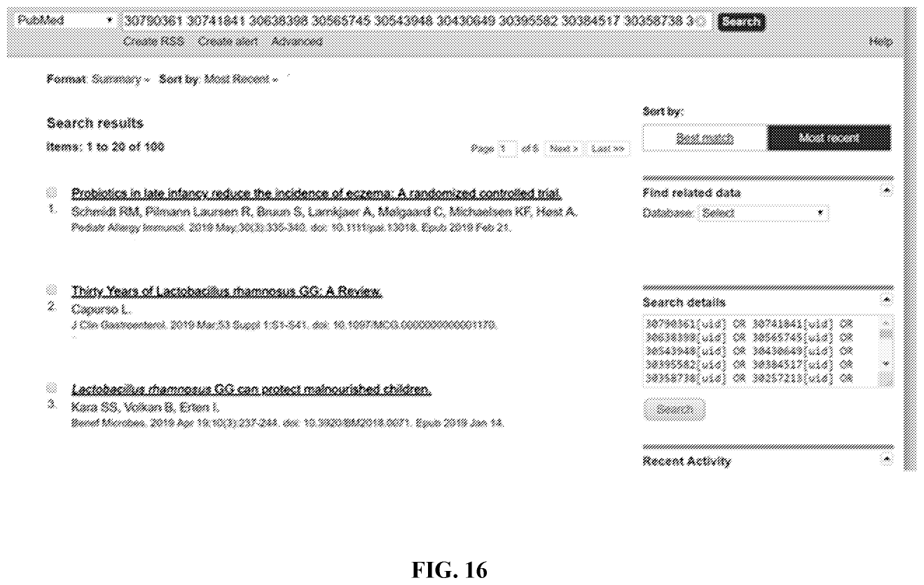

In some cases, one or more publications may be displayed on the diagnostic test profile as shown in FIG. 15. In FIG. 15, the visualization shows the organism name, Lacobacillus rhamnosus next to a clickable icon that can link a user to the phylogenetic tree. In addition, the visualization shows the number of publications (e.g., 149) that pertain to the organism name. The visualization also shows the type and percentage coverage. Here the percentage coverage is the percentage of the genome of the identified species that was found in the test sample (e.g., first sample). The percentage coverage has a numerical and color indicator. The number of publications may be an indirect measurement of relevance. In some cases, the organisms may be sorted by the number of publications. In some cases, the number of publications may be a hyperlink that may send a user to a webpage and/or database that may display each publication to the user, as shown in FIG. 16. As shown in FIG. 16, a list of publications that pertain to the Lactobacillus rhamnosus are displayed. When the user clicks on the number of publications, the user is sent to an external website. The publications are displayed by PubMed website. The selection of publications displayed have been procured beforehand. The selection of publications may be procured by a user or computer. The selection of publications may be procured on relevance. Relevance may have a variety of criteria that a user or computer may define beforehand or after.

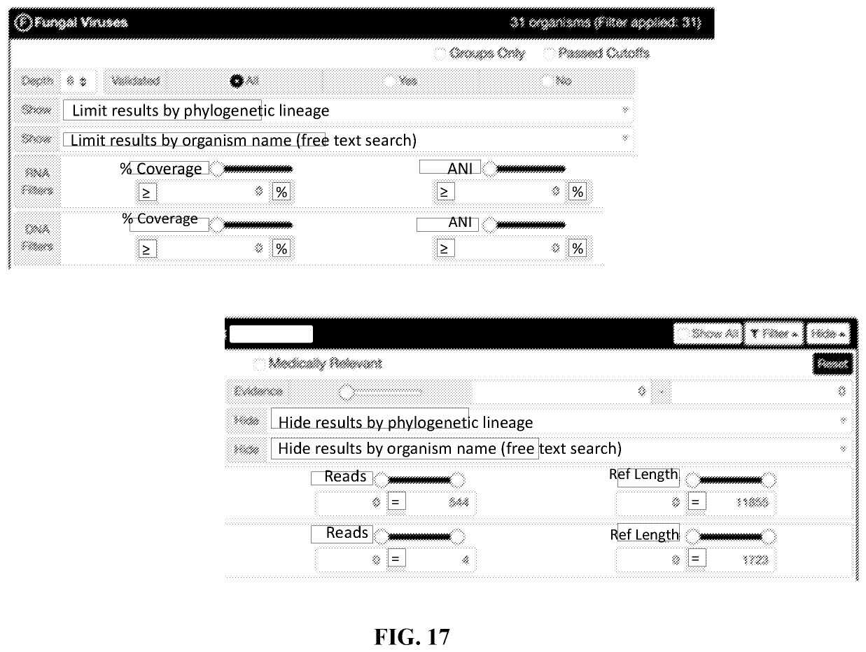

In some cases, the user may apply a filter to the diagnostic test profile. The user may apply a filter to refine or expand the set of detected organisms. The user may apply a filter to avoid false negative results. FIG. 17 shows an example visualization of a filter interface that a user may use. The filter interface visualization shows a variety of filters that a user can use to expand or narrow the results from the diagnostic test.

For example, the filter interface visualization shows that a user can limit/expand by the percentage coverage using the slider icon or inputting a value of the Percent coverage (RNA) filter. That is by inputting a numerical value between 0 and 100 percent, the user can specify that, in order for a corresponding species to be identified in the sample, at least that specified percentage of the total RNA for that species must be present.

Also, for example, the filter interface visualization shows that a user can limit/expand by the average RNA identity using the slider icon or inputting a value of the ANI (RNA) filter.

Also, for example, the filter interface visualization shows that a user can limit/expand by number of reads using the slider icon or inputting a value of the Read (RNA) filter. That is by inputting a numerical value for the Read (RNA) filter, the user can specify that, in order for a corresponding species to be identified in the sample, at least that number of reads must be present in the test sample.

Also for example, the filter interface visualization shows that a user can limit/expand by the reference length using the slider icon or inputting a value of the Ref Length (RNA) filter. That is by inputting a numerical value for the Ref Length (RNA) filter, the user can specify that, in order for reference sequence in a set of reference sequences to be used in the comparisons it must have the length specified.

The filter interface visualization shows that a user can limit/expand the corresponding parameters for DNA as well. For example the user can limit/expand by the percentage coverage using the slider icon or inputting a value of the Percent coverage (DNA) filter, limit/expand by the average nucleotide identity using the slider icon or inputting a value of the ANI (DNA) filter, limit/expand by the reads using the slider icon or inputting a value of the Reads (DNA) filter, and/or limit/expand by the reference length using the slider icon or inputting a value of the Reference Length (DNA) filter.

The filter interface visualization also shows that a user can limit/expand results by phylogenetic lineage, limit/expand results by organism name by free text search, hide results by phylogenetic lineage, hide results by organism name using free text search, limit/expand by the quantity of evidence.

In some cases, the RNA filter coverage percentage coverage may be at least about 0%, 5%, 10%, 15%, 20%, 25%, 30%, 35%, 40%, 45%, 50%, 55%, 60%, 65%, 70%, 75%, 80%, 85%, 90%, 95%, 99% or more. In some cases, the RNA filter coverage percentage may be at most about 99%, 95%, 90%, 85%, 80%, 75%, 70%, 65%, 60%, 55%, 50%, 45%, 40%, 35%, 30%, 25%, 20%, 15%, 10%, 5%, or less. In come cases, the RNA filter coverage percentage coverage may be from about 0% to 100%, 0% to 95%, 0% to 90%, 0% to 85%, 0% to 80%, 0% to 75%, 0% to 70%, 0% to 65%, 0% to 60%, 0% to 55%, 0% to 50%, 0% to 45%, 0% to 40%, 0% to 35%, 0% to 30%, 0% to 25%, 0% to 20%, 0% to 15%, 0% to 10%, or 0% to 5%.

In some cases, the RNA filter average nucleotide identity may be at least about 0%, 5%, 10%, 15%, 20%, 25%, 30%, 35%, 40%, 45%, 50%, 55%, 60%, 65%, 70%, 75%, 80%, 85%, 90%, 95%, 99% or more. In some cases, the RNA filter average nucleotide identity may be at most about 99%, 95%, 90%, 85%, 80%, 75%, 70%, 65%, 60%, 55%, 50%, 45%, 40%, 35%, 30%, 25%, 20%, 15%, 10%, 5%, or less. In some cases, the RNA filter average nucleotide identity may be from about 0% to 100%, 0% to 95%, 0% to 90%, 0% to 85%, 0% to 80%, 0% to 75%, 0% to 70%, 0% to 65%, 0% to 60%, 0% to 55%, 0% to 50%, 0% to 45%, 0% to 40%, 0% to 35%, 0% to 30%, 0% to 25%, 0% to 20%, 0% to 15%, 0% to 10%, or 0% to 5%.

In some cases, the RNA filter reads may be at least about 0, 5, 10, 15, 30, 50, 100, 200, 300, 400, 500, 600, 700, 800, 900, 1000, 10000 or more. In some cases, the RNA filter reads may be at most about 10000, 1000, 900, 800, 700, 600, 500, 400, 300, 200, 100, 50, 30, 15, 10, 5 or less. In some cases, the RNA filter reads may be from about 0 to 10000, 0 to 1000, 0 to 500, 0 to 100, 0 to 50, or 0 to 5.

In some cases, the RNA filter reference length may be at least about 0, 5, 10, 15, 30, 50, 100, 200, 300, 400, 500, 600, 700, 800, 900, 1000, 10000, 20000, 50000 or more. The RNA filter reads may be at most about 50000, 20000, 10000, 1000, 900, 800, 700, 600, 500, 400, 300, 200, 100, 50, 30, 15, 10, 5 or less. The RNA filter reads may be from about 0 to 50000, 0 to 20000, 0 to 10000, 0 to 1000, 0 to 500, 0 to 100, 0 to 50, or 0 to 5.

In some cases, the DNA filter coverage percentage coverage may be at least about 0%, 5%, 10%, 15%, 20%, 25%, 30%, 35%, 40%, 45%, 50%, 55%, 60%, 65%, 70%, 75%, 80%, 85%, 90%, 95%, 99% or more. The DNA filter coverage percentage coverage may be at most about 99%, 95%, 90%, 85%, 80%, 75%, 70%, 65%, 60%, 55%, 50%, 45%, 40%, 35%, 30%, 25%, 20%, 15%, 10%, 5%, or less. The DNA filter coverage percentage coverage may be from about 0% to 100%, 0% to 95%, 0% to 90%, 0% to 85%, 0% to 80%, 0% to 75%, 0% to 70%, 0% to 65%, 0% to 60%, 0% to 55%, 0% to 50%, 0% to 45%, 0% to 40%, 0% to 35%, 0% to 30%, 0% to 25%, 0% to 20%, 0% to 15%, 0% to 10%, or 0% to 5%.

In some cases, the DNA filter average nucleotide identity may be at least about 0%, 5%, 10%, 15%, 20%, 25%, 30%, 35%, 40%, 45%, 50%, 55%, 60%, 65%, 70%, 75%, 80%, 85%, 90%, 95%, 99% or more. The DNA filter average nucleotide identity may be at most about 99%, 95%, 90%, 85%, 80%, 75%, 70%, 65%, 60%, 55%, 50%, 45%, 40%, 35%, 30%, 25%, 20%, 15%, 10%, 5%, or less. The DNA filter average nucleotide identity may be from about 0% to 100%, 0% to 95%, 0% to 90%, 0% to 85%, 0% to 80%, 0% to 75%, 0% to 70%, 0% to 65%, 0% to 60%, 0% to 55%, 0% to 50%, 0% to 45%, 0% to 40%, 0% to 35%, 0% to 30%, 0% to 25%, 0% to 20%, 0% to 15%, 0% to 10%, or 0% to 5%.

In some cases, the DNA filter reads may be at least about 0, 5, 10, 15, 30, 50, 100, 200, 300, 400, 500, 600, 700, 800, 900, 1000, 10000 or more. The DNA filter reads may be at most about 10000, 1000, 900, 800, 700, 600, 500, 400, 300, 200, 100, 50, 30, 15, 10, 5 or less. The DNA filter reads may be from about 0 to 10000, 0 to 1000, 0 to 500, 0 to 100, 0 to 50, or 0 to 5.

In some cases, the DNA filter reference length may be at least about 0, 5, 10, 15, 30, 50, 100, 200, 300, 400, 500, 600, 700, 800, 900, 1000, 10000, 20000, 50000 or more. The DNA filter reads may be at most about 50000, 20000, 10000, 1000, 900, 800, 700, 600, 500, 400, 300, 200, 100, 50, 30, 15, 10, 5 or less. The DNA filter reads may be from about 0 to 50000, 0 to 20000, 0 to 10000, 0 to 1000, 0 to 500, 0 to 100, 0 to 50, or 0 to 5.

In some cases, the filters may be adjusted using a graphical user interface. The filter may be, for example, organism characteristics. Organism characteristics may be, for example, validation status, number of publications, membership in groups, phylogenetic linear, taxonomy, kmer count, or a combination thereof. In some cases, the user may filter using a word and/or text search. In some cases, a filter may be based on artificial intelligence (AI). In some cases, the AI may learn from previous data. In some cases, the AI may report an organism that it classifies as most relevant. In some cases, a filter may be based on a machine learning algorithm. The machine learning algorithm may comprise a deep neural network. The machine learning algorithm may comprise a convolutional neural network.

In some cases, the diagnostic test profile may have at least about 1, 2, 3, 4, 5, 6, 7, 8, 9, 10, 15, 20, 25, 30, 35, 40, 45, 50, 100, 500, 1000 or more filters. In some cases, the diagnostic test profile may have at most about 1000, 500, 100, 50, 45, 40, 35, 30, 25, 20, 15, 10, 9, 8, 7, 6, 5, 4, 3, 2 or less filters. In some cases, the diagnostic test profile may have 1 to 1000, 1 to 100, 1 to 50, 1 to 10, or 1 to 5 filters.

In some cases, the user may adjust the filter at any point in time during data processing. In some cases, the filters are pre-selected by a user and/or computer. In some cases, the filters may be used for more than one diagnostic profile. In some cases, the diagnostic test profile may have the same filters as a different test profile. In some cases, the diagnostic test profile may have different filters than a different test profile.

In some cases, the user may fine-tune criteria for the filters. The criteria may be from the diagnostic test. The criteria may be based on intermediate organism classification results. The criteria may be results from RNA and/or DNA sequences. The criteria may be, for example, the percentage coverage, average nucleotide identity, sequence reads, reference length, or as described elsewhere herein, etc. In some case, the filters may apply a range of values for the criteria. The user may set a range for the criteria. A computer may set the range for the criteria. The range may be any value.

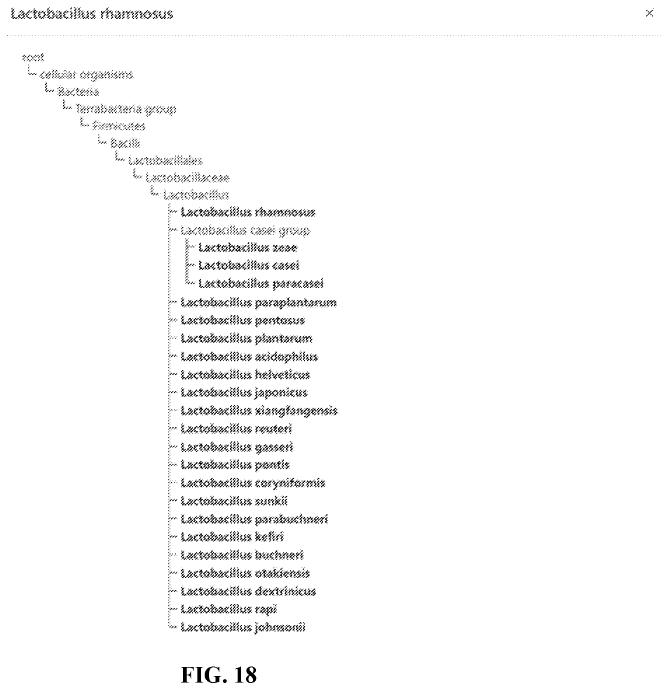

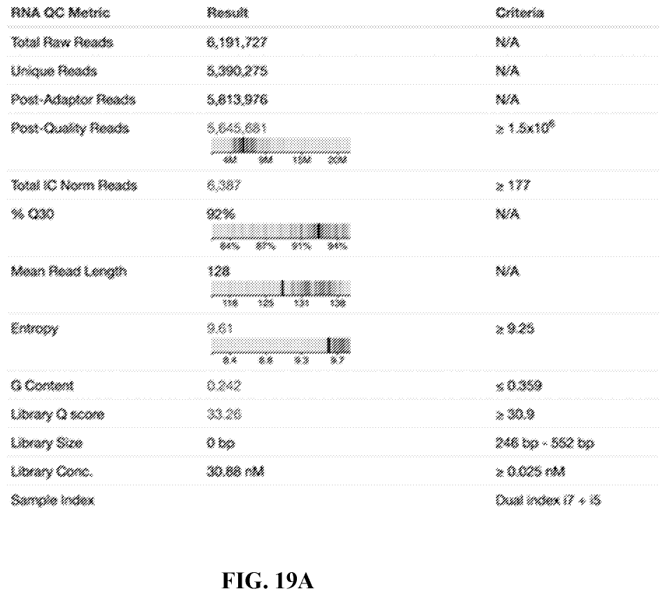

In some cases, the application in accordance with the present disclosure may display to a user one or more results of organism classification. In some cases, the organisms may be unclassified. The organisms may be classified as groups of phylogenetically related organisms. FIG. 18 shows example visualization of classifying organisms. The visualization of the classified organism shows the different members of the phylogenetic tree. The phylogenetic tree shows the possibilities of classes the organism may be from. The class at the top is the one that the software prescribes as the most likely depending on a set of criteria as described elsewhere herein.

In some cases, the members of the classified organisms may be sorted. The member may be sorted depending on criteria, for example, percentage of coverage RNA, percentage of coverage DNA, average nucleotide identity for RNA, average nucleotide identity for DNA, read counts for RNA, or read counts for DNA, or number of relevant publications, etc. In some cases, the sorting may depend on at least about 1, 2, 3, 4, 5, 6, 7, 8, 9, 10, or more criteria. In some cases, the sorting may depend on at most about 10, 9, 8, 7, 6, 5, 4, 3, 2, or less criteria. In some cases, the sorting may depend on 1 to 10, 1 to 8, 1 to 6, 1 to 4, or 1 to 3 criteria.

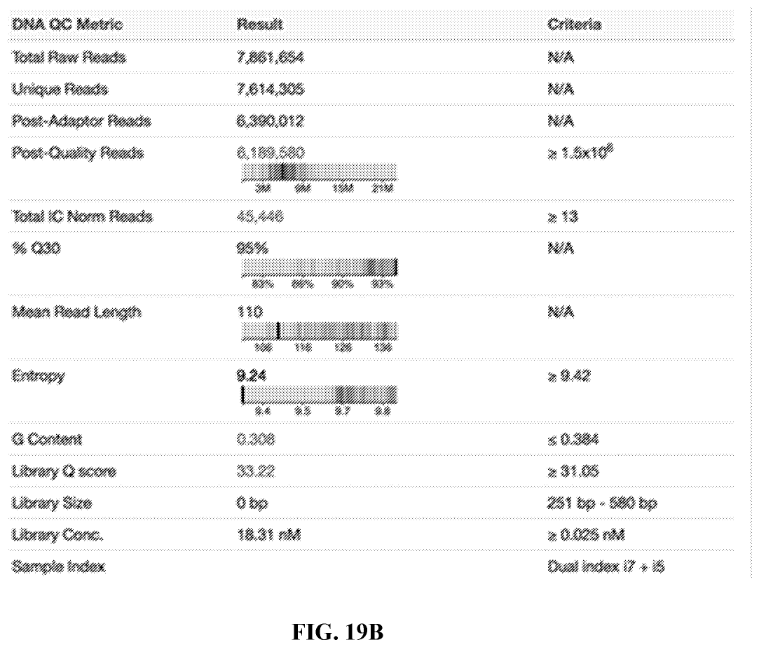

In some cases, the application in accordance with the present disclosure may display to a user quality control metrics as shown in FIG. 19. The metrics may be, for example, total raw reads, unique reads, post-adaptor reads, post-quality reads, total IC norm reads, percentage of bases with a quality score of 30 or higher (% Q30), mean read length, entropy, G Content, library Q score, library size, library concentration, sample index, mean read length, etc. The metrics may be as described elsewhere herein. The metrics may be for RNA metrics and/or DNA metrics. In some cases, the metrics may be displayed. In some cases, the metrics may display a value or number. In some cases, the metrics may be displayed in chart, for example, a horizontal bar chart, vertical bar chart, pie chart, Venn diagram, or any combination thereof. In some cases, the display may display at least about 1, 2, 3, 4, 5, 6, 7, 8, 9, 10, 11, 12, 13, 14, 15, 16, 17, 18, 19, 20, 21, 22, 23, 24, 25, 50, 100, 500, or more metrics. In some cases, the display may display at most about 500, 100, 50, 25, 24, 23, 22, 21, 20, 19, 18, 17, 16, 15, 14, 13, 12, 11, 10, 9, 8, 7, 6, 5, 4, 3, 2, or less metrics. In some cases, the display may display 1 to 500, 1 to 100, 1 to 50, 1 to 25, 1 to 10, or 1 to 5 metrics.

In some cases, mean read length may be after adaptor and quality trimming the reads in the Fastq. In some cases, the reads in the Fastq may be less than in the original demultiplexed Fastq. In some cases, the mean of the shortened reads may give an indication of the extent of trimming.

In some cases, sample index(es) may be the nucleotides (ntd) added to the sequencing libraries that may enable multiplexed sequencing (many sample libraries on one flowcell). In some cases, the number of nucleotides added may be at least about 1, 2, 3, 4, 5, 6, 7, 8, 9, 10, 11, 12, 13, 14, 15 or more. In some cases, the number of nucleotides added may be at most about 15, 14, 13, 12, 11, 10, 9, 8, 7, 6, 5, 4, 3, 2 or less. In some cases, the number of nucleotides added may be from about 1 to 15, 1 to 10, 1 to 5, 3 to 15, 3 to 12, 3 to 10, 3 to 5, 6 to 15, 6 to 12, or 6 to 10. In some cases, the index reads may provide the mechanism to de-multiplex the reads into separate Fastq files.

### AMR Gene Visualization.

The visualization of matching of empirical sequencing data to the references for a condition (e.g., presence of anti-microbial resistance gene in a test sample) may be at the level of protein amino acids. In some cases, the matching of empirical sequencing data to the references for the condition (e.g., AMR gene) may be at the level of nucleotide sequences. In some cases, the matching of empirical sequencing data to the references for a condition (e.g., presence of AMR gene may be at the level of protein amino acids and level of nucleotide sequences. In some cases, the weighting of the matching may be outputted and visualized. The output may be shown as a bit score result. In some cases, the output may be a percent identity. In some cases, the output may comprise a bit score and a percent identity (PID).

In some cases, the AMR genes may be reported out with the detected organisms. In some cases, the AMR genes may be reported without the detected organisms. In some cases, for each reported AMR gene, a variety of characteristics may be displayed. In some cases, the variety of characteristics shown may be at least about 1, 2, 3, 4, 5, 6, 7, 8, 9, 10, 20, 30, 40, 50, 60, 70, 80, 90, 100, 200, 300, 400, 500, 1000, 1500, 2000, 5000, 10000 or more. In some cases, the variety of characteristics shown may be at most about 10000, 5000, 2000, 1500, 1000, 500, 400, 300, 200, 100, 90, 80, 70, 60, 50, 40, 30, 20, 10, 9, 8, 7, 6, 5, 4, 3, 2, or less. In some cases, the variety of characteristics may be from about 1 to 10000, 1 to 1000, 1 to 100, or 1 to 10. The characteristic may be, the name of the gene that confers resistance, the relevant antibiotics, the associated organism(s) where the gene may be found, and a flag to indicate whether the organism can be detected in the sample.

In some cases, a filter may be applied to the AMR gene visualization. The filter may refine or expand the set of AMR genes. The user may apply a filter to avoid false negative results. In some cases, the AMR gene visualization may have at least about 1, 2, 3, 4, 5, 6, 7, 8, 9, 10, 15, 20, 25, 30, 35, 40, 45, 50, 100, 500, 1000 or more filters. In some cases, the AMR gene visualization may have at most about 1000, 500, 100, 50, 45, 40, 35, 30, 25, 20, 15, 10, 9, 8, 7, 6, 5, 4, 3, 2 or less filters. In some cases, the AMR gene visualization may have 1 to 1000, 1 to 100, 1 to 50, 1 to 10, or 1 to 5 filters. In some cases, the user may adjust the filter at any point in time during data processing. In some cases, the filters are pre-selected by a user and/or computer. In some cases, the amount of filters applied may be shown.

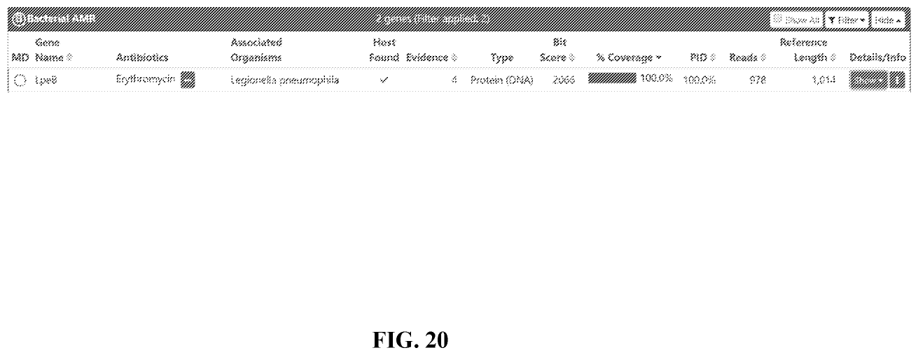

FIG. 20 shows an example visualization of the AMR gene visualization results.

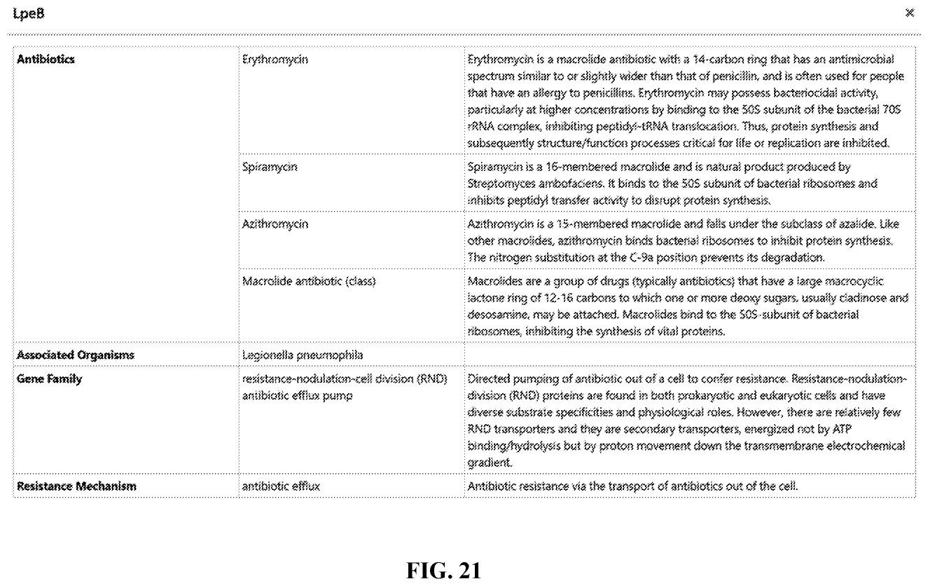

The visualization shows the gene name, antibiotics, associated organism, host found, evidence/publications (as described elsewhere herein), type, bit score, percent coverage, PID, reads, reference length, details, information, and MD. The AMR gene visualization also can be filtered and shows how many filters are currently be applied. The AMR gene visualization has a variety of different clickable buttons that may provide a user with more information. FIG. 21 shows an example visualization of information that the AMR gene visualization provides. The information visualization shows different categories (e.g. antibiotics, associated organisms, gene family, and resistance mechanism). The information visualization provides more information on the subset of categories. The subset of categories are names of antibiotics, or names of associated organisms, etc. The information visualization provides further description to the subcategories. For example, for the category antibiotics the sub category erythromycin is displayed, further a description of erythromycin is provided. In some cases, the description may be inputted by a user or using a natural language processing system. In some cases, the categories and subcategories are inputted by a user or using a computer system.

An AMR gene visualization may link to a details visualization. The details visualization may show 1, 2, 3, 4, 5, 6, 7, 8, 9, 10, 20, 30, 40, 50, 100, 500, 1000, or more details. The details visualization may show at most about 1000, 500, 100, 50, 40, 30, 20, 10, 9, 8, 7, 6, 5, 4, 3, 2, or less details. The details visualization may show about 1 to 1000, 1 to 100, 1 to 10, 1 to 5 details. In some cases, the details may be, for example, coverage plots, bit score cutoff, percent coverage, PID, median depth, reads, reference length, functional annotations, fold coverage vs amino acid position, or fold coverage vs nucleotide, or any combination thereof, etc. In some cases, functional annotations may be, for example protein domains.

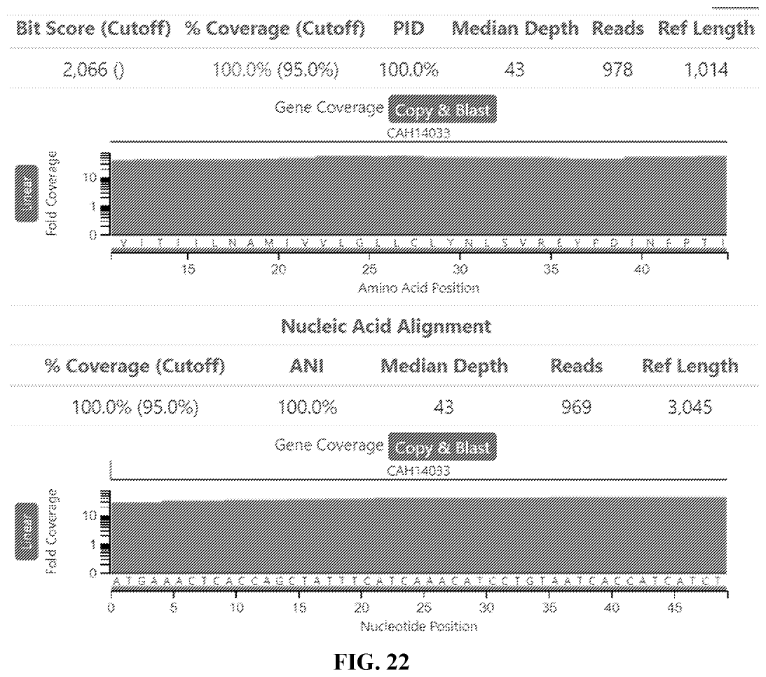

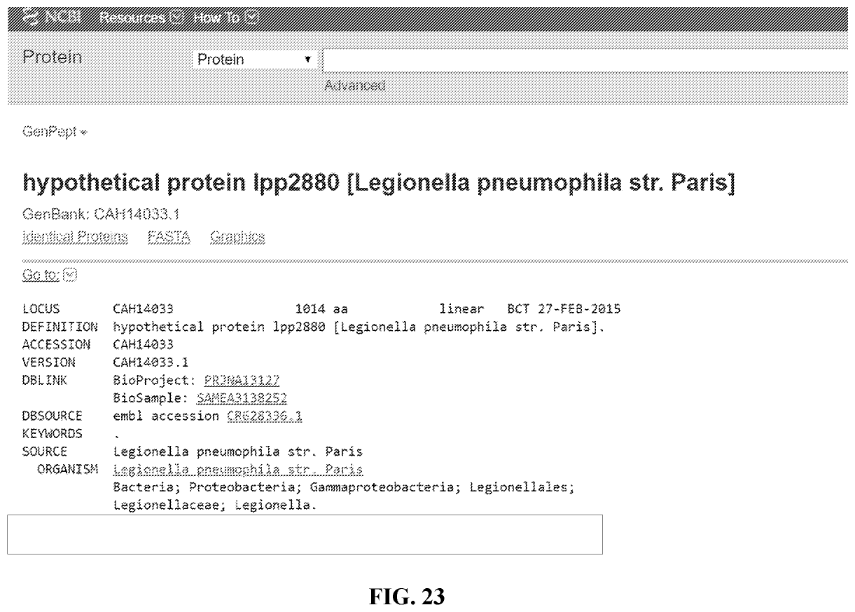

In some cases, the details visualization may display at least about 1, 2, 3, 4, 5, 6, 7, 8, 9, 10, 20, 30, 40, 50, 100, 500, 1000 or more coverage plots. In some cases, the details visualization may display at most about 1000, 500, 100, 50, 40, 30, 20, 10, 9, 8, 7, 6, 5, 4, 3, 2, or less coverage plots. In some cases, the details visualization may display from about 1 to 1000, 1 to 100, or 1 to 10 coverage plots. The coverage plot may show the protein amino acid sequence of the reference and another against the nucleotide sequence of the reference. FIG. 22 shows an example visualization of coverage plots. The x-axis has been zoomed in to view the amino acid sequence and the nucleotide sequence of the reference gene. The coverage plot visualization shows the gene name (e.g. CAH14033) and provides a hyperlink to the user that may open the corresponding record at the NCBI webpage as shown in FIG. 23. Clicking the button (e.g. Copy & Blast) may place the consensus amino sequences into the clipboard in order to conduct a BLAST search. The coverage plot visualization shows the bit score cutoff, percent coverage, PID. median depth, reads, reference length, amino acid position vs fold coverage, and nucleotide position vs fold coverage.

### Visualization of Consensus Sequencing.

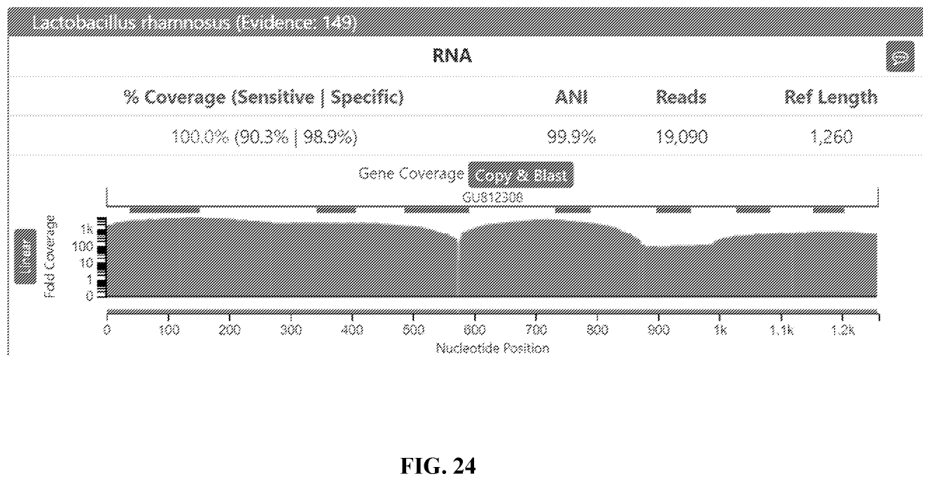

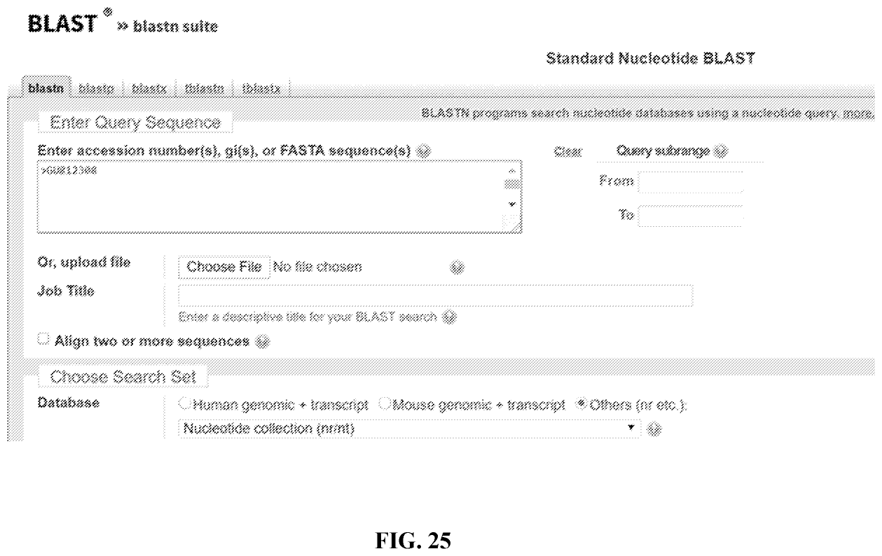

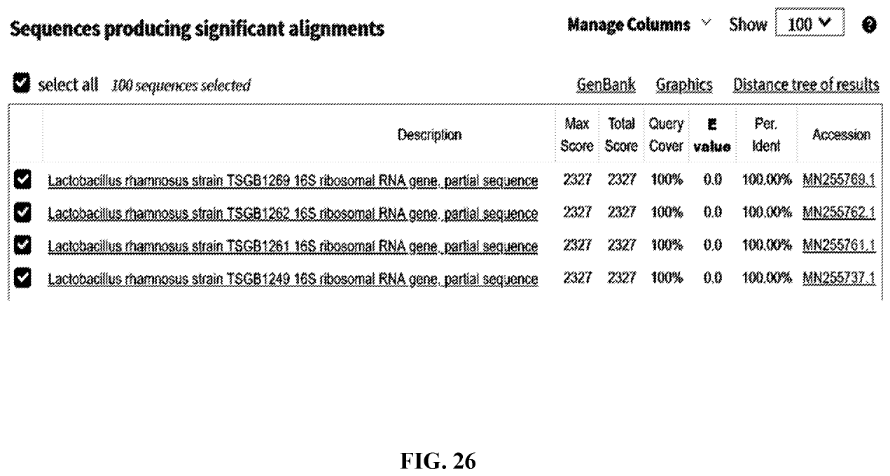

An application may be a web-based application. A web-based application may display a detailed view of detected organisms in the test sample. An example visualization is shown in FIG. 24. The detailed visualization shows the percentage of coverage (e.g., 100%), the sensitive percentage (e.g. 90.3%), the specific percentage (e.g., 98.9%), the average nucleotide identity result (e.g. 99.9%), and the reads (e.g. 19090), the reference length (e.g., 1260), organism name (e.g. Lactobacillus rhamnosus), and evidence/publication count (e.g., 149). The detailed visualization also shows the fold coverage in comparison to the nucleotide position in the form a graph. The detailed visualization also provides a button that places the consensus sequence into the clipboard of the operating system. The button then opens the NCBI BLAST site in a new browser tab. By pasting the consensus sequence(s) into the BLAST Query, a user can run searches against the entire NCBI database collections. FIG. 25 shows an example visualization of the BLAST query. The sequence provided to the BLAST query is from the diagnostic test. FIG. 26 shows an example visualization of example BLAST results. The BLAST result visualization shows the user all sequences and allows the user the option to select all or a subset of sequences. The BLAST result visualization shows a max score, total score, query cover, E value, percent, and accession.

A application in accordance with the present disclosure may display a consensus sequence. In some cases, the web-based application may link and display a NCBI BLAST web page. In some cases, the application of the present disclosure may display a coverage plot. The coverage plot may display coverage of k-mers from empirical sequencing reads. The sequencing reads may be aligned to a reference sequence. In some cases, a consensus sequence (or sequences) may be from assembling sequencing reads. In some cases, a consensus sequence (or sequences) may be compared to the reference sequencing. In some cases, comparing the consensus sequence (or sequences) to a reference sequencing may be the basis for the average nucleotide identity result. In some cases, the detailed visualization may display a button. In some cases, the button may send a user to an external website. In some cases, the button may have a website open within the web-based application. In some cases, the button may have a name (e.g. Copy & Blast). In some cases, the sequence provided to the BLAST query may be from the diagnostic test. In some cases, the button may send the query sequence to blastn, blastp, blastx, tblastn, and/or tblastx.

A BLAST result visualization may show one or more results. In some cases, the results may be description, max score, total score, query cover, E value, percent, accession, distance tree of results, graphics, GenBank. In some cases, the BLAST result visualization may show at least about 1, 2, 3, 4, 5, 6, 7, 8, 9, 10, 20, 30, 40, 50, 60, 70, 80, 90, 100, 500, 1000 or more results. In some cases, the BLAST result visualization may show at most about 1000, 500, 100, 90, 80, 70, 60, 50, 40, 30, 20, 10, 9, 8, 7, 6, 5, 4, 3, 2 or less results. In some cases, the BLAST result visualization may show from about 1 to 1000, 1 to 100, 1 to 20, or 1 to 5 results. The user can at any time decrease or increase the number of results on the BLAST result visualization. The user can also decrease or increase the number of sequences shown on the BLAST result visualization. The user can also download the set of sequences or a subset of sequences. The BLAST results may be saved.

### Analytics.

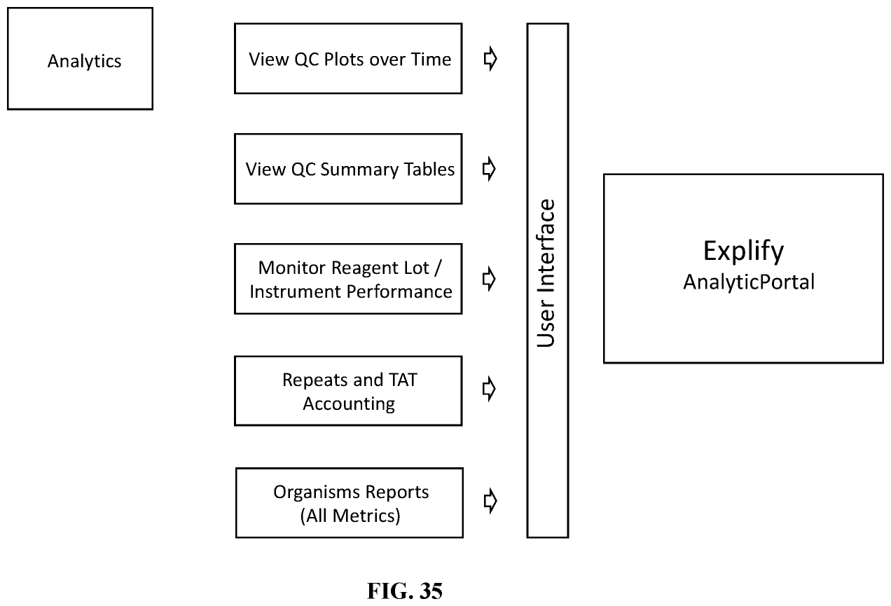

A system or method of the present disclosure may comprise an analytics module. An analytics module may be operatively linked to one or more other modules of a system, including a classification module, interpretation module, detection module, quality control module, laboratory support module, and/or commercial support module. An analytics module may comprise a user interface, which interface may be common to one or more other modules. An analytics module, like an interpretation module, may comprise visualizations and other representations of data. In particular, an analytics module may comprise visualizations and other representations of quality control information. For example, an analytics module may comprise mechanisms for viewing quality control metrics over time and with reference to particular sample types and/or classification processes. Quality control metrics may be represented as, e.g., plots and/or in tabular formats. An analytics module may also facilitate monitoring of reagents and instrument performance, repeat runs, turn-around times, and other performance metrics. An analytics module may also include or provide access to metrics relating to classification information, including organisms reports. An example analytics module is schematically illustrated in FIG. 35.

### Commercial Support

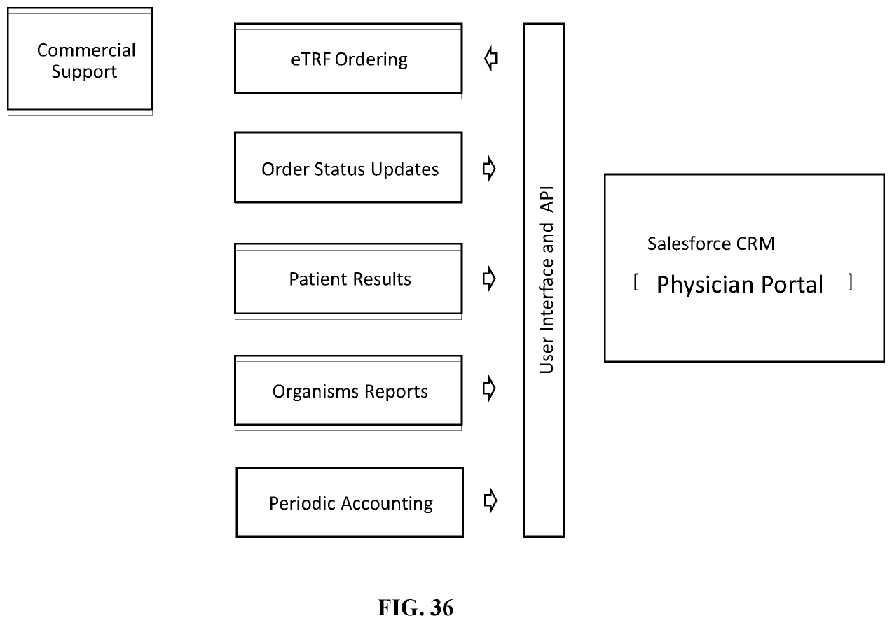

A system or method of the present disclosure may comprise a commercial support module. A commercial support module may be operatively linked to one or more other modules of a system, including a classification module, interpretation module, detection module, quality control module, laboratory support module, and/or analytics module. A commercial support module may comprise a user interface, which interface may be common to one or more other modules. A commercial support module may comprise a mechanism for requesting analysis of a particular sample or type of sample (e.g., electronic test request form ordering), as well as a mechanism for requesting and receiving order status updates. A commercial support module may also comprise various reports including reports regarding particular patients or samples and reports relating to particular organisms or classes of organisms. For example, a commercial support module may comprise a mechanism for viewing the frequency of occurrence of a particular pathogen within a given setting, such as a hospital setting. In an example, a user may be able to view incidences of positive and negative identifications of particular bacteria including Staphylococcus bacteria in different sample types, at different times, and at different locations within a facility, such as a hospital. In this manner, a system of the present disclosure may facilitate tracking of entities including pathogens throughout a facility and patient population. A commercial support module may also facilitate periodic accounting of, e.g., system performance and/or facility performance. An example commercial support module is schematically illustrated in FIG. 36.

### Computer Systems

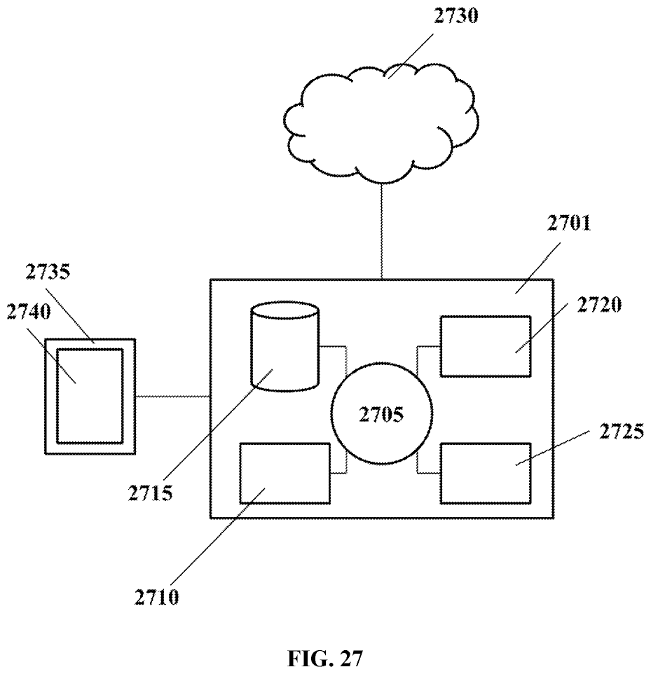

The present disclosure provides computer systems that are programmed to implement methods of the disclosure. FIG. 27 shows a computer system 2701 that is programmed or otherwise configured to calculate k-mers for a sequence, construct consensus sequences from assembling sequencing reads, compare consensus sequences to a reference sequence, display a detailed view of detected organisms, etc. The computer system 2701 can regulate various aspects of parameters of the present disclosure, such as, for example, parameters to calculate k-mers for a sequence, parameters to construct sequences from assembling sequencing reads, parameters of comparing consensus sequences to a reference sequence, etc. The computer system 2701 can be an electronic device of a user or a computer system that is remotely located with respect to the electronic device. The electronic device can be a mobile electronic device.

The computer system 2701 includes a central processing unit (CPU, also “processor” and “computer processor” herein) 2705, which can be a single core or multi core processor, or a plurality of processors for parallel processing. The computer system 2701 also includes memory or memory location 2710 (e.g., random-access memory, read-only memory, flash memory), electronic storage unit 2715 (e.g., hard disk), communication interface 2720 (e.g., network adapter) for communicating with one or more other systems, and peripheral devices 2725, such as cache, other memory, data storage and/or electronic display adapters. The memory 2710, storage unit 2715, interface 2720 and peripheral devices 2725 are in communication with the CPU 2705 through a communication bus (solid lines), such as a motherboard. The storage unit 2715 can be a data storage unit (or data repository) for storing data. The computer system 2701 can be operatively coupled to a computer network (“network”) 2730 with the aid of the communication interface 2720. The network 2730 can be the Internet, an internet and/or extranet, or an intranet and/or extranet that is in communication with the Internet. The network 2730 in some cases is a telecommunication and/or data network. The network 2730 can include one or more computer servers, which can enable distributed computing, such as cloud computing. The network 2730, in some cases with the aid of the computer system 2701, can implement a peer-to-peer network, which may enable devices coupled to the computer system 2701 to behave as a client or a server.

The CPU 2705 can execute a sequence of machine-readable instructions, which can be embodied in a program or software. The instructions may be stored in a memory location, such as the memory 2710. The instructions can be directed to the CPU 2705, which can subsequently program or otherwise configure the CPU 2705 to implement methods of the present disclosure. Examples of operations performed by the CPU 2705 can include fetch, decode, execute, and writeback.

The CPU 2705 can be part of a circuit, such as an integrated circuit. One or more other components of the system 2701 can be included in the circuit. In some cases, the circuit is an application specific integrated circuit (ASIC).

The storage unit 2715 can store files, such as drivers, libraries and saved programs. The storage unit 2715 can store user data, e.g., user preferences and user programs. The computer system 2701 in some cases can include one or more additional data storage units that are external to the computer system 2701, such as located on a remote server that is in communication with the computer system 2701 through an intranet or the Internet.

The computer system 2701 can communicate with one or more remote computer systems through the network 2730. For instance, the computer system 2701 can communicate with a remote computer system of a user. Examples of remote computer systems include personal computers (e.g., portable PC), slate or tablet PC's (e.g., Apple®; iPad, Samsung R Galaxy Tab), telephones, Smart phones (e.g., Apple® iPhone, Android-enabled device, Blackberry®), or personal digital assistants. The user can access the computer system 2701 via the network 2730.

Methods as described herein can be implemented by way of machine (e.g., computer processor) executable code stored on an electronic storage location of the computer system 2701, such as, for example, on the memory 2710 or electronic storage unit 2715. The machine executable or machine readable code can be provided in the form of software. During use, the code can be executed by the processor 2705. In some cases, the code can be retrieved from the storage unit 2715 and stored on the memory 2710 for ready access by the processor 2705. In some situations, the electronic storage unit 2715 can be precluded, and machine-executable instructions are stored on memory 2710.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2701 can include or be in communication with an electronic display 2735 that comprises a user interface (UI) 2740 for providing, for example, a detailed view of detected organisms as described elsewhere herein. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 2705. The algorithm can, for example, calculate k-mers for a sequence, construct consensus sequences from assembling sequencing reads, or compare consensus sequences to a reference sequence, etc.

In certain aspects, the methods provided herein may be computer-implemented methods, where at least one or more steps of the method are carried out by a computer program. In some cases, the methods provided herein are implemented in a computer program stored on computer-readable media, such as the hard drive of a standard computer. For example, a computer program for determining at least one consensus sequence from replicate sequence reads can include one or more of the following: code for providing or receiving the sequence reads, code for identifying regions of sequence overlap between the sequence reads, code for aligning the sequence reads to generate a layout, contig, or scaffold, code for consensus sequence determination, code for converting or displaying the assembly on a computer monitor, code for applying various algorithms described herein, and a computer-readable storage medium comprising the codes.

In some cases, a system (e.g., a data processing system) that may determine at least one assembly from a set of replicate sequences includes a processor, a computer-readable medium operatively coupled to the processor for storing memory, where the memory has instructions for execution by the processor, the instructions including one or more of the following: instructions for receiving input of sequence reads, instructions for overlap detection between the sequence reads, instructions that align the sequence reads to generate a layout, contig, or scaffold, instructions that apply a consensus sequence algorithm to generate at least one consensus sequence (e.g., a “best” consensus sequence, and optionally one or more additional consensus sequences), instructions that compute/store information related to various steps of the method, and instructions that record the results of the method.

In certain cases, various steps of the method may utilize information and/or programs and may generate results that are stored on computer-readable media (e.g., hard drive, auxiliary memory, external memory, server, database, portable memory device (CD-R, DVD, ZIP disk, flash memory cards, etc.), and the like. For example, information used for and results generated by the methods that can be stored on computer-readable media include but are not limited to input sequence read information, set of pair-wise overlaps, newly generated consensus sequences, quality information, technology information, and homologous or reference sequence information.

In some cases, an article of manufacture may provide determining at least one assembly and/or consensus sequence from sequence reads that includes a machine-readable medium containing one or more programs which when executed implement the operations as described herein.

### EXAMPLES

The following examples are given for the purpose of illustrating various embodiments of the invention and are not meant to limit the present invention in any fashion. The present examples, along with the methods described herein are presently representative of preferred embodiments, are merely examples, and are not intended as limitations on the scope of the invention. Changes therein and other uses which are encompassed within the spirit of the invention as defined by the scope of the claims will occur to those skilled in the art.

### Example 1: Example Workflow

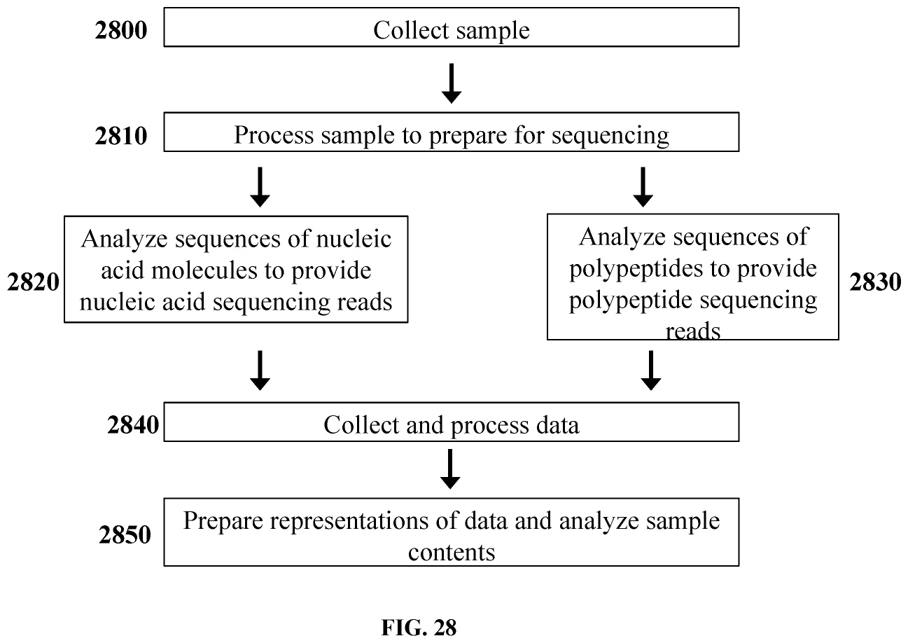

FIG. 28 illustrates an example workflow for the methods provided herein. In item 2800, samples are collected (e.g., as described herein). Samples may be collected from biological sources including human subjects, environmental sources, industrial sources, or other sources. Samples may include fluids and/or solids. Samples may be processed to prepare the samples for subsequent sequencing (2810). Samples may optionally be divided into two or more portions for subsequent analysis. Samples that may be analyzed for nucleic acids included therein may be process and/or analyzed separately from samples that may be analyzed for polypeptides included therein. Sequences of nucleic acid molecules and/or polypeptides of the sample may be analyzed using nucleic acid and/or polypeptide sequencing techniques (2820 and 2830). Data prepared from this analysis, including sequencing reads, may be collected and optionally combined. Data may be stored locally and/or in a web- or cloud-based storage system. Data may be compared against sequences in one or more reference databases (e.g., as described herein) (2840). Data may be processed and interpreted using a software program, such as a web-based software program. A user may prepare and/or interpret various representations of the data. The data may be analyzed to interpret the nucleic acid molecules and/or polypeptides included in the sample, thereby identifying microorganisms, viruses, genes, or other contents of the sample (2850). A variety of representations of the data may be prepared (e.g., as described herein). Such representations and reports may be used to inform a variety of interventions including medical interventions and physical interventions (e.g., as described herein). For example, a report may be used to inform a treatment regimen for a patient.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A computer system comprising:
one or more processors;
a memory; and
one or more programs, wherein the one or more programs are stored in the memory and configured to be executed by the one or more processors, the one or more programs for identifying a presence or an absence of one or more conditions in a first sample from a sample source, the one or more programs comprising:
(A) a classification module that includes instructions for:
(i) obtaining, in electronic form, a set of sample sequence reads for a plurality of polynucleotides from the first sample,
(ii) performing, for each respective sequence read in the set of sample sequence reads a comparison between at least a portion of the respective sample sequence read and each reference sequence in a first set of reference sequences, thereby performing a first plurality of sequence comparisons,
(iii) calculating, from the first plurality of sequence comparisons, a respective probability that the respective sample sequence read corresponds to a particular reference sequence in the first set of reference sequences thereby computing a first plurality of probabilities, and
(iv) identifying a presence or an absence of each of the one or more conditions in the sample based at least in part on the first plurality of probabilities.

2. The computer system of claim 1, wherein the performing (A)(ii) comprises forming a respective plurality of k-mers that represent the respective sample sequence read and comparing each k-mer to a corresponding plurality of weighted k-mers representing a reference sequence, in polynucleotide form, in the first set of reference sequences.

3. The computer system of claim 2, wherein a k-mer weight of a respective weighted k-mer in the corresponding plurality of weighted k-mers for a reference sequence relates to a count of a particular k-mer within a particular reference sequence, a count of the particular k-mer among a group of sequences comprising the reference sequence, and a count of the particular k-mer among all reference sequences in the set of reference sequences.

4. The computer system of claim 2, wherein;
a respective weighted k-mer (Kᵢ) in the corresponding plurality of weighted k-mers for a reference sequence (refᵢ) in the first set of reference sequences has a higher weight KWrefᵢ when it is a less prevalent k-mer across the first set of reference sequences, in polynucleotide form, and
a respective weighted k-mer (Kᵢ) in the corresponding plurality of weighted k-mers for a reference sequence (refᵢ) in the first set of reference sequences has a lower weight KWrefᵢ when it is a more prevalent k-mer across the reference sequence, in polynucleotide form.

5. The computer system of claim 2, wherein the first set of reference sequences are protein sequences and the one or more programs further comprise instructions for translating the first set of reference sequence to polynucleotide form.

6. The computer system of claim 2, wherein KWrefᵢ is calculated as:
$$K W r e {f}_{i} = \frac{{C}_{ref} ( {K}_{i} ) / {C}_{d b} ( {K}_{i} )}{{C}_{d b} ( {K}_{i} ) / Total kmer count}$$
wherein,
C_{ref}(Kᵢ) is a count of a number of occurrences of the respective weighted k-mer (Kᵢ) in the respective reference sequence (refᵢ),
C_{db}(Kᵢ) is a count of a number of occurrences of the respective k-mer (Kᵢ) in the first set of reference sequences, and
Total kmer count is a number of k-mers of length k-nucleotides in the first set of reference sequences.

7.-17. (canceled)

18. The computer system of claim 1, wherein a condition in the one or more conditions is presence of nucleic acids or proteins in the first sample from a particular taxa.

19. (canceled)

20. The computer system of claim 1, wherein the sample source is a test subject and wherein a condition in the one or more conditions is presence of an expression profile, a particular gene, a particular antimicrobial resistance gene, a particular antiviral resistance gene, a particular antivirulent resistance gene, a particular antiparasitic resistant gene, or a particular antiprotozoal resistance gene in the first sample.

21. (canceled)

22. The computer system of claim 1, wherein a condition in the one or more conditions is a taxa and the taxa comprises a first bacterial strain identified as present in the sample source and a second bacterial strain identified as absent from the sample source.

23. The computer system of claim 1, wherein the first set of reference sequences consist of between 100 and 1×10⁶ groups of sequences, wherein each respective group of sequences is associated with a different bacterial or viral contaminant and each condition in the one or more conditions corresponds to a different group in the between 100 and 1×10⁶ groups of sequences.

24. (canceled)

25. (canceled)

26. The computer system of claim 1, wherein the first set of reference sequences comprises sequences from a plurality of taxa, and a reference sequence in the first set of reference sequences is associated with a reference k-mer weight indicative of a likelihood that a reference k-mer within the reference polynucleotide sequence originates from a taxon.

27. The computer system of claim 1, wherein the first set of reference sequences includes reference sequences for 10, 50, 100, 1000, 10000, 100000, 1000000, or more conditions.

28. (canceled)

29. (canceled)

30. The computer system of claim 27, wherein each corresponding set of one or more genetic variants includes an epigenetic modification.

31.-33. (canceled)

34. The computer system of claim 1, wherein the one or more programs further comprises instructions for determining an absolute or relative abundance of a composition, associated with a condition in the one or more conditions, in the first sample.

35.-42. (canceled)

43. The computer system of claim 1, wherein the first set of reference sequences comprises reference sequences of one or more of bacteria, archaea, chromalveolata, viruses, fungi, plants, fish, amphibians, reptiles, birds, mammals, and humans.

44.-46. (canceled)

47. The computer system of claim 1, wherein the first set of reference sequences comprises a plurality of marker gene sequences for taxonomic classification of bacterial sequences.

48.-70. (canceled)

71. The computer system of claim 1, wherein
the first set of reference sequences are nucleotide sequences;
the second set of reference sequences are protein sequence;
each sequence comparison performed by the A(ii) sequence comparison is a nucleotide sequence to nucleotide sequence comparison, and
each sequence comparison performed by the A(iii) sequence comparison is an amino acid sequence to amino acid sequence comparison in which the respective sample sequence read or sample contig has been translated to an amino acid sequence.

72. The computer system of claim 71, wherein the A(ii) sequence comparison is performed for each of six different reference frames of the respective sample sequence read or respective sample contig.

73.-75. (canceled)

76. The computer system of claim 1 wherein a condition in the one or more conditions is an identification of a first species present in the first sample, and the one or more programs further comprises instructions for showing a percentage of a genome of the first species identified by the (A)(ii) in the set of sample sequence reads.

77. The computer system of claim 1 wherein each respective condition in the one or more conditions is an identification of a corresponding species in a plurality of species identified as present in the first sample, and the one or more programs further comprises instructions for showing a respective percentage of a corresponding genome identified by the (A)(ii) in the set of sample sequence reads for each species in the plurality of species.

78.-103. (canceled)
